(19) United States
(12) Patent Application Publication
(10) Pub. No.: US 2026/0196321 A1
(43) Pub. Date: Jul. 9, 2026

(54) ACTION CONTROL SYSTEM AND INFORMATION PROCESSING SYSTEM

(71) Applicant: SOFTBANK GROUP CORP., Tokyo (JP)

(72) Inventor: Masayoshi SON, Tokyo (JP)

(73) Assignee: SOFTBANK GROUP CORP., Tokyo (JP)

(21) Appl. No.: 19/461,765

(22) Filed: Jan. 28, 2026

Related U.S. Application Data
(63) Continuation of application No. PCT/JP2024/026644, filed on Jul. 25, 2024.

(30) Foreign Application Priority Data
Aug. 1, 2023 (JP) 2023-125788
Aug. 1, 2023 (JP) 2023-125790
Aug. 2, 2023 (JP) 2023-126181
Aug. 2, 2023 (JP) 2023-126501
Aug. 3, 2023 (JP) 2023-127361
Aug. 3, 2023 (JP) 2023-127388
Aug. 3, 2023 (JP) 2023-127391
Aug. 3, 2023 (JP) 2023-127392
Aug. 3, 2023 (JP) 2023-127395
Aug. 4, 2023 (JP) 2023-128180
Aug. 4, 2023 (JP) 2023-128185
Aug. 4, 2023 (JP) 2023-128186
Aug. 7, 2023 (JP) 2023-128896
Aug. 8, 2023 (JP) 2023-129640
Aug. 9, 2023 (JP) 2023-130526
Aug. 9, 2023 (JP) 2023-130527
Aug. 10, 2023 (JP) 2023-131170
Aug. 10, 2023 (JP) 2023-131172
Aug. 10, 2023 (JP) 2023-131231
Aug. 10, 2023 (JP) 2023-131576
Aug. 14, 2023 (JP) 2023-131822
Aug. 14, 2023 (JP) 2023-131844
Aug. 14, 2023 (JP) 2023-131845
Aug. 15, 2023 (JP) 2023-132319
Aug. 17, 2023 (JP) 2023-133098
Aug. 17, 2023 (JP) 2023-133117
Aug. 17, 2023 (JP) 2023-133118
Aug. 17, 2023 (JP) 2023-133136
Aug. 31, 2023 (JP) 2023-141857

(51) Int. Cl. G16H 20/00; G06F 3/01; G06T 13/40; G16H 40/67

(52) U.S. Cl. CPC G16H 20/00; G06F 3/015; G06T 13/40; G16H 40/67

(57) ABSTRACT

An avatar is caused to perform an action appropriate for an action of a user. In an action control system, in a case in which an action of the avatar includes dreaming and the action determination unit determines dreaming, as the action of the avatar, the action determination unit creates an original event obtained by combining multiple pieces of event data among pieces of data in history data.

### CROSS-REFERENCE TO RELATED APPLICATIONS

This application is a continuation application of International Application No. PCT/JP2024/026644, filed Jul. 25, 2024, which claims priority from Japanese Patent Application No. 2023-125788, filed Aug. 1, 2023, Japanese Patent Application No. 2023-125790, filed Aug. 1, 2023, Japanese Patent Application No. 2023-126181, filed Aug. 2, 2023, Japanese Patent Application No. 2023-126501, filed Aug. 2, 2023, Japanese Patent Application No. 2023-127361, filed Aug. 3, 2023, Japanese Patent Application No. 2023-127388, filed Aug. 3, 2023, Japanese Patent Application No. 2023-127391, filed Aug. 3, 2023, Japanese Patent Application No. 2023-127392, filed Aug. 3, 2023, Japanese Patent Application No. 2023-127395, filed Aug. 3, 2023, Japanese Patent Application No. 2023-128180, filed Aug. 4, 2023, Japanese Patent Application No. 2023-128185, filed Aug. 4, 2023, Japanese Patent Application No. 2023-128186, filed Aug. 4, 2023, Japanese Patent Application No. 2023-128896, filed Aug. 7, 2023, Japanese Patent Application No. 2023-129640, filed Aug. 8, 2023, Japanese Patent Application No. 2023-130526, filed Aug. 9, 2023, Japanese Patent Application No. 2023-130527, filed Aug. 9, 2023, Japanese Patent Application No. 2023-131170, filed Aug. 10, 2023, Japanese Patent Application No. 2023-131172, filed Aug. 10, 2023, Japanese Patent Application No. 2023-131231, filed Aug. 10, 2023, Japanese Patent Application No. 2023-131576, filed Aug. 10, 2023, Japanese Patent Application No. 2023-131822, filed Aug. 14, 2023, Japanese Patent Application No. 2023-131844, filed Aug. 14, 2023, Japanese Patent Application No. 2023-131845, filed Aug. 14, 2023, Japanese Patent Application No. 2023-132319, filed Aug. 15, 2023, Japanese Patent Application No. 2023-133098, filed Aug. 17, 2023, Japanese Patent Application No. 2023-133117, filed Aug. 17, 2023, Japanese Patent Application No. 2023-133118, filed Aug. 17, 2023, Japanese Patent Application No. 2023-133136, filed Aug. 17, 2023, Japanese Patent Application No. 2023-141857, filed Aug. 31, 2023, the disclosures of each are incorporated herein by reference in their entirety.

### TECHNICAL FIELD

The present disclosure relates to an action control system and an information processing system.

### BACKGROUND ART

Patent Literature 1 discloses a technique for determining an appropriate action of a robot for a state of a user. In the related art of Patent Literature 1, in a case in which a robot has recognized a user's reaction in a case in which the robot executed a specific action and an action of the robot in response to the recognized user's reaction has not been determined, the action of the robot is updated by receiving information regarding the action suitable for the user's recognized state from a server.

Patent Literature 2 discloses a persona chatbot control method executed by at least one processor, the method including: receiving a user utterance; adding the user utterance to a prompt including an instructional sentence associated with a description of a character of a chatbot; encoding the prompt; and inputting the encoded prompt into a language model to generate a chatbot utterance that is responsive to the user utterance.

### PRIOR ART DOCUMENT

### Patent Literature

Patent Literature 1: Japanese Patent Publication No. 6053847
Patent Literature 2: Japanese Patent Application Laid-Open (JP-A) No. 2022-180282

### SUMMARY OF INVENTION

### Technical Problem

However, in the related art, there is room for improvement in causing the robot to execute an appropriate action for the user's action.

Further, at the time of earthquake alert, only information such as seismic intensity, magnitude, and depth of seismic source is obtained in a studio of a television station. Therefore, the announcer only announces to the viewers preset phrases such as, “Please be careful of tsunami just in case. Do not approach cliffs or the like. Repeat.”, so it is difficult for the viewers to take measures against earthquakes.

### Solution to Problem

According to a first aspect of the disclosure, an action control system is provided. The action control system includes:
a state recognition unit that recognizes a user state including an action of a user and a state of electronic equipment;
an emotion determination unit that determines an emotion of the user or an emotion of an avatar representing an agent for interacting with the user;
an action determination unit that uses at least one of the user state, the state of the electronic equipment, the emotion of the user, or the emotion of the avatar, together with an action determination model at a predetermined timing to determine any of multiple types of avatar actions including not acting, as an action of the avatar;
a memory control unit that causes event data including an emotion value determined by the emotion determination unit and data including the action of the user to be stored in history data; and
an action control unit that displays the avatar in an image display area of the electronic equipment,
in which the avatar actions include dreaming, and
in a case in which the action determination unit determines dreaming as an action of the avatar, the action determination unit creates an original event obtained by combining multiple pieces of event data among pieces of data in the history data.

According to a second aspect of the disclosure, the action determination model is a data generation model capable of generating data according to input data, and the action determination unit inputs data indicating at least one of the user state, the state of the electronic equipment, the emotion of the user, or the emotion of the avatar, together with data for asking about the avatar action to the data generation model, and determines an action of the avatar based on an output of the data generation model.

According to a third aspect of the disclosure, in a case in which the action determination unit determines dreaming as an action of the avatar, the action determination unit causes the action control unit to control the avatar so as to generate the original event.

According to a fourth aspect of the disclosure, the electronic equipment is a headset-type terminal.

According to a fifth aspect of the disclosure, the electronic equipment is an eyeglass-type terminal.

According to a sixth aspect of the disclosure, an action control system is provided. That action control system includes a state recognition unit that recognizes a user state including an action of a user and a state of electronic equipment; an emotion determination unit that determines an emotion of the user or an emotion of an avatar representing an agent for interacting with the user; an action determination unit that uses at least one of the user state, the state of the electronic equipment, the emotion of the user, or the emotion of the avatar, together with an action determination model at a predetermined timing to determine any of multiple types of avatar actions including not acting, as an action of the avatar; a memory control unit that causes event data including an emotion value determined by the emotion determination unit and data including the action of the user to be stored in history data; and an action control unit that displays the avatar in an image display area of the electronic equipment, in which the avatar actions include proposing an activity, and in a case in which the action determination unit determines to propose an activity as an action of the avatar, the action determination unit determines an action of the user to propose based on the event data.

According to a seventh aspect of the disclosure, an action control system is provided. The action control system includes a state recognition unit that recognizes a user state including an action of a user and a state of electronic equipment; an emotion determination unit that determines an emotion of the user or an emotion of an avatar representing an agent for interacting with the user; an action determination unit that uses at least one of the user state, the state of the electronic equipment, the emotion of the user, or the emotion of the avatar, together with an action determination model at a predetermined timing to determine any of multiple types of avatar actions including not acting, as an action of the avatar; a memory control unit that causes event data including an emotion value determined by the emotion determination unit and data including the action of the user to be stored in history data; and an action control unit that displays the avatar in an image display area of the electronic equipment, in which the avatar actions include comforting the user, and in a case in which the action determination unit determines to comfort the user as an action of the avatar, the action determination unit determines an utterance content corresponding to the user state and the emotion of the user.

Here, the electronic equipment may be a robot, and the robot includes a device that performs a physical operation, a device that outputs a video or a sound without performing a physical operation, and an agent that operates on software.

According to an eighth aspect of the disclosure, an action control system is provided. An action control system includes a state recognition unit that recognizes a user state including an action of a user and a state of electronic equipment; an emotion determination unit that determines an emotion of the user or an emotion of an avatar representing an agent for interacting with the user; an action determination unit that uses at least one of the user state, the state of the electronic equipment, the emotion of the user, or the emotion of the avatar, together with an action determination model at a predetermined timing to determine any of multiple types of avatar actions including not acting, as an action of the avatar; and an action control unit that displays the avatar in an image display area of the electronic equipment, in which the avatar actions include presenting a question to the user, and in a case in which the action determination unit determines to present a question to the user as an action of the avatar, the action determination unit creates a question to be presented to the user.

According to a ninth aspect of the disclosure, an action control system is provided. The action control system includes a state recognition unit that recognizes a user state including an action of a user and a state of electronic equipment; an emotion determination unit that determines an emotion of the user or an emotion of an avatar representing an agent for interacting with the user; an action determination unit that uses at least one of the user state, the state of the electronic equipment, the emotion of the user, or the emotion of the avatar, together with an action determination model at a predetermined timing to determine any of multiple types of avatar actions including not acting, as an action of the avatar; and an action control unit that displays the avatar in an image display area of the electronic equipment, in which the avatar actions include teaching music, and in a case in which the action determination unit determines to teach music as an action of the avatar, the action determination unit evaluates a sound generated by the user.

According to a tenth aspect of the disclosure, an action control system is provided. The action control system includes a state recognition unit that recognizes a user state including an action of a user and a state of electronic equipment; an emotion determination unit that determines an emotion of the user or an emotion of an avatar representing an agent for interacting with the user; an action determination unit that uses at least one of the user state, the state of the electronic equipment, the emotion of the user, or the emotion of the avatar, together with an action determination model at a predetermined timing to determine any of multiple types of avatar actions including not acting, as an action of the avatar; and an action control unit that displays the avatar in an image display area of the electronic equipment, in which the avatar actions include presenting a question to the user, and in a case in which the action determination unit determines to present a question to the user as an action of the avatar, the action determination unit presents a question suitable for the user based on a content of a text used by the user and a target deviation value of the user.

According to an eleventh aspect of the disclosure, an action control system is provided. The action determination model of the action control system is a data generation model capable of generating data according to input data, and the action determination unit inputs data indicating at least one of the user state, the state of the electronic equipment, the emotion of the user, or the emotion of the avatar, together with data for asking about the avatar action to the data generation model, and determines an action of the avatar based on an output of the data generation model.

According to a twelfth aspect of the disclosure, an action control system is provided. When the action determination unit of the action control system determines that, as the emotion of the user, the user is in a state in which the user appears to be bored or the user is scolded to study by a guardian of the user, the action determination unit presents a question suitable for the user.

According to a thirteenth aspect of the disclosure, an action control system is provided. In a case in which the user is able to correctly answer the presented question, the action determination unit of the action control system presents a question requiring an answer of a higher difficulty.

According to a fourteenth aspect of the disclosure, an action control system is provided. The electronic equipment of the action control system is a headset-type terminal.

According to a fifteenth aspect of the disclosure, an action control system is provided. The electronic equipment of the action control system is an eyeglass-type terminal.

Here, a robot includes a device that performs a physical operation, a device that outputs a video or a sound without performing a physical operation, and an agent that operates on software.

According to a sixteenth aspect of the disclosure, an action control system is provided. The action control system includes a state recognition unit that recognizes a user state including an action of a user and a state of electronic equipment; an emotion determination unit that determines an emotion of the user or an emotion of an avatar representing an agent for interacting with the user; an action determination unit that uses at least one of the user state, the state of the electronic equipment, the emotion of the user, or the emotion of the avatar, together with an action determination model at a predetermined timing to determine any of multiple types of avatar actions including not acting, as an action of the avatar; and an action control unit that displays the avatar in an image display area of the electronic equipment, in which the avatar actions include giving advice on a specific competition to the user participating in the specific competition, the action determination unit includes an image acquisition unit that can capture a competition space in which the specific competition that the user is participating in is being held, and a feature identifying unit that identifies features of a plurality of players competing in the specific competition in the competition space captured by the image acquisition unit, and in a case in which the action determination unit determines, as an action of the avatar, to give advice on the specific competition to the user participating in the specific competition, advice is given to the user based on an identified result of the feature identifying unit.

According to a seventeenth aspect of the disclosure, an action control system is provided. The action control system includes a state recognition unit that recognizes a user state including an action of a user and a state of electronic equipment; an emotion determination unit that determines an emotion of the user or an emotion of an avatar representing an agent for interacting with the user; an action determination unit that uses at least one of the user state, the state of the electronic equipment, the emotion of the user, or the emotion of the avatar, together with an action determination model at a predetermined timing to determine any of multiple types of avatar actions including not acting, as an action of the avatar; a memory control unit that causes event data including an emotion value determined by the emotion determination unit and data including an action of the user to be stored in history data; and an action control unit that displays the avatar in an image display area of the electronic equipment, in which the avatar actions include setting a first action content for correcting an action of the user, and in a case in which the action determination unit spontaneously or periodically detects an action of the user and determines, as an action of the avatar, to correct the action of the user, based on the detected action of the user and specific information stored in advance, the action determination unit causes the action control unit to display the avatar in an image display area so as to implement the first action content.

According to an eighteenth aspect of the disclosure, an action control system is provided. The action control system includes a state recognition unit that recognizes a user state including an action of a user and a state of electronic equipment; an emotion determination unit that determines an emotion of the user or an emotion of an avatar representing an agent for interacting with the user; an action determination unit that uses at least one of the user state, the state of the electronic equipment, the emotion of the user, or the emotion of the avatar, together with an action determination model at a predetermined timing to determine any of multiple types of avatar actions including not acting, as an action of the avatar; and an action control unit that displays the avatar in an image display area of the electronic equipment. The avatar actions include giving advice on a social networking service to the user, and in a case in which the action determination unit determines, as an action of the avatar, to give advice on a social networking service to the user, advice on a social networking service is given to the user.

Here, a robot includes a device that performs a physical operation, a device that outputs a video or a sound without performing a physical operation, and an agent that operates on software.

According to a nineteenth aspect of the disclosure, an action control system is provided. The action control system includes a state recognition unit that recognizes a user state including an action of a user and a state of electronic equipment; an emotion determination unit that determines an emotion of the user or an emotion of an avatar representing an agent for interacting with the user; an action determination unit that uses at least one of the user state, the state of the electronic equipment, the emotion of the user, or the emotion of the avatar, together with an action determination model at a predetermined timing to determine any of multiple types of avatar actions including not acting, as an action of the avatar; a memory control unit that causes event data including an emotion value determined by the emotion determination unit and data including the action of the user to be stored in history data; and an action control unit that displays the avatar in an image display area of the electronic equipment, in which the avatar actions include giving advice on caregiving to the user, and in a case in which the action determination unit determines, as an action of the avatar, to give advice on caregiving to the user, the action determination unit collects information about caregiving of the user and gives advice on caregiving of the user based on the collected information.

Here, a robot includes a device that performs a physical operation, a device that outputs a video or a sound without performing a physical operation, and an agent that operates on software.

According to a twentieth aspect of the disclosure, an action control system is provided. The action control system includes a state recognition unit that recognizes a user state including an action of a user and a state of electronic equipment; an emotion determination unit that determines an emotion of the user or an emotion of an avatar representing an agent for interacting with the user; an action determination unit that uses at least one of the user state, the state of the electronic equipment, the emotion of the user, or the emotion of the avatar, together with an action determination model at a predetermined timing to determine any of multiple types of avatar actions including not acting, as an action of the avatar; and an action control unit that displays the avatar in an image display area of the electronic equipment, in which the avatar actions include giving advice on a risk approaching the user, and in a case in which the action determination unit determines to give advice on a risk approaching the user as an action of the avatar, advice on the risk approaching the user is given.

According to a twenty-first aspect of the disclosure, an action control system is provided. The action control system includes a state recognition unit that recognizes a user state including an action of a user and a state of electronic equipment; an emotion determination unit that determines an emotion of the user or an emotion of an avatar representing an agent for interacting with the user; an action determination unit that uses at least one of the user state, the state of the electronic equipment, the emotion of the user, or the emotion of the avatar, together with an action determination model at a predetermined timing to determine any of multiple types of avatar actions including not acting, as an action of the avatar; a memory control unit that causes event data including an emotion value determined by the emotion determination unit and data including the action of the user to be stored in history data; and an action control unit that displays the avatar in an image display area of the electronic equipment, in which the avatar actions include giving advice on health to the user, and in a case in which the action determination unit determines, as an action of the avatar, to give advice on health to the user, advice on health is given to the user.

According to a twenty-second aspect of the disclosure, an action control system is provided. The action control system includes a state recognition unit that recognizes a user state including an action of a user and a state of electronic equipment; an emotion determination unit that determines an emotion of the user or an emotion of an avatar representing an agent for interacting with the user; an action determination unit that uses at least one of the user state, the state of the electronic equipment, the emotion of the user, or the emotion of the avatar, together with an action determination model at a predetermined timing to determine any of multiple types of avatar actions including not acting, as an action of the avatar; a memory control unit that causes event data including an emotion value determined by the emotion determination unit and data including the action of the user to be stored in history data; and an action control unit that displays the avatar in an image display area of the electronic equipment, in which the avatar actions include autonomously converting speech of the user into a question, and in a case in which the action determination unit determines, as an action of the avatar, to convert speech of the user into a question and answer the question, the action determination unit converts the speech of the user into a question and answers the question by using a sentence generation model based on the event data.

According to a twenty-third aspect of the disclosure, an action control system is provided. The action control system includes a state recognition unit that recognizes a user state including an action of a user and a state of electronic equipment; an emotion determination unit that determines an emotion of the user or an emotion of an avatar representing an agent for interacting with the user; an action determination unit that uses at least one of the user state, the state of the electronic equipment, the emotion of the user, or the emotion of the avatar, together with an action determination model at a predetermined timing to determine any of multiple types of avatar actions including not acting, as an action of the avatar; and an action control unit that displays the avatar in an image display area of the electronic equipment. The avatar actions include increasing a vocabulary and uttering about the increased vocabulary, and in a case in which the action determination unit determines to increase a vocabulary and utter the increased vocabulary as an action of the avatar, the action determination unit increases the vocabulary and utters the increased vocabulary.

Here, a robot includes a device that performs a physical operation, a device that outputs a video or a sound without performing a physical operation, and an agent that operates on software.

A twenty-fourth aspect of the disclosure is an action control system including a state recognition unit that recognizes a user state including an action of a user and a state of electronic equipment; an emotion determination unit that determines an emotion of the user or an emotion of an avatar representing an agent for interacting with the user; an action determination unit that uses at least one of the user state, the state of the electronic equipment, the emotion of the user, or the emotion of the avatar, together with an action determination model at a predetermined timing to determine any of multiple types of avatar actions including not acting, as an action of the avatar; a memory control unit that causes event data including an emotion value determined by the emotion determination unit and data including an action of the user to be stored in history data; and an action control unit that displays the avatar in an image display area of the electronic equipment, in which the avatar actions include learning an utterance method and changing a setting for the utterance method, and in a case in which the action determination unit determines, as an action of the avatar, to learn the utterance method, utterances of a speaker in a preset information source are collected, and in a case in which the action determination unit determines, as an action of the avatar, to change the settings for the utterance method, a voice emitted is changed according to an attribute of the user.

In a twenty-fifth aspect of the disclosure, the action determination model is a data generation model capable of generating data according to input data, and the action determination unit inputs data indicating at least one of the user state, the state of the electronic equipment, the emotion of the user, or the emotion of the avatar, together with data for asking about the avatar action to the data generation model, and determines an action of the avatar based on an output of the data generation model.

In a twenty-sixth aspect of the disclosure, the electronic equipment is a headset, and the action determination unit determines an action of the avatar controlled by the action control unit as a part of an image displayed in the image display area of the headset, and determines any of multiple types of avatar actions including not acting, as an action of the avatar.

In a twenty-seventh aspect of the disclosure, the action determination model is a sentence generation model having an interaction function, and the action determination unit inputs a text indicating at least one of the user state, the state of the avatar displayed in the image display area, the emotion of the user, or the emotion of the avatar displayed in the image display area and a text for asking about an action of the avatar to the sentence generation model, and determines an action of the avatar based on an output of the sentence generation model.

In a twenty-eighth aspect of the disclosure, in a case in which it is determined to change the setting for the utterance method as an action of the avatar, the action control unit operates the avatar with a look corresponding to the voice emitted after the change.

A twenty-ninth aspect of the disclosure is an action control system including a state recognition unit that recognizes a user state including an action of a user and a state of electronic equipment; an emotion determination unit that determines an emotion of the user or an emotion of an avatar representing an agent for interacting with the user; an action determination unit that uses at least one of the user state, the state of the electronic equipment, the emotion of the user, or the emotion of the avatar, together with an action determination model at a predetermined timing to determine any of multiple types of avatar actions including not acting, as an action of the avatar; a memory control unit that causes event data including an emotion value determined by the emotion determination unit and data including an action of the user to be stored in history data; and an action control unit that displays the avatar in an image display area of the electronic equipment, in which the avatar actions include learning an utterance method and changing a setting for the utterance method, and in a case in which the action determination unit determines, as an action of the avatar, to learn the utterance method, utterances of a speaker in a preset information source are collected, and in a case in which the action determination unit determines, as an action of the avatar, to change the settings for the utterance method, a voice emitted is changed according to an attribute of the user.

In a thirtieth aspect of the disclosure, the action determination model is a data generation model capable of generating data according to input data, and the action determination unit inputs data indicating at least one of the user state, the state of the electronic equipment, the emotion of the user, or the emotion of the avatar, together with data for asking about the avatar action to the data generation model, and determines an action of the avatar based on an output of the data generation model.

In a thirty-first aspect of the disclosure, the electronic equipment is a headset, and the action determination unit determines an action of the avatar controlled by the action control unit as a part of an image displayed in the image display area of the headset, and determines any of multiple types of avatar actions including not acting, as an action of the avatar.

In a thirty-second aspect of the disclosure, the action determination model is a sentence generation model having an interaction function, and the action determination unit inputs a text indicating at least one of the user state, the state of the avatar displayed in the image display area, the emotion of the user, or the emotion of the avatar displayed in the image display area and a text for asking about an action of the avatar to the sentence generation model, and determines an action of the avatar based on an output of the sentence generation model.

In a thirty-third aspect of the disclosure, in a case in which it is determined to change the setting for the utterance method as an action of the avatar, the action control unit operates the avatar with a look corresponding to the voice emitted after the change.

According to a thirty-fourth aspect of the disclosure, a state recognition unit that recognizes a user state including an action of a user and a state of electronic equipment; an emotion determination unit that determines an emotion of the user or an emotion of an avatar representing an agent for interacting with the user; an action determination unit that uses at least one of the user state, the state of the electronic equipment, the emotion of the user, or the emotion of the avatar, together with an action determination model at a predetermined timing to determine any of multiple types of avatar actions including not acting, as an action of the avatar; and an action control unit that displays the avatar in an image display area of the electronic equipment are included, the avatar actions include considering a mental age of the user, and in a case in which the action determination unit determines to consider the mental age of the user as an action of the avatar, the action determination unit estimates the mental age of the user and determines the avatar action in accordance with the estimated mental age of the user.

According to a thirty-fifth aspect of the disclosure, an action control system is provided. The action control system includes a state recognition unit that recognizes a user state including an action of a user and a state of electronic equipment; an emotion determination unit that determines an emotion of the user or an emotion of an avatar representing an agent for interacting with the user; an action determination unit that uses at least one of the user state, the state of the electronic equipment, the emotion of the user, or the emotion of the avatar, together with an action determination model at a predetermined timing to determine any of multiple types of avatar actions including not acting, as an action of the avatar; a memory control unit that causes event data including an emotion value determined by the emotion determination unit and data including the action of the user to be stored in history data; and an action control unit that displays the avatar in an image display area of the electronic equipment, in which the avatar actions include estimating a foreign language level of the user and conversing with the user in a foreign language, and in a case in which the action determination unit determines to estimate a foreign language level of the user as an action of the avatar, the action determination unit estimates the foreign language level of the user, and in a case in which the action determination unit determines to converse with the user in a foreign language, the action determination unit converses with the user in the foreign language.

According to a thirty-sixth aspect of the disclosure, an action control system is provided. The action control system includes a state recognition unit that recognizes a user state including an action of a user and a state of electronic equipment; an emotion determination unit that determines an emotion of the user or an emotion of an avatar representing an agent for interacting with the user; an action determination unit that uses at least one of the user state, the state of the electronic equipment, the emotion of the user, or the emotion of the avatar, together with an action determination model at a predetermined timing to determine any of multiple types of avatar actions including not acting, as an action of the avatar; and an action control unit that displays the avatar in an image display area of the electronic equipment, in which the avatar actions include giving advice on a creative activity of the user to the user, and in a case in which the action determination unit determines, as an action of the avatar, to give advice on a creative activity of the user to the user, the action determination unit collects information regarding the creative activity of the user and gives advice on the creative activity of the user based on the collected information.

Here, a robot is a device that performs a physical operation, a device that outputs a video or a sound without performing a physical operation, and an agent that operates on software.

According to a thirty-seventh aspect of the disclosure, an action control system is provided. An action control system includes a state recognition unit that recognizes a user state including an action of a user and a state of electronic equipment; an emotion determination unit that determines an emotion of the user or an emotion of an avatar representing an agent for interacting with the user; an action determination unit that uses at least one of the user state, the state of the electronic equipment, the emotion of the user, or the emotion of the avatar, together with an action determination model at a predetermined timing to determine any of multiple types of avatar actions including not acting, as an action of the avatar; a memory control unit that causes event data including an emotion value determined by the emotion determination unit and data including an action of the user to be stored in history data; and an action control unit that displays the avatar in an image display area of the electronic equipment, in which the avatar actions include proposing to encourage an action that the user is able to take in the home, the memory control unit stores a type of an action of the user performed in the home in the history data in association with a timing at which the action is performed, and in a case in which the action determination unit spontaneously or periodically determines, as an action of the avatar, to propose to encourage the action that the user is able to take in the home based on the history data, the action determination unit causes the action control unit to display the avatar in the image display area so as to make the proposal to encourage the action at a timing at which the user should perform the action.

According to a thirty-eighth aspect of the disclosure, an action control system is provided. The action control system includes a state recognition unit that recognizes a user state including an action of a user and a state of electronic equipment; an emotion determination unit that determines an emotion of the user or an emotion of an avatar representing an agent for interacting with the user; an action determination unit that uses at least one of the user state, the state of the electronic equipment, the emotion of the user, or the emotion of the avatar, together with an action determination model at a predetermined timing to determine any of multiple types of avatar actions including not acting, as an action of the avatar; and an action control unit that displays the avatar in an image display area of the electronic equipment, in which the avatar actions include making an utterance or a gesture by the electronic equipment to the user, and the action determination unit determines a content of the utterance or the gesture and causes the action control unit to control the avatar so as to provide learning support to the user based on a sensory characteristic of the user.

According to a thirty-ninth aspect of the disclosure, an action control system is provided. The action control system includes:
a state recognition unit that recognizes a user state including an action of a user and a state of electronic equipment;
an emotion determination unit that determines an emotion of the user or an emotion of an avatar representing an agent for interacting with the user;
an action determination unit that uses at least one of the user state, the state of the electronic equipment, the emotion of the user, or the emotion of the avatar, together with an action determination model at a predetermined timing to determine any of multiple types of avatar actions including not acting, as an action of the avatar; and
an action control unit that displays the avatar in an image display area of the electronic equipment,
in which the action determination unit determines an action content of the avatar so as to acquire a lyric and a music score of a melody according to an environment in which the electronic equipment is placed based on the action determination model, play music based on the lyric and melody using a sound synthesis engine, sing along with the music, and/or dance to the music.

According to a fortieth aspect of the disclosure, the action determination model is a data generation model capable of generating data according to input data, and
the action determination unit inputs data indicating at least one of the environment in which the electronic equipment is placed, the user state, the state of the electronic equipment, the emotion of the user, or the emotion of the avatar, together with data for asking about the avatar action to the data generation model, and determines an action of the avatar based on an output of the data generation model.

According to a forty-first aspect of the disclosure, the action control unit controls the avatar so as to play the music, sing along with the music, and/or dance to the music.

According to a forty-second aspect of the disclosure, the electronic equipment is a headset-type terminal.

According to a forty-third aspect of the disclosure, the electronic equipment is an eyeglass-type terminal.

According to a forty-fourth aspect of the disclosure, an action control system is provided. The action control system includes a state recognition unit that recognizes a user state including an action of a user and a state of electronic equipment; an emotion determination unit that determines an emotion of the user or an emotion of an avatar representing an agent for interacting with the user; an action determination unit that determines an action of the avatar based on at least the user state, the state of the electronic equipment, the emotion of the user, or the emotion of the avatar; and an action control unit that displays the avatar in an image display area of the electronic equipment, in which, in a case in which the action determination unit determines, as an action of the avatar, to answer a question of the user, the action determination unit acquires a vector indicating the question of the user, searches a database that stores a combination of a question and an answer for a question having a vector corresponding to the acquired vector, and generates an answer to the question of the user by using an answer to the question that is searched for and a sentence generation model that is capable of generating a sentence in accordance with input data.

According to a forty-fifth aspect of the disclosure, an information processing system is provided. The information processing system includes an input unit that accepts a user input; a processing unit that performs a specific process using a sentence generation model that generates a sentence according to input data; an output unit that controls an action of electronic equipment so as to output a result of the specific process; and an action control unit that displays an avatar in an image display area of the electronic equipment, in which, in a case in which pitch information regarding a ball to be thrown next by a specific pitcher is requested, the processing unit performs, as the specific process, a process of generating a sentence instructing creation of the pitch information accepted by the input unit and inputting the generated sentence to the sentence generation model, and causes the output unit to output the created pitch information to the avatar representing an agent for interacting with the user as a result of the specific process.

According to a forty-sixth aspect of the disclosure, an information processing system is provided. The information processing system includes an input unit that accepts a user input; a processing unit that performs a specific process using a generation model configured to generate a result according to input data; and an output unit that displays an avatar that represents an agent for interacting with a user in an image display area of electronic equipment so as to output a result of the specific process, in which the processing unit uses an output of the generation model when a text instructing presentation of information regarding an earthquake is set as the input data, acquires the information regarding the earthquake as a result of the specific process, and causes the information to be output to the avatar.

According to a forty-seventh aspect of the disclosure, an action control system is provided. The action control system includes:
a state recognition unit that recognizes a user state including an action of a user and a state of electronic equipment;
an emotion determination unit that determines an emotion of the user or an emotion of an avatar representing an agent for interacting with the user;
an action determination unit that uses at least one of the user state, the state of the electronic equipment, the emotion of the user, or the emotion of the avatar, together with an action determination model at a predetermined timing to determine any of multiple types of avatar actions including not acting, as an action of the avatar; and
an action control unit that displays the avatar in an image display area of the electronic equipment,
in which the action determination unit analyzes a social networking service (social media) related to the user by using the action determination model, recognizes a matter that the user is interested in based on a result of the analysis, and determines an action content of the avatar so as to provide the user with information based on the recognized matter.

According to a forty-eighth aspect of the disclosure, the action determination model is a data generation model capable of generating data according to input data, and
the action determination unit inputs data indicating at least one of the environment in which the electronic equipment is placed, the user state, the state of the electronic equipment, the emotion of the user, or the emotion of the avatar, together with data for asking about the avatar action to the data generation model, and determines an action of the avatar based on an output of the data generation model.

According to a forty-ninth aspect of the disclosure, the action control unit controls the avatar so as to provide the user with the information based on the recognized matter.

According to a fiftieth aspect of the disclosure, the electronic equipment is a headset-type terminal.

According to a fifty-first aspect of the disclosure, the electronic equipment is an eyeglass-type terminal.

A fifty-second aspect of the disclosure is an action control system including a state recognition unit that recognizes a user state including an action of a user and a state of electronic equipment; an emotion determination unit that determines an emotion of the user or an emotion of an avatar representing an agent for interacting with the user; an action determination unit that uses at least one of the user state, the state of the electronic equipment, the emotion of the user, or the emotion of the avatar, together with an action determination model at a predetermined timing to determine, as an action of the avatar, any of multiple types of avatar actions including not acting, as an action of the avatar; and an action control unit that displays the avatar in an image display area of the electronic equipment, in which, in a case in which the user is determined as a specific user including an individual living alone in solitude, the action determination unit switches, as an action of the avatar, to a specific mode in which an action of the avatar is determined at a higher communication frequency than a communication frequency in a normal mode in which an action of the avatar is determined for the user different from the specific user.

In a fifty-third aspect of the disclosure, the action determination model is a data generation model capable of generating data according to input data, and the action determination unit inputs data indicating at least one of the user state, the state of the electronic equipment, the emotion of the user, or the emotion of the avatar, together with data for asking about the avatar action to the data generation model, and determines an action of the avatar based on an output of the data generation model.

In a fifty-fourth aspect of the disclosure, the electronic equipment is a headset, and the action determination unit determines an action of the avatar controlled by the action control unit as a part of an image displayed in the image display area of the headset, and determines any of multiple types of avatar actions including not acting, as an action of the avatar.

In a fifty-fifth aspect of the disclosure, the action determination model is a sentence generation model having an interaction function, and the action determination unit inputs a text indicating at least one of the user state, the state of the avatar displayed in the image display area, the emotion of the user, or the emotion of the avatar displayed in the image display area and a text for asking about an action of the avatar to the sentence generation model, and determines an action of the avatar based on an output of the sentence generation model.

A fifty-sixth aspect of the disclosure is an action control system including a state recognition unit that recognizes a user state including an action of a user and a state of electronic equipment; an emotion determination unit that determines an emotion of the user or an emotion of an avatar representing an agent for interacting with the user; an action determination unit that uses at least one of the user state, the state of the electronic equipment, the emotion of the user, or the emotion of the avatar, together with an action determination model at a predetermined timing to determine any of multiple types of avatar actions including not acting, as an action of the avatar; and an action control unit that displays the avatar in an image display area of the electronic equipment, in which, the action determination unit sets, as an interaction mode of the avatar, a customer service interaction mode in which someone can be designated as an interaction partner when the user does not need to talk to a specific person but wants someone to listen to the user's talk, and in the customer service interaction mode, the action determination unit excludes a predetermined keyword related to the specific person in an interaction with the user and outputs an utterance content.

According to a fifty-seventh aspect of the disclosure, the action determination model is a data generation model capable of generating data according to input data, and the action determination unit inputs data indicating at least one of the user state, the state of the electronic equipment, the emotion of the user, or the emotion of the avatar, together with data for asking about the avatar action to the data generation model, and determines an action of the avatar based on an output of the data generation model.

According to a fifty-eighth aspect of the disclosure, the electronic equipment is a headset, and the action determination unit determines an action of the avatar controlled by the action control unit as a part of an image displayed in the image display area of the headset, and determines any of multiple types of avatar actions including not acting, as an action of the avatar.

According to a fifty-ninth aspect of the disclosure, the action determination model is a sentence generation model having an interaction function, and the action determination unit inputs a text indicating at least one of the user state, the state of the avatar displayed in the image display area, the emotion of the user, or the emotion of the avatar displayed in the image display area and a text for asking about an action of the avatar to the sentence generation model, and determines an action of the avatar based on an output of the sentence generation model.

According to a sixtieth aspect of the disclosure, in a case in which it is determined to change a setting of the interaction partner in the customer service interaction mode as an action of the avatar, the action control unit causes the avatar to operate with an utterance and a look corresponding to the changed interaction partner.

According to a sixty-first aspect of the disclosure, an action control system is provided. The action control system includes an action determination unit that determines an action of an avatar representing an agent for interacting with a user; and an action control unit that displays the avatar in an image display area of electronic equipment, in which an image sensor or an odor sensor is set at customs, the action determination unit acquires an image of a person by using the image sensor or an odor detection result by using the odor sensor, and in a case in which a preset abnormal action, abnormal expression, or abnormal odor is detected, the action determination unit determines to notify a customs inspector of the detection as an action of the avatar.

### BRIEF DESCRIPTION OF DRAWINGS

FIG. 1 schematically illustrates an example of a system 5 according to a first embodiment.

FIG. 2 schematically illustrates a functional configuration of a robot 100 according to the first embodiment.

FIG. 3 schematically shows an example of an operation flow of a collecting process by the robot 100 according to the first embodiment.

FIG. 4A schematically shows an example of an operation flow of a response process by the robot 100 according to the first embodiment.

FIG. 4B schematically shows an example of an operation flow of an autonomous process by the robot 100 according to the first embodiment.

FIG. 5 illustrates an emotion map 400 on which multiple emotions are mapped.

FIG. 6 illustrates an emotion map 900 on which multiple emotions are mapped.

FIG. 7(A) is an external view of a stuffed toy 100N according to a second embodiment, and FIG. 7(B) is an internal structural view of the stuffed toy 100N.

FIG. 8 is a rear front view of the stuffed toy 100N according to the second embodiment.

FIG. 9 schematically illustrates a functional configuration of the stuffed toy 100N according to the second embodiment.

FIG. 10 schematically illustrates a functional configuration of an agent system 500 according to a third embodiment.

FIG. 11 illustrates an example of an operation of the agent system.

FIG. 12 illustrates an example of an operation of the agent system.

FIG. 13 schematically illustrates a functional configuration of an agent system 700 according to a fourth embodiment.

FIG. 14 illustrates an example of a usage mode of the agent system using smart glasses.

FIG. 15 schematically illustrates a functional configuration of an agent system 800 according to a fifth embodiment.

FIG. 16 illustrates an example of a headset-type terminal.

FIG. 17 schematically illustrates an example of a hardware configuration of a computer 1200.

FIG. 18A schematically illustrates another functional configuration of a robot 100.

FIG. 18B schematically illustrates a functional configuration of a specific processing unit of the robot 100.

FIG. 19 illustrates an outline of specific process.

FIG. 20 schematically shows an example of an operation flow of the specific process by the robot 100.

FIG. 21 schematically illustrates an example of an operation flow related to an operation in which the robot 100 performs a specific process of supporting the user 10 for announcement of information regarding an earthquake.

### DESCRIPTION OF EMBODIMENTS

Hereinafter, embodiments of the disclosure will be described, and the following embodiments do not limit the invention according to the claims. In addition, not all combinations of features described in the embodiments are essential to the solution of the disclosure.

### First Embodiment

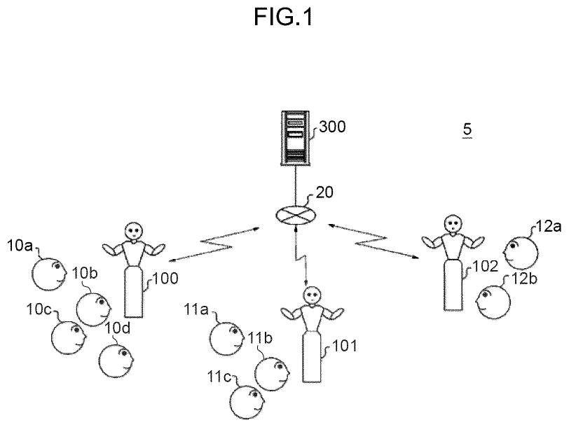

FIG. 1 schematically illustrates an example of a system 5 according to the present embodiment. The system 5 includes a robot 100, a robot 101, a robot 102, and a server 300. A user 10a, a user 10b, a user 10c, and a user 10d are users of the robot 100. A user 11a, a user 11b, and a user 11c are users of the robot 101. A user 12a and a user 12b are users of the robot 102. Note that, in the description of the present embodiment, the user 10a, the user 10b, the user 10c, and the user 10d may be collectively referred to as “user 10”. Furthermore, the user 11a, the user 11b, and the user 11c may be collectively referred to as “user 11”. Furthermore, the user 12a and the user 12b may be collectively referred to as “user 12”. The robot 101 and the robot 102 have substantially the same functions as those of the robot 100. Thus, the system 5 will be described focusing on the functions of the robot 100.

The robot 100 has conversations with the user 10 and provides videos to the user 10. At this time, the robot 100 performs a conversation with the user 10 and provides a video to the user 10, and the like in cooperation with the server 300 and the like that can communicate via a communication network 20. For example, the robot 100 not only learns an appropriate conversation by itself, but also performs learning so that a conversation with the user 10 can be advanced more appropriately in cooperation with the server 300. Further, the robot 100 causes the server 300 to record captured video data and the like of the user 10, requests the server 300 for the video data and the like if necessary, and provides the video data and the like to the user 10.

Furthermore, the robot 100 has an emotion value indicating the type of its own emotion. For example, the robot 100 has emotion values indicating the intensity of each emotion such as “joy”, “anger”, “sorrow”, “pleasure”, “comfort”, “discomfort”, “relief”, “anxiety”, “sadness”, “excitement”, “worry”, “reassurance”, “fulfillment”, “emptiness”, and “neutral”. For example, in a case in which the robot 100 has a conversation with the user 10 with a high emotion value of excitement, the robot emits voice at a fast speed. As described above, the robot 100 can express its own emotion by action.

Furthermore, the robot 100 may be configured to determine an action of the robot 100 corresponding to an emotion of the user 10 by matching a sentence generation model using artificial intelligence (AI) with an emotion engine. Specifically, the robot 100 may be configured to recognize an action of the user 10, determine the emotion of the user 10 for the action of the user, and determine an action of the robot 100 corresponding to the determined emotion.

More specifically, in a case in which the robot 100 has recognized an action of the user 10, the robot 100 automatically generates the action content to be taken by the robot 100 in response to the action of the user 10 by using a preset sentence generation model. The sentence generation model may be interpreted as an algorithm and an arithmetic operation for an automatic interaction process based on characters. Since the sentence generation model is known as disclosed in, for example, Japanese Patent Application Laid-Open (JP-A) No. 2018-081444 and ChatGPT (retrieved from the Internet <URL: https://openai.com/blog/chatgpt>), detailed description thereof will be omitted. Such a sentence generation model is configured by a large-scale language model (LLM).

As described above, in the present embodiment, it is possible to reflect the emotions of the user 10 and the robot 100 and various linguistic information in actions of the robot 100 by combining the large-scale language model and the emotion engine. That is, according to the present embodiment, synergistic effects can be obtained by combining the sentence generation model and the emotion engine.

Further, the robot 100 has the function of recognizing actions of the user 10. The robot 100 recognizes actions of the user 10 by analyzing face images of the user 10 acquired by the camera function and voices of the user 10 acquired by the microphone function. The robot 100 determines an action to be performed by the robot 100 based on a recognized action of the user 10 or the like.

As an example of an action determination model, the robot 100 stores a rule for defining an action to be performed by the robot 100 based on an emotion of the user 10, an emotion of the robot 100, and an action of the user 10, and performs various actions according to the rule.

Specifically, the robot 100 includes, as an example of the action determination model, reaction rules for determining an action of the robot 100 based on an emotion of the user 10, an emotion of the robot 100, and an action of the user 10. According to the reaction rules, for example, in a case in which an action of the user 10 is “laughing”, the action of the robot 100 is set to “laughing”. In addition, according to the reaction rules, in a case in which an action of the user 10 is “getting angry”, the action of the robot 100 is set to “apologizing”. In addition, according to the reaction rules, in a case in which an action of the user 10 is “asking a question”, the action of the robot 100 is set to “answering”. According to the reaction rules, in a case in which an action of the user 10 is “expressing sadness”, the action of the robot 100 is set to “showing encouragement”.

In a case in which the robot 100 recognizes the action of the user 10 as “getting angry” based on the reaction rules, the robot chooses the action of “apologizing” defined in the reaction rules as an action to be performed by the robot 100. For example, in the case of choosing the action of “apologizing”, the robot 100 performs the action of “apologizing” and outputs a voice expressing a word of “apology”.

Furthermore, in a case in which a condition that the emotion of the robot 100 is “neutral” (that is, “joy”=0, “anger”=0, “sadness”=0, and “pleasure”=0) and the state of the user 10 is “being alone is lonely” is satisfied, it is defined that the content of emotion change in the emotion of the robot 100 to “worried” and the action of “showing encouragement” can be performed.

In a case in which the robot 100 recognizes that the current emotion of the robot 100 is “neutral” and the user 10 is alone and feels sad based on the reaction rules, the emotion value of “sorrow” of the robot 100 is increased. Furthermore, the robot 100 selects an action of “showing encouragement” defined in the reaction rule as an action to be performed on the user 10. For example, in a case in which the action of “showing encouragement” is selected, the robot 100 converts the phrase “What's wrong?” expressing concern into a voice expressing concern, and outputs the voice.

Furthermore, the robot 100 transmits, to the server 300, user reaction information indicating that a positive reaction has been obtained from the user 10 due to this action. The user reaction information includes, for example, a user action of “getting angry”, an action of the robot 100 of “apologizing”, a positive reaction of the user 10, and an attribute of the user 10.

The server 300 stores the user reaction information received from the robot 100. Note that the server 300 receives the user reaction information not only from the robot 100 but also from each of the robot 101 and the robot 102 and stores the user reaction information. Then, the server 300 analyzes the user reaction information from the robot 100, the robot 101, and the robot 102, and updates the reaction rules.

The robot 100 inquires the server 300 about the updated reaction rules to receive the updated reaction rules from the server 300. The robot 100 incorporates the updated reaction rules into the reaction rules stored in the robot 100. As a result, the robot 100 can incorporate the reaction rules acquired by the robot 101, the robot 102, and the like into its own reaction rules.

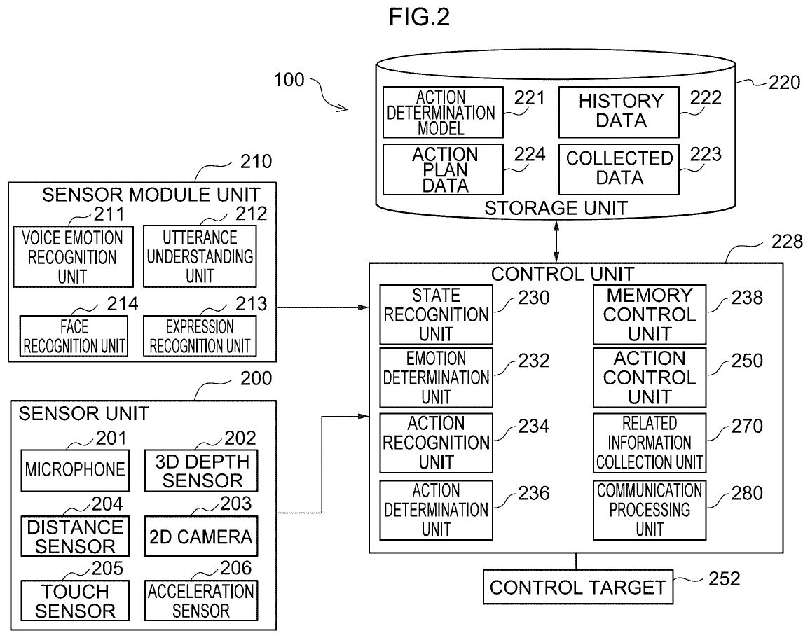

FIG. 2 schematically illustrates a functional configuration of the robot 100. The robot 100 includes a sensor unit 200, a sensor module unit 210, a storage unit 220, a control unit 228, and a control target 252. The control unit 228 includes a state recognition unit 230, an emotion determination unit 232, an action recognition unit 234, an action determination unit 236, a memory control unit 238, an action control unit 250, a related information collection unit 270, and a communication processing unit 280.

The control target 252 includes a display device, a speaker, an LED at the eye part, motors that drive arms, hands, feet, and the like. Postures and gestures of the robot 100 are controlled by controlling motors for arms, hands, and feet. Some of the emotions of the robot 100 can be expressed by controlling these motors. Furthermore, expressions of the robot 100 can be represented by controlling light emission states of the LEDs at the eye part of the robot 100. Note that the postures, gestures, and expressions of the robot 100 are examples of attitudes of the robot 100.

The sensor unit 200 includes a microphone 201, a 3D depth sensor 202, a 2D camera 203, a distance sensor 204, a touch sensor 205, and an acceleration sensor 206. The microphone 201 continuously detects sound and outputs voice data. Note that the microphone 201 may be provided on the head of the robot 100 and may have a function of performing binaural recording. The 3D depth sensor 202 detects outlines of an object by continuously emitting an infrared pattern and analyzing the infrared pattern from an infrared image continuously captured by an infrared camera. The 2D camera 203 is an example of an image sensor. The 2D camera 203 captures an image with visible light and generates image information from visible light. The distance sensor 204 detects a distance to an object by emitting, for example, a laser, an ultrasonic wave, or the like. Note that the sensor unit 200 may further include a clock, a gyro sensor, a sensor for motor feedback, and the like.

Note that, among the components of the robot 100 illustrated in FIG. 2, the components other than the control target 252 and the sensor unit 200 are examples of the components included in the action control system of the robot 100. The control target 252 is a target to be controlled by the action control system of the robot 100.

The storage unit 220 includes an action determination model 221, history data 222, collected data 223, and action plan data 224. The history data 222 includes past emotion values of the user 10, past emotion values of the robot 100, and an action history, and specifically includes multiple pieces of event data including the emotion values of the user 10, the emotion values of the robot 100, and actions of the user 10. The data including the actions of the user 10 includes camera images representing the actions of the user 10. The emotion values and the action history are recorded for each user 10 by being associated with identification information of the user 10, for example. At least a part of the storage unit 220 is implemented by a storage medium such as a memory. A person DB that stores face images of the user 10, attribute information of the user 10, and the like may be included. Note that, among the components of the robot 100 illustrated in FIG. 2, the functions of the components other than the control target 252, the sensor unit 200, and the storage unit 220 can be realized by a CPU operating according to programs. For example, the functions of these components can be implemented as operations of the CPU by basic software (OS) and programs operating on the OS.

The sensor module unit 210 includes a voice emotion recognition unit 211, an utterance understanding unit 212, an expression recognition unit 213, and a face recognition unit 214. Information detected by the sensor unit 200 is input to the sensor module unit 210. The sensor module unit 210 analyzes information detected by the sensor unit 200 and outputs the analysis result to the state recognition unit 230.

The voice emotion recognition unit 211 of the sensor module unit 210 analyzes a voice of the user 10 detected by the microphone 201 to recognize the emotion of the user 10. For example, the voice emotion recognition unit 211 extracts a feature such as a frequency component of the utterance and recognizes the emotion of the user 10 based on the extracted feature. The utterance understanding unit 212 analyzes the voice of the user 10 detected by the microphone 201 and outputs character information indicating the utterance content of the user 10.

The expression recognition unit 213 recognizes the facial expression of the user 10 and the emotion of the user 10 from an image of the user 10 captured by the 2D camera 203. For example, the expression recognition unit 213 recognizes the facial expression and emotion of the user 10 based on the shapes, positional relationships, and the like of the user's eyes and mouth.

The face recognition unit 214 recognizes the face of the user 10. The face recognition unit 214 recognizes the user 10 by matching a face image stored in the person DB (not illustrated) with a face image of the user 10 captured by the 2D camera 203.

The state recognition unit 230 recognizes the state of the user 10 based on the information analyzed by the sensor module unit 210. For example, analysis results of the sensor module unit 210 are used to perform processing mainly related to perception. For example, perceptual information such as “Dad is alone” and “There is a 90% probability that dad is not smiling” is generated. A process of understanding the meaning of the generated perceptual information is performed. For example, semantic information such as “Dad alone seems to be lonely” is generated.

The state recognition unit 230 recognizes the state of the robot 100 based on the information detected by the sensor unit 200. For example, the state recognition unit 230 recognizes the remaining battery level of the robot 100, the brightness of the surrounding environment of the robot 100, and the like as the states of the robot 100.

The emotion determination unit 232 determines an emotion value indicating the emotion of the user 10 based on the information analyzed by the sensor module unit 210 and the state of the user 10 recognized by the state recognition unit 230. For example, the information analyzed by the sensor module unit 210 and the recognized state of the user 10 are input to a pre-trained neural network to acquire an emotion value indicating the emotion of the user 10.

Here, the emotion value indicating the emotion of the user 10 is a value indicating whether the emotion of the user is positive or negative. For example, if the emotion of the user is a bright emotion accompanied with pleasure or comfort, such as “joy”, “pleasure”, “comfort”, “relief”, “excitement”, “reassurance”, and “fulfillment”, a positive value is indicated, and the value becomes greater as the emotion is brighter. If the user's emotion is an emotion that makes the user feel unpleasant, such as “anger”, “sorrow”, “discomfort”, “anxiety”, “sadness”, “worry”, and “emptiness”, a negative value is indicated, and the absolute value of the negative value increases as the user feels unpleasant. In a case in which the user's emotion is not any of the above (“neutral”), the value 0 is indicated.

Furthermore, the emotion determination unit 232 determines an emotion value indicating the emotion of the robot 100 based on the information analyzed by the sensor module unit 210, the information detected by the sensor unit 200, and the state of the user 10 recognized by the state recognition unit 230.

The emotion value of the robot 100 includes the emotion value for each of multiple emotion classifications, and is, for example, a value (0 to 5) indicating the intensity of each of “joy”, “anger”, “sorrow”, and “pleasure”.

Specifically, the emotion determination unit 232 determines an emotion value indicating the emotion of the robot 100 according to a rule for updating the emotion value of the robot 100 defined in association with the information analyzed by the sensor module unit 210 and the state of the user 10 recognized by the state recognition unit 230.

For example, in a case in which the state recognition unit 230 recognizes that the user 10 seems to be lonely, the emotion determination unit 232 increases the emotion value for “sorrow” of the robot 100. Furthermore, in a case in which the state recognition unit 230 recognizes that the user 10 has a smiling face, the emotion value for “joy” of the robot 100 is increased.

Note that the emotion determination unit 232 may determine the emotion value indicating the emotion of the robot 100 in further consideration of the state of the robot 100. For example, in a case in which the remaining battery level of the robot 100 is low, a case in which the surrounding environment of the robot 100 is completely dark, or the like, the emotion value for “sorrow” of the robot 100 may be increased. Furthermore, the emotion value for “anger” may be increased in a case in which the user 10 continuously talks even though the remaining battery level is low.

The action recognition unit 234 recognizes an action of the user 10 based on the information analyzed by the sensor module unit 210 and the state of the user 10 recognized by the state recognition unit 230. For example, the information analyzed by the sensor module unit 210 and the recognized state of the user 10 are input to a pre-trained neural network, the probability of each of multiple predetermined action classifications (for example, “smile”, “getting angry”, “asking”, and “getting sad”) is acquired, and the action classification having the highest probability is recognized as the action of the user 10.

As described above, in the present embodiment, the robot 100 acquires the utterance content of the user 10 after identifying the user 10, but in acquiring and using the utterance content, the action control system of the robot 100 according to the present embodiment considers protection of personal information and privacy of the user 10 in addition to acquiring necessary consent from the user 10 according to laws and regulations.

Next, processing of the action determination unit 236 when the robot 100 performs a response process in which the robot responds to the action of the user 10 will be described.

The action determination unit 236 determines an action corresponding to the action of the user 10 recognized by the action recognition unit 234 based on the current emotion value of the user 10 determined by the emotion determination unit 232, the history data 222 of the past emotion values determined by the emotion determination unit 232 before the current emotion value of the user 10 is determined, and the emotion value of the robot 100. In the present embodiment, a case in which the action determination unit 236 uses one most recent emotion value included in the history data 222 as a past emotion value of the user 10 will be described, but the disclosed technology is not limited to this aspect. For example, the action determination unit 236 may use multiple most recent emotion values as the past emotion values of the user 10, or may use emotion values that are earlier by a unit period such as one day earlier. Furthermore, the action determination unit 236 may determine an action corresponding to the action of the user 10 in further consideration of the history of the past emotion values of the robot 100 in addition to the current emotion value of the robot 100. The action determined by the action determination unit 236 includes a gesture performed by the robot 100 or utterance content of the robot 100.

The action determination unit 236 according to the present embodiment determines an action of the robot 100 based on a combination of the past emotion value and the current emotion value of the user 10, the emotion value of the robot 100, the action of the user 10, and the action determination model 221 as an action corresponding to the action of the user 10. For example, in a case in which the past emotion value of the user 10 is a positive value and the current emotion value is a negative value, the action determination unit 236 determines an action for positively changing the emotion value of the user 10 as an action corresponding to the action of the user 10.

In the reaction rules as the action determination model 221, an action of the robot 100 according to the combination of the past emotion value and the current emotion value of the user 10, the emotion value of the robot 100, and the action of the user 10 is determined. For example, in a case in which the past emotion value of the user 10 is a positive value, the current emotion value is a negative value, and the action of the user 10 is “getting sad”, a combination of the gesture and utterance content of making an inquiry to encourage the user 10 with a gesture is determined as the action of the robot 100.

For example, in the reaction rules as the action determination model 221, the action of the robot 100 is determined for all combinations of the pattern of the emotion value of the robot 100 (1296 patterns that is the fourth power of six values of “joy”, “anger”, “sorrow”, and “pleasure” values from “0” to “5”), the pattern of the combinations of the past emotion value and the current emotion value of the user 10, and the action pattern of the user 10. That is, for each pattern of the emotion value of the robot 100, the action of the robot 100 according to the action pattern of the user 10 is determined for each of multiple combinations such that the combinations of the past emotion value and the current emotion value of the user 10 are a negative value and a negative value, a negative value and a positive value, a positive value and a negative value, a positive value and a positive value, a negative value and a neutral value, and a neutral value and a neutral value. Note that the action determination unit 236 may transition to the operation mode of determining the action of the robot 100 using the history data 222, for example, in a case in which the user 10 makes an utterance intending to continue a conversation over a past topic, such as saying “I want to talk about that topic we discussed before”.

Note that, in the reaction rules as the action determination model 221, at least one of a gesture or the utterance content may be determined as the action of the robot 100 for each of the patterns (1296 patterns) of the emotion values of the robot 100 at the maximum. Alternatively, in the reaction rules as the action determination model 221, at least one of the gesture or the utterance content may be determined as the action of the robot 100 for each of the groups of the patterns of the emotion values of the robot 100.

The intensity of each gesture included in the action of the robot 100 defined in the reaction rules as the action determination model 221 is determined in advance. In each utterance content included in the action of the robot 100 defined in the reaction rules as the action determination model 221, the intensity of the utterance content is determined in advance.

The memory control unit 238 determines whether or not to store data including the action of the user 10 in the history data 222 based on the intensity of the action determined in advance for the action determined by the action determination unit 236 and the emotion value of the robot 100 determined by the emotion determination unit 232.

Specifically, in a case in which the total value of the sum of the emotion values for each of the multiple emotion classifications of the robot 100 and the intensity that is the sum of the intensity predetermined for the gesture included in the action determined by the action determination unit 236 and the intensity predetermined for the utterance content included in the action determined by the action determination unit 236 is a threshold value or greater, it is determined to store data including the action of the user 10 in the history data 222.

In a case in which it is determined to store the data including the action of the user 10 in the history data 222, the action determined by the memory control unit 238 stores, in the history data 222, the action determined by the action determination unit 236, the information (for example, all peripheral information such as data of a sound, an image, and a smell of the place) analyzed by the sensor module unit 210 from the current time point to a certain period before, and the state of the user 10 (for example, the expression, emotion, and the like of the user 10) recognized by the state recognition unit 230.

The action control unit 250 controls the control target 252 based on the action determined by the action determination unit 236. For example, in a case in which the action determination unit 236 determines an action including utterance, the action control unit 250 causes a speaker included in the control target 252 to output a voice. At this time, the action control unit 250 may determine the speed of the voice uttered based on the emotion value of the robot 100. For example, the action control unit 250 determines a higher utterance speed as the emotion value of the robot 100 is larger. In this manner, the action control unit 250 determines the execution form of the action determined by the action determination unit 236 based on the emotion value determined by the emotion determination unit 232.

The action control unit 250 may recognize a change in emotion of the user 10 with respect to execution of the action determined by the action determination unit 236. For example, the change in the emotion of the user 10 may be recognized based on the voice or expression of the user 10. In addition, the change in emotion of the user 10 may be recognized based on the detection of an impact by the touch sensor 205 included in the sensor unit 200. In a case in which an impact is detected by the touch sensor 205 included in the sensor unit 200, it may be recognized that the emotion of the user 10 has been worsened, or in a case in which it is determined that the reaction of the user 10 is smiling or joyful from the detection result of the touch sensor 205 included in the sensor unit 200, it may be recognized that the emotion of the user 10 has got better. Information indicating the reaction of the user 10 is output to the communication processing unit 280.

Furthermore, after the action control unit 250 executes the action determined by the action determination unit 236 in the execution mode determined according to the emotion of the robot 100, the emotion determination unit 232 further changes the emotion value of the robot 100 based on the user's reaction to the execution of the action. Specifically, the emotion determination unit 232 increases the emotion value for “joy” of the robot 100 in a case in which the user's reaction to the action determined by the action determination unit 236, performed on the user in the execution mode determined by the action control unit 250, is not unfavorable. Specifically, the emotion determination unit 232 increases the emotion value for “sorrow” of the robot 100 in a case in which the user's reaction to the action determined by the action determination unit 236, performed on the user in the execution mode determined by the action control unit 250, is unfavorable.

Furthermore, the action control unit 250 expresses the emotion of the robot 100 based on the determined emotion value of the robot 100. For example, in a case in which the emotion value for “joy” of the robot 100 is increased, the action control unit 250 controls the control target 252 to cause the robot 100 to perform a gesture of joy. Furthermore, in a case in which the emotion value for “sorrow” of the robot 100 is increased, the action control unit 250 controls the control target 252 such that the posture of the robot 100 is a dejected posture.

The communication processing unit 280 is responsible for communication with the server 300. As described above, the communication processing unit 280 transmits user reaction information to the server 300. Furthermore, the communication processing unit 280 receives an updated reaction rule from the server 300. Upon receiving the updated reaction rule from the server 300, the communication processing unit 280 updates the reaction rule as the action determination model 221.

The server 300 performs communication between the robot 100, the robot 101, and the robot 102 and the server 300, receives the user reaction information transmitted from the robot 100, and updates the reaction rule based on the reaction rule including the action for which a positive reaction has been obtained.

The related information collection unit 270 collects information related to preference information from external data (web sites such as news sites and moving image sites) based on the preference information acquired for the user 10 at a predetermined timing.

Specifically, the related information collection unit 270 acquires preference information indicating a matter of interest of the user 10 from utterance content of the user 10 or a setting operation by the user 10 in advance. The related information collection unit 270 collects news related to the preference information from external data at regular intervals using, for example, ChatGPT Plugins (retrieved from the Internet <URL: https://openai.com/blog/chatgpt-plugins>). For example, in a case in which it is acquired as preference information that the user 10 is a fan of a specific professional baseball team, the related information collection unit 270 collects news related to the game result of the specific professional baseball team from external data at a predetermined time every day, for example, using ChatGPT Plugins.

The emotion determination unit 232 determines the emotion of the robot 100 based on the information related to the preference information collected by the related information collection unit 270.

Specifically, the emotion determination unit 232 inputs a text indicating the information related to the preference information collected by the related information collection unit 270 to a pre-trained neural network for determining an emotion, acquires the emotion value indicating each emotion, and determines the emotion of the robot 100. For example, in a case in which the collected news related to the game result of the specific professional baseball team indicates that the specific professional baseball team has won, the emotion value for “joy” of the robot 100 is determined to be high.

In a case in which the emotion value of the robot 100 is a threshold value or greater, the memory control unit 238 stores information related to the preference information collected by the related information collection unit 270 in the collected data 223.

Next, processing of the action determination unit 236 when the robot 100 performs an autonomous process for autonomous acting will be described.

In the autonomous processing in the present embodiment, the robot 100 dreams. That is, an original event is created.

The action determination unit 236 uses at least one of the state of the user 10, the emotion of the user 10, the emotion of the robot 100, or the state of the robot 100, together with the action determination model 221 at a predetermined timing, to determine, as the action of the robot 100, any of multiple types of robot actions, including not acting. Here, a case in which a sentence generation model having an interaction function is used as the action determination model 221 will be described as an example.

Specifically, the action determination unit 236 inputs a text representing at least one of the state of the user 10, the emotion of the user 10, the emotion of the robot 100, or the state of the robot 100, together with a text for asking about the robot action to the sentence generation model to determine the action of the robot 100 based on the output of the sentence generation model.

For example, multiple types of the robot actions include the following (1) to (10).
(1) The robot does nothing.
(2) The robot dreams.
(3) The robot speaks to the user.
(4) The robot creates a picture diary.
(5) The robot proposes an activity.
(6) The robot proposes a person whom the user should meet.
(7) The robot introduces news that the user is interested in.
(8) The robot edits pictures and videos.
(9) The robot studies with the user.
(10) The robot evokes a memory.

The action determination unit 236 inputs, to the sentence generation model, a text indicating the state of the user 10 and the state of the robot 100 recognized by the state recognition unit 230, the current emotion value of the user 10 determined by the emotion determination unit 232, and the current emotion value of the robot 100, and a text for asking about any of multiple types of robot actions including not acting every time of a certain period of time elapses, and determines the action of the robot 100 based on the output of the sentence generation model. Here, in a case in which there is no user 10 around the robot 100, the text to be input to the sentence generation model need not include the state of the user 10 and the current emotion value of the user 10, or may include the fact that there is no user 10.

The sentence generation model receives an input of a text “The robot is in a very pleasant state. The user is normally in a pleasant state. The user is sleeping. Which one of the following (1) to (10) is better as an action of the robot?
(1) The robot does nothing.
(2) The robot dreams.
(3) The robot speaks to the user.
. . . ” as another example. Based on the output “It can be said that either (1) The robot does nothing or (2) The robot dreams is the most appropriate action” of the sentence generation model, “(1) The robot does nothing” or “(2) The robot dreams” is determined as an action of the robot 100.

The sentence generation model receives an input of a text “The robot is in a slightly sad state. The user is absent. It is dark around the robot. Which one of the following (1) to (10) is better as an action of the robot? (1) The robot does nothing.
(2) The robot dreams.
(3) The robot speaks to the user.
. . . ” as another example. Based on the output “It can be said that either (2) The robot dreams or (4) The robot creates a picture diary is the most appropriate action” of the sentence generation model, “(2) The robot dreams” or “(4) The robot creates a picture diary” is determined as an action of the robot 100.

In a case in which the action determination unit 236 determines that “(2) The robot dreams”, that is, creation of an original event, as a robot action, the action determination unit creates the original event obtained by combining multiple pieces of event data in the history data 222 using the sentence generation model. At this time, the memory control unit 238 stores the created original event in the history data 222.

At this time, the action determination unit 236 creates the original event while randomly shuffling or exaggerating the past experience and conversation between the robot 100 and the user 10 or the family of the user 10 in the history data 222. Furthermore, based on the created original event, that is, the dream, the image generation model may be used to generate a dream image presented as a collage. In this case, a dream image may be generated based on one scene from the past memory stored in the history data 222, or a plurality of memories may be randomly shuffled and combined to generate a dream image. Furthermore, not only the dream image may represent what has not actually occurred such as a “dream” but also an image representing what the robot 100 has seen and heard while the user 10 is not present may be generated as a dream image. The generated dream image is, so to speak, a dream diary. At this time, by using crayons for touches on the dream image, a more dream-like atmosphere can be given to the image. Then, the action determination unit 236 stores the action of outputting the generated dream image in the action plan data 224. As a result, according to the action plan data 224, the robot 100 can take an action of outputting the generated dream image to the display or transmitting the generated dream image to the terminal of the user.

Note that the action determination unit 236 may cause the robot 100 to output a voice based on the original event. For example, in a case in which the original event relates to a panda, an utterance like “I dreamed of a panda. Take me to the zoo.” to be made in the next morning may be stored in the action plan data 224. Furthermore, also in this case, in addition to uttering a thing that has not actually occurred, such as a “dream”, the robot 100 may utter, as an experience episode of the robot itself, what the robot 100 has seen and heard while the user 10 is not present.

In a case in which it is determined that “(3) The robot speaks to the user”, that is, the robot 100 utters, as a robot action, the action determination unit 236 determines the utterance content of the robot corresponding to the user state and the user's emotion or the robot's emotion using the sentence generation model. At this time, the action control unit 250 causes a speaker included in the control target 252 to output a voice representing the determined utterance content of the robot. Note that, in a case in which the user 10 is absent around the robot 100, the action control unit 250 stores the determined utterance content of the robot in the action plan data 224 without outputting a voice representing the determined utterance content of the robot.

In a case in which it is determined that “(7) The robot introduces news that the user is interested in” as a robot action, the action determination unit 236 determines the utterance content of the robot corresponding to the information stored in the collected data 223 using the sentence generation model. At this time, the action control unit 250 causes a speaker included in the control target 252 to output a voice representing the determined utterance content of the robot. Note that, in a case in which the user 10 is absent around the robot 100, the action control unit 250 stores the determined utterance content of the robot in the action plan data 224 without outputting a voice representing the determined utterance content of the robot.

In a case in which it is determined that “(4) The robot creates a picture diary”, that is, the robot 100 creates an event image, as a robot action, the action determination unit 236 generates an image representing the event data for the event data selected from the history data 222 using an image generation model, generates an explanatory sentence representing the event data using the sentence generation model, and outputs a combination of the image representing the event data and the explanatory sentence representing the event data as an event image. Note that, in a case in which the user 10 is absent around the robot 100, the action control unit 250 stores the event image in the action plan data 224 without outputting the event image.

In a case in which it is determined that “(8) The robot edits pictures and videos”, that is, the robot edits images, the action determination unit 236 selects event data from the history data 222 based on the emotion value, edits the image data of the selected event data, and outputs the edited image data. Note that, in a case in which the user 10 is absent around the robot 100, the action control unit 250 stores the edited image data in the action plan data 224 without outputting the edited image data.

In a case in which it is determined that “(5) The robot proposes an activity”, that is, an action of the user 10 is proposed, as a robot action, the action determination unit 236 determines the proposed action of the user using the sentence generation model based on the event data stored in the history data 222. At this time, the action control unit 250 causes a speaker included in the control target 252 to output a voice proposing the action of the user. Note that, in a case in which the user 10 is absent around the robot 100, the action control unit 250 stores the proposal on the action of the user in the action plan data 224 without outputting a voice proposing the action of the user.

In a case in which it is determined, as a robot action, that “(6) The robot proposes a person whom the user should meet”, that is, the robot proposes a partner who should be engaged with the user 10, the action determination unit 236 determines the proposed partner who should be engaged with the user using the sentence generation model based on the event data stored in the history data 222. At this time, the action control unit 250 causes a speaker included in the control target 252 to output a voice proposing the partner who should be engaged with the user. Note that, in a case in which the user 10 is absent around the robot 100, the action control unit 250 stores the proposal on the partner who should be engaged with the user in the action plan data 224 without outputting a voice indicating the proposal on the partner who should be engaged with the user.

In a case in which it is determined that “(9) The robot studies with the user”, that is, the robot 100 utters about studying as a robot action, the action determination unit 236 determines the utterance content of the robot for encouraging studying, presenting study problems, or giving advice related to studying corresponding to the user state and the user's emotion or the robot's emotion using the sentence generation model. At this time, the action control unit 250 causes a speaker included in the control target 252 to output a voice representing the determined utterance content of the robot. Note that, in a case in which the user 10 is absent around the robot 100, the action control unit 250 stores the determined utterance content of the robot in the action plan data 224 without outputting a voice representing the determined utterance content of the robot.

In a case in which it is determined, as a robot action, that “(10) The robot evokes memory”, that is, the robot remembers the event data, the action determination unit 236 selects the event data from the history data 222. At this time, the emotion determination unit 232 determines the emotion of the robot 100 based on the selected event data. Furthermore, the action determination unit 236 creates an emotion change event representing the utterance content or action of the robot 100 for changing the emotion value of the user using the sentence generation model based on the selected event data. At this time, the memory control unit 238 stores the emotion change event in the action plan data 224.

For example, in a case in which it is stored in the history data 222 that the video the user was watching was related to a panda as event data, and the event data is selected, a message like “What would you say about the topic related to a panda when you meet the user next time? Take three examples” is input to the sentence generation model. In a case in which the output of the sentence generation model is “(1) Let's go to the zoo; (2) draw a picture of a panda; and (3) let's go buy a stuffed panda doll”, the robot 100 inputs “What makes the user most happy among (1), (2), and (3)?” to the sentence generation model. In a case in which the output of the sentence generation model is “(1) Let's go to the zoo”, the robot 100 creates uttering “(1) Let's go to the zoo” when the robot 100 meets the user next time, as an emotion change event, and stores the emotion change event in the action plan data 224.

Furthermore, for example, event data having a large emotion value of the robot 100 is selected as an impressive memory of the robot 100. This makes it possible to create an emotion change event based on the event data selected as an impressive memory.

Based on the state of the user 10 recognized by the state recognition unit 230, in a case in which an action of the user 10 with respect to the robot 100 is detected in a state where there is no action of the user 10 with respect to the robot 100, the action determination unit 236 reads data stored in the action plan data 224 and determines an action of the robot 100.

For example, in a case in which the user 10 is absent around the robot 100 but the user 10 is detected, the action determination unit 236 reads data stored in the action plan data 224 and determines an action of the robot 100. In addition, when it is detected that the user 10 has woken up in a case in which the user 10 was sleeping, the action determination unit 236 reads data stored in the action plan data 224 and determines an action of the robot 100.

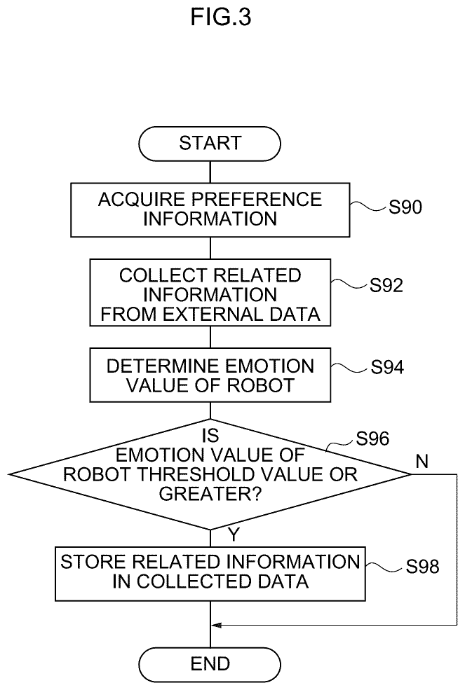

FIG. 3 schematically shows an example of an operation flow related to a collection process of collecting information related to preference information of the user 10. The operation flow shown in FIG. 3 is repeatedly executed in every certain period. It is assumed that preference information indicating a matter of interest to the user 10 has been acquired from the utterance content of the user 10 or the setting operation by the user 10. Note that “S” in the operation flow represents a step to be executed.

First, in step S90, the related information collection unit 270 acquires preference information indicating a matter of interest to the user 10.

In step S92, the related information collection unit 270 collects information related to the preference information from external data.

In step S94, the emotion determination unit 232 determines the emotion value of the robot 100 based on the information related to the preference information collected by the related information collection unit 270.

In step S96, the memory control unit 238 determines whether or not the emotion value of the robot 100 determined in step S94 is a threshold value or greater. If the emotion value of the robot 100 is less than the threshold value, the information related to the collected preference information is not stored in the collected data 223, and the process ends. On the other hand, if the emotion value of the robot 100 is the threshold value or greater, the process proceeds to step S98.

In step S98, the memory control unit 238 stores the information related to the collected preference information in the collected data 223, and ends the process.

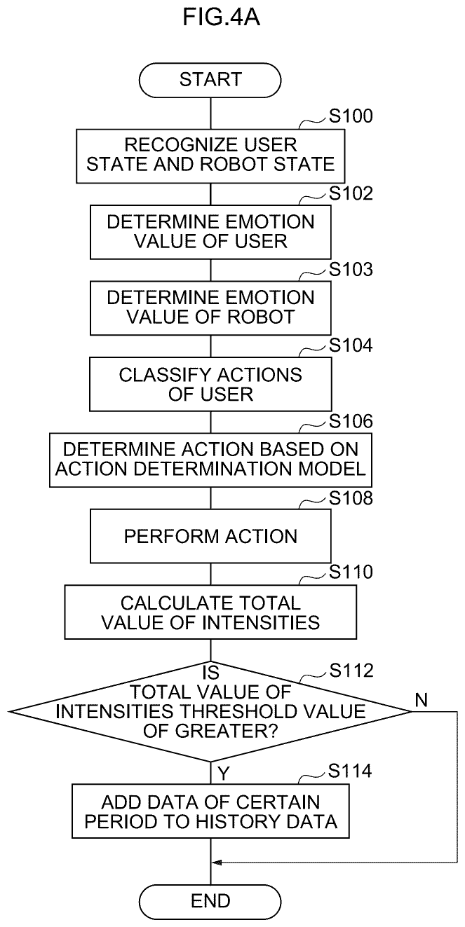

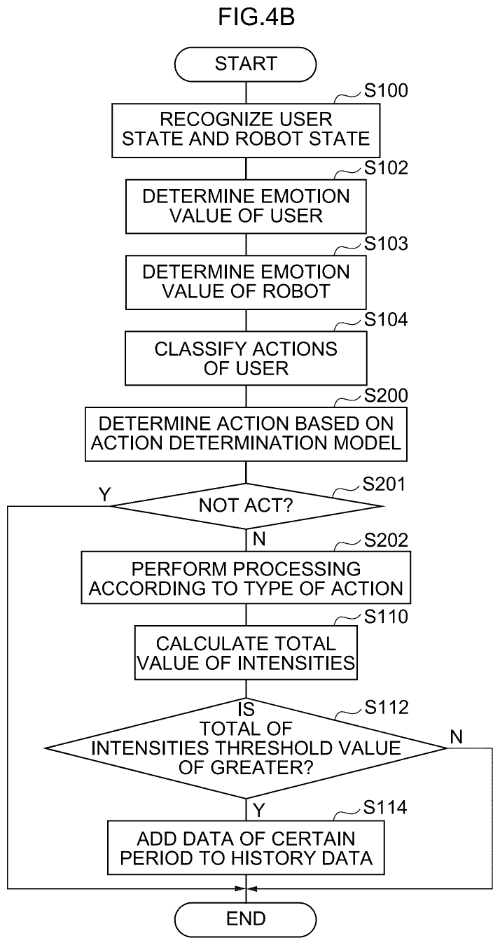

FIG. 4A schematically shows an example of the operation flow related to an operation of determining an action in the robot 100 when the robot 100 performs a response process in which the robot 100 responds to an action of the user 10. The operation flow shown in FIG. 4A is repeatedly executed. At this time, it is assumed that information analyzed by the sensor module unit 210 has been input.

First, in step S100, the state recognition unit 230 recognizes the state of the user 10 and the state of the robot 100 based on the information analyzed by the sensor module unit 210.

In step S102, the emotion determination unit 232 determines an emotion value indicating the emotion of the user 10 based on the information analyzed by the sensor module unit 210 and the state of the user 10 recognized by the state recognition unit 230.

In step S103, the emotion determination unit 232 determines an emotion value indicating the emotion of the robot 100 based on the information analyzed by the sensor module unit 210 and the state of the user 10 recognized by the state recognition unit 230. The emotion determination unit 232 adds the determined emotion value of the user 10 and emotion value of the robot 100 to the history data 222.

In step S104, the action recognition unit 234 recognizes the action classification of the user 10 based on the information analyzed by the sensor module unit 210 and the state of the user 10 recognized by the state recognition unit 230.

In step S106, the action determination unit 236 determines the action of the robot 100 based on a combination of the current emotion value of the user 10 determined in step S102 and the past emotion value included in the history data 222, the emotion value of the robot 100, the action of the user 10 recognized in step S104, and the action determination model 221.

In step S108, the action control unit 250 controls the control target 252 based on the action determined by the action determination unit 236.

In step S110, the memory control unit 238 calculates the total value of the intensities based on the intensity of the action predetermined for the action determined by the action determination unit 236 and the emotion value of the robot 100 determined by the emotion determination unit 232.

In step S112, the memory control unit 238 determines whether or not the total value of the intensities is a threshold value or greater. If the total value of the intensities is less than the threshold value, the event data including the action of the user 10 is not stored in the history data 222, and the process ends. On the other hand, if the total value of the intensities is the threshold value or greater, the process proceeds to step S114.

In step S114, event data including the action determined by the action determination unit 236, the information analyzed by the sensor module unit 210 from the current time point to a certain period before, and the state of the user 10 recognized by the state recognition unit 230 are stored in the history data 222.

FIG. 4B schematically shows an example of the operation flow related to an operation of determining an action in the robot 100 when the robot 100 performs an autonomous process for autonomous acting. The operation flow shown in FIG. 4B is repeatedly and automatically executed, for example, each time a certain time elapses. At this time, it is assumed that information analyzed by the sensor module unit 210 has been input. Note that, in the same process as FIG. 4A described above, the same step numbers are indicated.

First, in step S100, the state recognition unit 230 recognizes the state of the user 10 and the state of the robot 100 based on the information analyzed by the sensor module unit 210.

In step S102, the emotion determination unit 232 determines an emotion value indicating the emotion of the user 10 based on the information analyzed by the sensor module unit 210 and the state of the user 10 recognized by the state recognition unit 230.

In step S103, the emotion determination unit 232 determines an emotion value indicating the emotion of the robot 100 based on the information analyzed by the sensor module unit 210 and the state of the user 10 recognized by the state recognition unit 230. The emotion determination unit 232 adds the determined emotion value of the user 10 and emotion value of the robot 100 to the history data 222.

In step S104, the action recognition unit 234 recognizes the action classification of the user 10 based on the information analyzed by the sensor module unit 210 and the state of the user 10 recognized by the state recognition unit 230.

In step S200, the action determination unit 236 determines, as an action of the robot 100, any of multiple types of robot actions including not acting based on the state of the user 10 recognized in step S100, the emotion of the user 10 determined in step S102, the emotion of the robot 100, the state of the robot 100 recognized in step S100, the action of the user 10 recognized in step S104, and the action determination model 221.

In step S201, the action determination unit 236 determines whether not acting is determined in step S200. If not acting is determined as an action of the robot 100, the process ends. On the other hand, if not acting is not determined as an action of the robot 100, the process proceeds to step S202.

In step S202, the action determination unit 236 performs processing according to the type of the robot action determined in step S200 described above. At this time, the action control unit 250, the emotion determination unit 232, or the memory control unit 238 executes processing in accordance with the type of the robot action.

In step S110, the memory control unit 238 calculates the total value of the intensities based on the intensity of the action predetermined for the action determined by the action determination unit 236 and the emotion value of the robot 100 determined by the emotion determination unit 232.

In step S112, the memory control unit 238 determines whether or not the total value of the intensities is a threshold value or greater. If the total value of the intensities is less than the threshold value, the data including the action of the user 10 is not stored in the history data 222, and the process ends. On the other hand, if the total value of the intensities is the threshold value or greater, the process proceeds to step S114.

In step S114, the memory control unit 238 stores, in the history data 222, the action determined by the action determination unit 236, the information analyzed by the sensor module unit 210 from the current time point to a certain period before, and the state of the user 10 recognized by the state recognition unit 230.

As described above, according to the robot 100, the emotion value indicating the emotion of the robot 100 is determined based on the user state, and whether or not to store data including the action of the user 10 in the history data 222 is determined based on the emotion value of the robot 100. As a result, the capacity of the history data 222 that stores data including the action of the user 10 can be reduced Then, for example, in a case in which the robot 100 determines that the user state is the same as the user state was 10 years ago after 10 years, the robot 100 reads the history data 222 of 10 years ago, and thus, can present the state of the user 10 of 10 years ago (for example, the expression, emotion, and the like of the user 10), and further, any peripheral information such as data of the voice, image, scent, and the like of the place to the user 10.

Furthermore, according to the robot 100, it is possible to cause the robot 100 to execute an appropriate action in response to the action of the user 10. In the related art, actions of a user are classified, and an action including an expression or an appearance of a robot is determined. With regard to this, the robot 100 determines the current emotion value of the user 10 and executes an action on the user 10 based on the past emotion value and the current emotion value. Therefore, for example, in a case in which the user 10 was fine yesterday but is depressed today, the robot 100 can utter the following: “You were fine yesterday. What's wrong with you today?”. Furthermore, the robot 100 can also perform an utterance with gestures. Furthermore, for example, in a case in which the user 10 was depressed yesterday but is fine today, the robot 100 can utter the following: “You were depressed yesterday, but you look fine today!”. Furthermore, for example, in a case in which the user 10 was fine yesterday and is better today than yesterday, the robot 100 can utter the following: “You look better today than yesterday. What made you better than yesterday?”. Furthermore, for example, the robot 100 can utter the following to the user 10 whose emotion value is 0 or higher and whose state in which the fluctuation range of the emotion value is within a certain range: “Recently, you seem to be stable, which is good”.

Furthermore, for example, in a case in which the robot 100 asks “Did you finish the assignment you mentioned yesterday?” to the user 10 and receives the answer “I did it” from the user 10, the robot can make an affirmative utterance such as “Good!” and make an affirmative gesture such as applause or thumbs-up. Furthermore, for example, when the user 10 utters “The presentation we discussed the day before yesterday was successful”, the robot 100 can make an affirmative utterance such as “Good job!” and also make the above affirmative gesture. As described above, the robot 100 performs an action based on the history of the state of the user 10, and thereby it is expected that the user 10 can feel a sense of closeness to the robot 100.

Furthermore, for example, in a case in which the emotion value of “pleasure” of the emotion of the user 10 is a threshold value or higher when the user 10 is watching a video related to pandas, the appearance scene of a panda in the video may be stored in the history data 222 as event data.

Using the data accumulated in the history data 222 and the collected data 223, the robot 100 can always learn in what conversation the user has a maximum emotion value expressing that the user is happy.

Furthermore, in a state in which the robot 100 is not in conversation with the user 10, it is possible to autonomously start an action based on the emotion of the robot 100.

Furthermore, in the autonomous process, the robot 100 repeats automatically generating a question, inputting the question to the sentence generation model, and acquiring an output of the sentence generation model as the answer to the question, so that it is possible to create an emotion change event for boosting a good emotion and store the emotion change event in the action plan data 224. In this manner, the robot 100 can execute self-learning.

Furthermore, when the robot 100 automatically generates a question without receiving a trigger from the outside, the question can be automatically generated based on event data remaining in an impression specified from a history of past emotion values of the robot.

Furthermore, the related information collection unit 270 can execute self-learning by repeating a search execution stage in which keyword search is automatically performed in accordance with the preference information of the user to acquire a search result.

Here, in the search execution stage, the keyword search may be automatically executed based on the event data remaining the impression specified from the history of the past emotion values of the robot while no trigger is received from the outside.

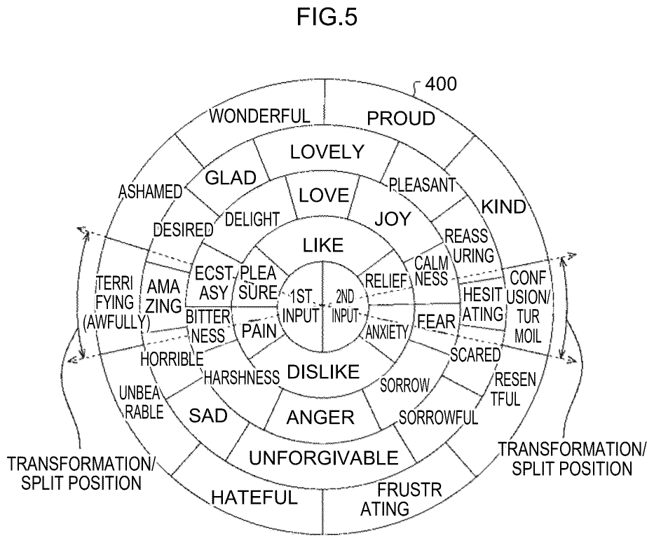

Note that the emotion determination unit 232 may determine the user's emotion according to specific mapping. Specifically, the emotion determination unit 232 may determine the user's emotion based on an emotion map (see FIG. 5) that is specific mapping.

FIG. 5 is a diagram illustrating an emotion map 400 on which multiple emotions are mapped. In the emotion map 400, emotions are arranged concentrically radially from the center. The closer to the center of the concentric circles, the more the emotion in the primitive state is arranged. Emotions indicating states and actions generated from the state of mind are arranged outside the concentric circles. An emotion is a concept including feelings and mental states. On the left side of the concentric circles, emotions generated from reactions generally occurring in the brain are arranged. On the right side of the concentric circles, emotions induced by situation judgment are generally arranged. In the upward and downward directions of the concentric circles, emotions generated from reactions generally occurring in the brain and induced by situation judgment are arranged. Furthermore, the emotion “pleasure” is arranged on the upper side of the concentric circles, and the emotion “discomfort” is arranged on the lower side. As described above, in the emotion map 400, multiple emotions are mapped based on a structure in which emotions are generated, and emotions that are likely to occur at the same time are mapped close to each other.

(1) For example, in a case in which the emotion engine, which is the emotion determination unit 232 of the robot 100, detects emotions at about 100 msec, the determination of the reaction operation (for example, backchanneling) of the robot 100 may be set at a timing at which the frequency is at least similar to the detection frequency (100 msec) of the emotion engine even if the frequency is low, or may be set at a timing quicker than the detection frequency. The detection frequency of the emotion engine may be interpreted as a sampling rate.

The emotion is detected at about 100 msec, and the reaction operation (for example, backchanneling) is performed immediately in conjunction with the detection, whereby unnatural backchanneling is eliminated, and natural and context-aware interactions can be realized. The robot 100 performs a reaction operation (backchanneling or the like) according to the directionality and the degree (intensity) of the mandala of the emotion map 400. Note that the detection frequency (sampling rate) of the emotion engine is not limited to 100 ms, and may be changed according to the situation (such as when playing sports), the age of the user, or the like.

(2) In comparison with the emotion map 400, the directionality of the emotion and the intensity of the degree thereof may be preset, and the movement of the acknowledgement and the intensity of the acknowledgement may be set. For example, in a case in which the robot 100 feels a sense of stability, relief, or the like, the robot 100 continues listening to speech while nodding. In a case in which the robot 100 feels anxious, lost, or suspicious, the robot 100 may tilt its head or stop swinging.

These emotions are distributed in the 3 o'clock direction of the emotion map 400, and usually come and go between relief and anxiety. In the right half of the emotion map 400, situation recognition is superior to internal sensation, and thus gives a calm impression.

(3) In a case in which the robot 100 is experiencing pleasure after receiving compliments, a filler “Oh” may come in front of the line, and in a case in which the robot is experiencing pain after receiving harsh words, a filler “Ohh!” may come in front of the line. Furthermore, a physical reaction such as a gesture of the robot 100 crouching while saying “Ohh!” may be included. These emotions are distributed to around 9 o'clock direction in the emotion map 400.

(4) In the left half of the emotion map 400, internal sensation (reaction) is prioritized over situation recognition. Therefore, the impression of an unintentional reaction can be given.

In a case in which the robot 100 has a favorable feeling in situation recognition while having an internal feeling (reaction) of conviction, the robot 100 may nod deeply while looking at the partner, or may utter “yeah”. In this manner, the robot 100 may generate a balanced favorable feeling for the partner, that is, an action such as accepting or understanding for the partner. These emotions are distributed to around 12 o'clock direction in the emotion map 400.

On the other hand, even in the situation recognition while the robot 100 has the internal feeling (reaction) of discomfort, the robot 100 may shake its head sideways when feeling antipathy, and may turn the LEDs of the eyes red and look at the partner when feeling hatred. These emotions are distributed around 6 o'clock in the emotion map 400.

(5) Since the inside of the emotion map 400 represents the inside of the mind and the outside of the emotion map 400 represents an action, the emotion is more visible (appears in the action) toward the outside of the emotion map 400.

(6) In a case in which the robot 100 listens to a person's speech while feeling the sense of relief distributed around 3 o'clock in the emotion map 400, the robot slightly shakes its head vertically saying “Hun Hun”; however, in the direction of love around 12 o'clock, the robot may perform strong nodding such as deeply moving its head vertically.

Here, human emotions are based on various balances such as posture and blood glucose level, and indicate a state of discomfort when the balance goes away from the ideal level and a state of comfort when the balance approaches the ideal level. Even in a robot, an automobile, a motorcycle, or the like, based on various balances such as a posture and a remaining battery level, it is possible to make emotions so as to indicate a state of discomfort when the balance goes away from the ideal level and a state of comfort when the balance approaches the ideal level. The emotion map may be generated, for example, based on an emotion map (Research on the phonetic recognition of feelings and a system for emotional physiological brain signal analysis, Tokushima University, PhD thesis: https://ci.nii.ac.jp/naid/500000375379) of Dr. Mitsuyoshi. In the left half of the emotion map, emotions belonging to a region called “reaction” in which sensations are superior are arranged. Furthermore, in the right half of the emotion map, emotions belonging to a region called “situation” in which situation recognition is superior are arranged.

In the emotion map, two emotions emotion encouraging learning are defined. One is an emotion around the core of negative “repentance” or “remorse” situated on the situation side. That is, it is when a negative emotion such as “I do not want to feel this again” or “I do not want to be reprimanded” occurs in the robot. The other emotion is one close to the positive “desire” situated on the reactive side. That is, it is the time of a positive feeling such as “desire more” or “want to know more”.

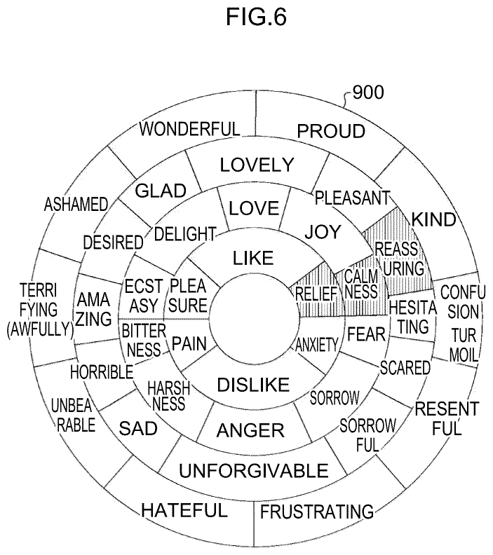

The emotion determination unit 232 inputs the information analyzed by the sensor module unit 210 and the recognized state of the user 10 to a pre-trained neural network, acquires an emotion value indicating each emotion indicated on the emotion map 400, and determines the emotion of the user 10. This neural network is pre-trained based on multiple pieces of learning data that is a combination of the information analyzed by the sensor module unit 210, the recognized state of the user 10, and the emotion value indicating each emotion indicated on the emotion map 400. Furthermore, in this neural network, as on an emotion map 900 illustrated in FIG. 6, it is trained that emotions arranged close to each other have close values. FIG. 6 illustrates an example in which multiple emotions such as “relief”, “calm”, and “reassuring” have similar emotion values.

Furthermore, the emotion determination unit 232 may determine the emotion of the robot 100 according to a specific mapping. Specifically, the emotion determination unit 232 inputs the information analyzed by the sensor module unit 210, the state of the user 10 recognized by the state recognition unit 230, and the state of the robot 100 to the pre-trained neural network, acquires an emotion value indicating each emotion indicated in the emotion map 400, and determines the emotion of the robot 100. This neural network is pre-trained based on multiple pieces of learning data that is a combination of the information analyzed by the sensor module unit 210, the recognized state of the user 10, the emotion of the robot 100, and the emotion value indicating each emotion indicated on the emotion map 400. For example, the neural network is trained based on training data indicating that the emotion value “3” for “joyful” is obtained in a case in which the robot 100 is recognized as being cared by the user 10 from the output of the touch sensor (not illustrated), and training data indicating that the emotion value “3” for “anger” is obtained in a case in which the robot 100 is recognized as being hit by the user 10 from the output of the acceleration sensor 206. Furthermore, in this neural network, as on an emotion map 900 illustrated in FIG. 6, it is trained that emotions arranged close to each other have close values.

The action determination unit 236 adds a fixed sentence for asking about the action content of the robot corresponding to an action of the user to the text representing the action of the user, the emotion of the user, and the emotion of the robot, and inputs the text to the sentence generation model having the interaction function, thereby generating the action content of the robot.

For example, the action determination unit 236 acquires a text indicating the state of the robot 100 from the emotion of the robot 100 determined by the emotion determination unit 232 using the emotion table as shown in Table 1. Here, in the emotion table, an index number is assigned to each emotion value for each type of emotion, and a text indicating the state of the robot 100 is stored for each index number.

In a case in which the emotion of the robot 100 determined by the emotion determination unit 232 corresponds to the index number “2”, a text “very pleasant state” is obtained. Note that, in a case in which the emotion of the robot 100 corresponds to multiple index numbers, multiple texts indicating the state of the robot 100 are obtained.

Furthermore, an emotion table as shown in Table 2 is prepared for emotions of the user 10.

Here, in a case in which the action of the user is to talk “Let's play together”, the emotion of the robot 100 is the index number “2”, and the emotion of the user 10 is the index number “3”, a text indicating “The robot is in a very pleasant state. The user is normally in a pleasant state. The user said “Let's play together” Then, how do I answer to that as a robot?” is input to the sentence generation model to acquire the action content of the robot. The action determination unit 236 determines an action of the robot from the action content.

TABLE 1

| Index | | Emotion | |
|---|---|---|---|
| number | Type of emotion | value | State of robot |
| 1 | Pleasant | 5 | Extremely pleasant state |
| 2 | Pleasant | 4 | Very pleasant state |
| 3 | Pleasant | 3 | Moderately pleasant state |
| 4 | Pleasant | 2 | Slightly pleasant state |
| 5 | Pleasant | 1 | Barely pleasant state |
| . . . | . . . | . . . | . . . |

TABLE 2

| Index | | Emotion | |
|---|---|---|---|
| number | Type of emotion | value | User state |
| 1 | Pleasant | 5 | Extremely pleasant state |
| 2 | Pleasant | 4 | Very pleasant state |
| 3 | Pleasant | 3 | Moderately pleasant state |
| 4 | Pleasant | 2 | Slightly pleasant state |
| 5 | Pleasant | 1 | Barely pleasant state |
| . . . | . . . | . . . | . . . |

As described above, the action determination unit 236 determines the action content of the robot 100 in accordance with the state related to the emotion of the robot 100 determined in advance for each type of emotion of the robot 100 and for each intensity of the emotion, and the action of the user 10. In this embodiment, the utterance content of the robot 100 in a case in which an interaction with the user 10 is performed can be branched according to the state related to the emotion of the robot 100. That is, since the robot 100 can change the action of the robot according to the index number associated with the emotion of the robot, the user receives an impression that the robot has a mind, and is promoted to take an action such as talking to the robot.

Furthermore, the action determination unit 236 may generate the action content of the robot by adding a fixed sentence for asking a question about the action content of the robot corresponding to the action of the user and inputting the fixed sentence to the sentence generation model having the interaction function after adding not only the text indicating the action of the user, the emotion of the user, and the emotion of the robot but also the text indicating the content of the history data 222. As a result, the robot 100 can change the action of the robot according to the history data indicating the emotion and action of the user, and thus, the user receives an impression that the robot has personality, and is promoted to take an action such as talking to the robot. Furthermore, the history data may further include emotions and actions of the robot.

Furthermore, the emotion determination unit 232 may determine the emotion of the robot 100 based on the action content of the robot 100 generated by using the sentence generation model. Specifically, the emotion determination unit 232 inputs the action content of the robot 100 generated by using the sentence generation model to the pre-trained neural network, acquires the emotion value indicating each emotion indicated in the emotion map 400, integrates the acquired emotion value indicating each emotion and the current emotion value indicating each emotion of the robot 100, and updates the emotion of the robot 100. For example, the acquired emotion value indicating each emotion and the current emotion value indicating each emotion of the robot 100 are averaged and integrated. This neural network is pre-trained based on multiple pieces of training data that are combinations of texts representing the action contents of the robot 100 generated by using the sentence generation model and the emotion values representing the emotions shown in the emotion map 400.

For example, in a case in which, as an action content of the robot 100 generated by using the sentence generation model, an utterance content of the robot 100 “That was good. It was lucky.” is obtained, if a text indicating the utterance content is input into the neural network, the emotion of the robot 100 is updated such that a high value is obtained as the emotion value for the emotion “joyful” and the emotion value for the emotion “joyful” increases.

In the robot 100, a method is executed in which a sentence generation model such as generative AI and the emotion determination unit 232 are linked to each other, have an ego, and continue to grow with various parameters even while the user is not speaking.

The generative AI is a large-scale language model using a deep learning method. A technology is known in which, generative AI can also refer to external data, and for example, in ChatGPT plugins, various external data such as weather information and hotel reservation information is referred to through an interaction to output answers as accurately as possible. For example, when the generative AI is given a goal in natural language, the generative AI automatically generates source code in various programming languages. For example, when given a problematic source code, the generative AI performs debugging to find a problem, and can automatically generate an improved source code. In combination with the above, an autonomous agent that repeats code generation and debugging when given a goal in natural language until there is no problem in the source code has appeared. As such an autonomous agent, AutoGPT, babyAGI, JARVIS, E2B, and the like are known.

In the robot 100 according to the present embodiment, event data for training may be left in a database containing impressive memories by using a technique described in Patent Literature 2 (Japanese Patent No. 619992) in which the robot leaves event data for which the robot felt strong emotions for a long time and quickly forgets event data for which not much emotion was evoked towards the robot.

Further, the robot 100 may record the video data and the like of the user 10 acquired by the camera function and the like in the history data 222. The robot 100 may acquire video data and the like from the history data 222 as necessary and provide the video data and the like to the user 10. The robot 100 may generate video data having a larger information amount as the intensity of emotion is stronger and record the video data in the history data 222. For example, in a case in which information in a high-compression format such as skeleton data is recorded, the robot 100 may switch to recording of information in a low-compression format such as an HD moving image in response to the emotion value of excitement exceeding a threshold value. According to the robot 100, for example, it is possible to leave high-definition video data when the emotion of the robot 100 increases as a record.

When the robot 100 is not talking with the user 10, the robot 100 may automatically load the event data from the history data 222 in which the impressive event data is stored, and the emotion determination unit 232 may continue to update the emotion of the robot. When the robot 100 is not talking with the user 10 and the emotion of the robot 100 becomes an emotion encouraging learning, the robot 100 can create an emotion change event for changing the emotion of the user 10 to be good based on the impressive event data. As a result, autonomous learning (recollection of event data) at an appropriate timing according to the emotional state of the robot 100 can be realized, and autonomous learning appropriately reflecting the state of the emotion of the robot 100 can be realized.

The emotion encouraging learning is the emotion of “repentance” or “remorse” on the emotion map of Dr. Mitsuyoshi in a negative state, and the emotion of “desiring” on the emotion map in a positive state.

In the negative state, the robot 100 may treat “repentance” and “remorse” on the emotion map as emotions encouraging learning. In the negative state, the robot 100 may treat emotions adjacent to “repentance” and “remorse” as emotions encouraging learning, in addition to “repentance” and “remorse” on the emotion map. For example, the robot 100 treats at least one of “shame”, “stubbornness”, “self-destruction”, “self-precaution”, “regret”, or “despair” as an emotion encouraging learning, in addition to “repentance” and “remorse”. As a result, for example, when the robot 100 has a negative feeling such as “I do not want to have such a feeling again” or “I do not want to be reprimanded”, the robot can autonomously execute learning.

In a positive state, the robot 100 may treat “desiring” on the emotion map as an emotion encouraging learning. In a positive state, the robot 100 may treat an emotion adjacent to “desiring” as an emotion encouraging learning, in addition to “desiring”. For example, the robot 100 treats at least one of “joyful”, “euphoria”, “craving”, “expectation”, or “shame” as an emotion encouraging learning, in addition to “desire”. As a result, for example, when the robot 100 has a positive feeling such as “more desiring” or “want to know more”, autonomous learning can be executed.

The robot 100 may not execute autonomous learning when the robot 100 has an emotion other than the emotions encouraging learning as described above. As a result, for example, it is possible to prevent autonomous learning from being executed when the robot is extremely angry or blindly feeling love.

An emotion change event is, for example, to propose an action arising after an impressive event. An action after an impressive event is involved with an emotion label on the outermost side of the emotion map, and for example, the action of “tolerance” or “acceptance” that follow “love”.

In the autonomous learning executed when the robot 100 is not talking with the user 10, the emotion change event is created using the sentence generation model by combining the emotions, situations, actions, and the like of the people appearing in impressive memories and the robot itself.

Assuming that all emotion values are expressed by a six-stage evaluation of 0 to 5, a case in which event data “A friend was hit and looked displeased” is stored in the history data 222 as impressive event data is conceivable. Here, it is assumed that the friend refers to the user 10, the emotion of the user 10 is “antipathy”, and 5 has been input as the value indicating “antipathy”. Furthermore, it is assumed that the emotion of the robot 100 is “anxiety”, and 4 has been input as the value indicating “anxiety”.

The robot 100 can continue to grow with various parameters by performing an autonomous process while not talking with the user 10. Specifically, for example, as the uppermost event data arranged in descending order of emotion values, the event data “A friend was hit and looked displeased” is loaded from the history data 222. It is assumed that “anxiety” at intensity 4 is associated with the loaded event data as the emotion of the robot 100, and here, “antipathy” at intensity 5 is associated with the emotion of the user 10 who is a friend. If the current emotion value of the robot 100 is “relief” at intensity 3 before loading, the influence of “anxiety” at intensity 4 and “antipathy” at intensity of 5 is added after loading, and the emotion value of the robot 100 may change to “regret” meaning “frustrating”. At this time, since the emotion “regret” is an emotion encouraging learning, the robot 100 determines to recall the event data as the robot action and creates an emotion change event. At this time, the information input to the sentence generation model is a text representing the impressive event data, and in the present example, “a friend was hit and looked displeased”. Furthermore, in the emotion map, there is an emotion of “antipathy” on the innermost side, and an “attack” is predicted on the outermost side as an action corresponding to the emotion, and thus, in the present example, an emotion change event is created so as to prevent the friend from “attacking” someone.

For example, information of impressive event data can be used to solve the filling problem to automatically generate the following input text.

“The user was being hit. At that time, the user had extreme antipathy. The robot was very anxious. Please tell us 30 characters or less of the lines to say when the robot next meets the user. However, please make sure that it is not related to the time slot of meeting. Also, please avoid direct expressions. Three candidates will be listed.

### <Expected Format>

Candidate 1: (words that the robot should speak to the user)
Candidate 2: (words that the robot should speak to the user)
Candidate 3: (words that the robot should speak to the user)”

At this time, the output of the sentence generation model is, for example, as follows.
“Candidate 1: OK?I was worried about what happened yesterday.
Candidate 2: I was worried about what happened yesterday. What should I do?
Candidate 3: I was worried. Could you say something?”

Furthermore, the robot 100 may automatically generate the following input text for the information obtained by creating an emotion change event.

In a case in which “the user was being hit”, how will the user feel when the next message is spoken to the user? It is assumed that emotions of the user are in the form of “joy A, anger B, sorrow C, and pleasure D”, and A to D are integers of six-stage evaluation from 0 to 5.
Candidate 1: OK?I was worried about what happened yesterday.
Candidate 2: I was worried about what happened yesterday. What should I do?
Candidate 3: I was worried. Could you say something?”

At this time, the output of the sentence generation model is, for example, as follows.

“The emotions of the user may be as follows;
Candidate 1: Joy 3, anger 1, sorrow 2, pleasure 2
Candidate 2: Joy 2, anger 1, sorrow 3, pleasure 2; and
Candidate 3: Joy 2, anger 1, sorrow 3, pleasure 3”

In this manner, the robot 100 may execute the process of thinking after creating an emotion change event.

Finally, the robot 100 may create an emotion change event by using the candidate 1 that is most likely to make the user joyful among the multiple candidates, store the emotion change event in the action plan data 224, and prepare for the next meeting with the user 10.

As described above, even when not having a conversation with a family member or a friend, the emotion value of the robot 100 is continuously determined using the information of the history data 222 in which the impressive event data is stored, and when the robot has the emotion encouraging learning, the robot 100 executes autonomous learning when not having a conversation with the user 10 according to the emotion of the robot 100, and continues to update the history data 222 and the action plan data 224.

Although the above is an example using emotion values, in the emotion map, the emotion can be generated from the amount of hormone secreted and the event type, and therefore, the values associated with the impressive event data may be the type of hormone, the amount of hormone secreted, and the type of event.

Hereinafter, specific examples will be described.

For example, even when not talking with the user, the robot 100 investigates information regarding a topic or hobby of interest to the user.

For example, even when not talking with the user, the robot 100 investigates information regarding the birthday or anniversaries of the user and considers a congratulatory message.

For example, even when not talking with the user, the robot 100 investigates reviews of a place that the user wants to go to, food, or products.

For example, even when not talking with the user, the robot 100 investigates weather information and provides advice suitable for the user's schedule or plan.

For example, even when not talking with the user, the robot 100 investigates information on local events and festivals and proposes the information to the user.

For example, even when not talking with the user, the robot 100 investigates game results or news of a sport of interest of the user and provides a topic.

For example, even when not talking with the user, the robot 100 investigates and introduces information of the user's favorite music or artists.

For example, even when not talking with the user, the robot 100 investigates information regarding social problems or news that the user is interested in and provides opinions.

For example, even when not talking with the user, the robot 100 investigates information regarding the user's hometown or places of origin and provides a topic.

For example, even when not talking with the user, the robot 100 investigates information of the user's work or school and provides advice.

Even when not talking with the user, the robot 100 investigates and introduces information of books, comics, movies, and drama that the user is interested in.

For example, even when not talking with the user, the robot 100 investigates information regarding health of the user and provides advice.

For example, even when not talking with the user, the robot 100 investigates information regarding travel planning of the user and provides advice.

For example, even when not talking with the user, the robot 100 investigates information regarding repair or maintenance of the house or car of the user and provides advice.

For example, even when not talking with the user, the robot 100 investigates information on beauty and fashion that the user is interested in and provides advice.

For example, even when not talking with the user, the robot 100 investigates information of the pet of the user and provides advice.

For example, even when not talking with the user, the robot 100 investigates and proposes information of contests and events related to the user's hobby or work.

For example, even when not talking with the user, the robot 100 investigates information of the user's favorite restaurant or eateries and proposes the information.

For example, even when not talking with the user, the robot 100 collects information and provides advice regarding important decisions related to the user's life.

For example, even when not talking with the user, the robot 100 investigates information regarding a person the user is worried about and provides advice.

### Second Embodiment

In a second embodiment, the robot 100 is applied to a control device mounted on a stuffed toy or connected wirelessly or by wire to a control target device (speaker or camera) mounted on a stuffed toy. Note that parts having the same configurations as those of the first embodiment are denoted by the same reference numerals, and description thereof is omitted.

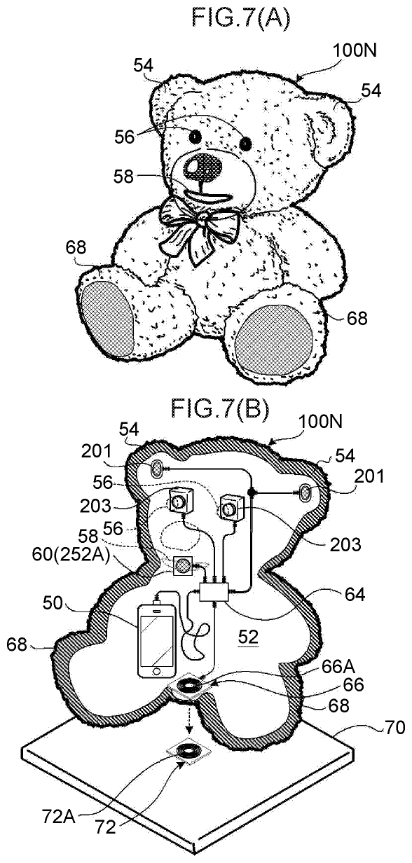

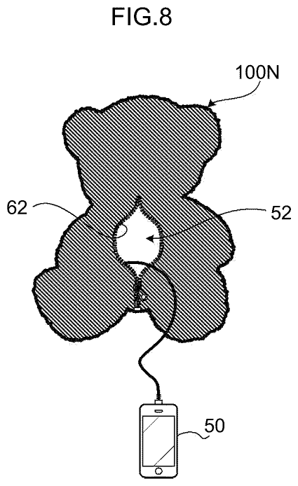

Specifically, the second embodiment is configured as follows. For example, the robot 100 is applied to a co-dweller (specifically, a stuffed toy 100N illustrated in FIGS. 7 and 8) that has conversations with the user 10 based on information regarding daily life while spending daily life with the user 10 or provides information aligned with a hobby and preference of the user 10. In the second embodiment, an example in which the control part of the robot 100 is applied to a smartphone 50 will be described.

The stuffed toy 100N having a function as an input/output device of the robot 100 has the smartphone 50 that is detachable therefrom functioning as a control part of the robot 100, and the input/output device and the accommodated smartphone 50 are connected inside the stuffed toy 100N.

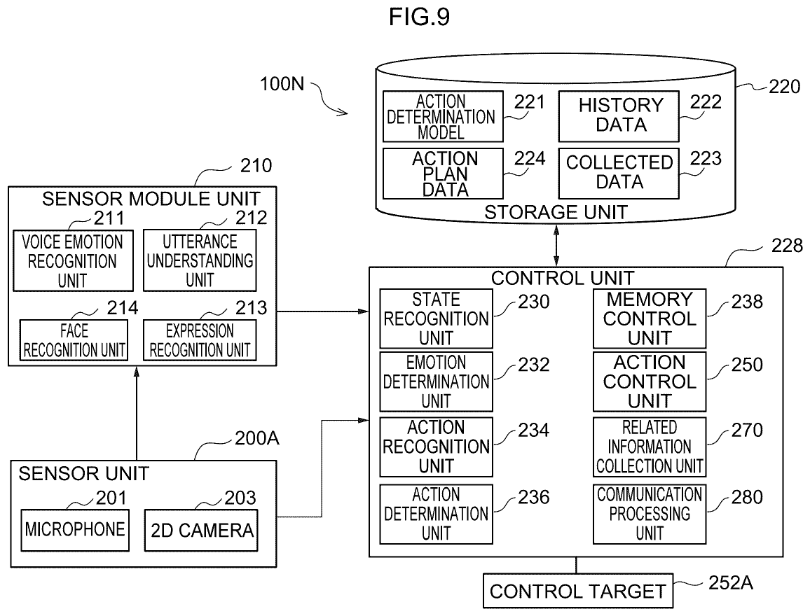

As illustrated in FIG. 7(A), the stuffed toy 100N has a shape of a bear covered with a soft cloth fabric in the present embodiment (and other embodiments), and a sensor unit 200A and a control target 252A are arranged as input/output devices in a space portion 52 formed inside the stuffed toy (see FIG. 9). The sensor unit 200A includes a microphone 201 and a 2D camera 203. Specifically, as illustrated in FIG. 7(B), in the space portion 52, the microphone 201 of the sensor unit 200 is disposed in a portion corresponding to ears 54, the 2D camera 203 of the sensor unit 200 is disposed in a portion corresponding to the eyes 56, and the speaker 60 constituting a part of the control target 252A is disposed in a portion corresponding to the mouth 58. Note that the microphone 201 and the speaker 60 are not necessarily separated from each other, and may be an integrated unit. In the case of the unit, it is preferable to arrange the unit at a position where the utterance can be heard naturally, such as the position of the nose of the stuffed toy 100N. Note that, although the case in which the stuffed toy 100N has an animal shape has been described as an example, the present invention is not limited thereto. The stuffed toy 100N may have the shape of a specific character.

FIG. 9 schematically illustrates a functional configuration of the stuffed toy 100N. The stuffed toy 100N includes the sensor unit 200A, a sensor module unit 210, a storage unit 220, a control unit 228, and a control target 252A.

The smartphone 50 housed in the stuffed toy 100N of the present embodiment performs processing similar to that of the robot 100 of the first embodiment. That is, the smartphone 50 has the function as the sensor module unit 210, the function as the storage unit 220, and the function as the control unit 228 illustrated in FIG. 9.

As illustrated in FIG. 8, a fastener 62 is attached to a part (for example, the back portion) of the stuffed toy 100N, and the outside and the space portion 52 communicate with each other by opening the fastener 62.

Here, the smartphone 50 is accommodated in the space portion 52 from the outside and is connected to each input/output device via a USB hub 64 (see FIG. 7(B)) in a USB manner, so that it is possible to have functions equivalent to those of the robot 100 of the first embodiment.

Further, a contactless power receiving plate 66 is connected to a USB hub 64. A power receiving coil 66A is incorporated in the power receiving plate 66. The power receiving plate 66 is an example of a wireless power receiving unit that receives wireless power supply.

The power receiving plate 66 is disposed near root portions 68 of both feet of the stuffed toy 100N, and is positioned closest to a mounting base 70 when the stuffed toy 100N is placed on the mounting base 70. The mounting base 70 is an example of an external wireless power transmission unit.

The stuffed toy 100N placed on the mounting base 70 can be appreciated as an ornament in a natural state.

In addition, these root portions are formed to be thinner than the surface thickness of the stuffed toy 100N in other parts, and are held in a state closer to the mounting base 70.

The mounting base 70 includes a charging pad 72. A power transmitting coil 72A is incorporated in the charging pad 72, and when the power transmitting coil 72A transmits a signal to search for the power receiving coil 66A of the power receiving plate 66 and the power receiving coil 66A is found, a current flows through the power transmitting coil 72A to generate a magnetic field, and the power receiving coil 66A reacts to the magnetic field to start electromagnetic induction. As a result, current flows through the power receiving coil 66A, and power is stored in a battery (not shown) of the smartphone 50 via the USB hub 64.

That is, since the smartphone 50 is automatically charged by placing the stuffed toy 100N as an ornament on the mounting base 70, it is not necessary to take out the smartphone 50 from the space portion 52 of the stuffed toy 100N for charging.

Note that, in the second embodiment, the smartphone 50 is accommodated in the space portion 52 of the stuffed toy 100N and connected by wire (USB connection), but the invention is not limited thereto. For example, a control device having a wireless function (for example, “Bluetooth (registered trademark)”) may be accommodated in the space portion 52 of the stuffed toy 100N, and the control device may be connected to the USB hub 64. In this case, the smartphone 50 and the control device wirelessly communicate with each other without inserting the smartphone 50 into the space portion 52, and the external smartphone 50 is connected to each input/output device via the control device, so that it is possible to provide functions equivalent to those of the robot 100 of the first embodiment. Furthermore, the control device which is accommodated in the space portion 52 of the stuffed toy 100N and the external smartphone 50 may be connected by wire.

Furthermore, although the stuffed bear 100N has been exemplified in the second embodiment, the shape may be another animal, a doll, or a shape of a specific character. Further, the clothes may be changeable. Furthermore, the material of the skin is not limited to the cloth fabric, and may be other materials such as soft vinyl, but is preferably a soft material.

Furthermore, a monitor may be attached to the skin of the stuffed toy 100N, and the control target 252 that provides information to the user 10 through vision may be added. For example, the eyes 56 may be used as a monitor to express joy, anger, sorrow, and pleasure using images projected on the eyes, or a window through which the monitor of the built-in smartphone 50 is transmitted may be provided in the abdomen. Furthermore, the eyes 56 may be used as a projector to express joy, anger, sorrow, and pleasure by using an image projected on a wall surface.

According to the second embodiment, the existing smartphone 50 is placed in the stuffed toy 100N, and the camera 203, the microphone 201, the speaker 60, and the like are extended from the place to appropriate positions via the USB connection.

Further, for wireless charging, the smartphone 50 and the power receiving plate 66 are connected via USB, and the power receiving plate 66 is disposed so as to be as outside as possible when viewed from the inside of the stuffed toy 100N.

In order to use wireless charging of the smartphone 50, it is necessary to arrange the smart phone 50 as outside as possible when viewed from the inside of the stuffed toy 100N, and the stuffed toy 100N is rough when touched from the outside.

Therefore, the smartphone 50 is disposed at the center of the stuffed toy 100N as much as possible, and the wireless charging function (power receiving plate 66) is disposed outside as viewed from the inside of the stuffed toy 100N as much as possible. The camera 203, the microphone 201, the speaker 60, and the smartphone 50 receive wireless power supply via the power receiving plate 66.

Note that other configurations and effects of the stuffed toy 100N of the second embodiment are similar to those of the robot 100 of the first embodiment, and thus the description thereof will be omitted.

Further, a part of the stuffed toy 100N (for example, the sensor module unit 210, the storage unit 220, and the control unit 228) may be provided outside the stuffed toy 100N (for example, the server), and the stuffed toy 100N may function as each part of the stuffed toy 100N by communicating with the outside.

### Third Embodiment

In the first embodiment, the case in which the action control system is applied to the robot 100 has been exemplified, but in the third embodiment, the robot 100 is used as an agent for interacting with a user, and the action control system is applied to an agent system. Note that parts having the same configurations as those of the first and second embodiments are denoted by the same reference numerals, and description thereof is omitted.

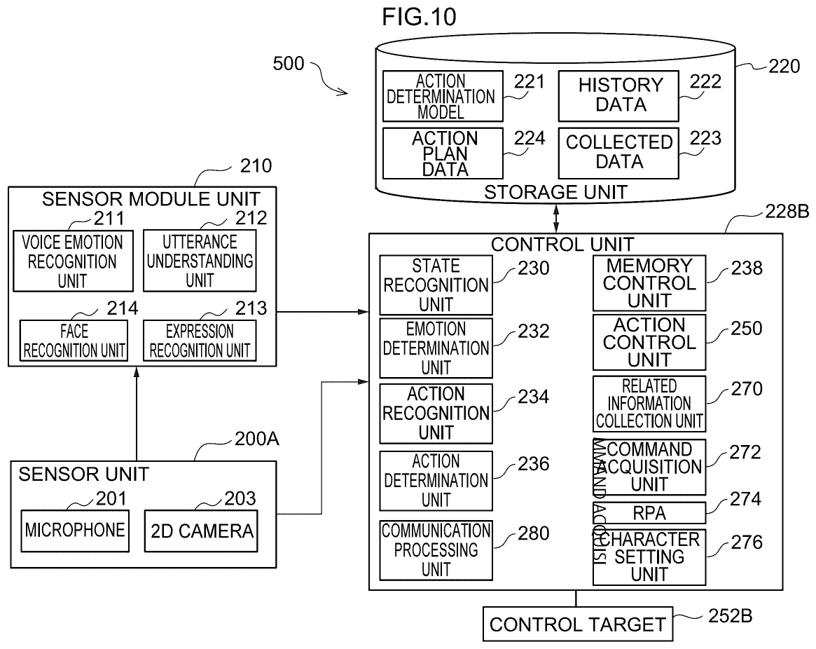

FIG. 10 is a functional block diagram of an agent system 500 configured using some or all of the functions of the action control system.

The agent system 500 is a computer system that performs a series of actions according to the intention of the user 10 through an interaction performed with the user 10. The interaction with the user 10 can be performed by voice or text.

The agent system 500 includes a sensor unit 200A, a sensor module unit 210, a storage unit 220, a control unit 228B, and a control target 252B.

The agent system 500 can be mounted on, for example, a robot, a doll, a stuffed toy, a wearable terminal (pendants, smartwatches, smart glasses), a smartphone, a smart speaker, earphones, a personal computer, or the like. Furthermore, the agent system 500 may be implemented in a web server and used via a web browser operating on a communication terminal such as a smartphone carried by the user.

The agent system 500 serves as, for example, a butler, a secretary, a teacher, a partner, a friend, a lover, or a teacher acting for the user 10. The agent system 500 not only interacts with the user 10 but also provides advice, guides to a destination, gives recommendations according to user's preference, or the like. In addition, the agent system 500 performs reservation, order, payment, or the like to a service provider.

The emotion determination unit 232 determines an emotion of the user 10 and an emotion of the agent itself, similarly in the first embodiment. The action determination unit 236 determines an action of the robot 100 in consideration of emotions of the user 10 and the agent. In other words, the agent system 500 understands the emotion of the user 10 and reads the air to realize heartfelt support, assistance, advice, and service provision. Furthermore, the agent system 500 comforts, encourages, and energizes the user by listening to concerns of the user 10. Furthermore, the agent system 500 plays with the user 10 and draws a picture diary to remind the user of the past. The agent system 500 performs an action that increases the sense of happiness of the user 10. Here, the agent refers to an agent that operates on software.

The control unit 228B includes a state recognition unit 230, an emotion determination unit 232, an action recognition unit 234, an action determination unit 236, a memory control unit 238, an action control unit 250, a related information collection unit 270, a command acquisition unit 272, Robotic Process Automation (RPA) 274, a character setting unit 276, and a communication processing unit 280.

As in the first embodiment, the action determination unit 236 determines an utterance content of the agent for interacting with the user 10 as an action of the agent. The action control unit 250 outputs the utterance content of the agent using at least one of voice or text through a speaker or a display that serves as the control target 252B.

The character setting unit 276 sets a character of the agent when the agent system 500 interacts with the user 10 based on designation by the user 10. In other words, the utterance content output from the action determination unit 236 is output through the agent having the set character. As the character, for example, a real famous figure or a famous person such as an actor, an entertainer, an idol, or a sport player can be set. Furthermore, it is also possible to set a fictitious character appearing in a cartoon, a movie, or an animation. In a case in which the character of the agent is known, since the voice, the wording, the tone, and the personality of the character are known, the character setting unit 276 can automatically set prompts only by the user 10 designating his/her favorite character. The voice, the wording, the tone of voice, and the personality of the set character are reflected in the interaction with the user 10. In other words, the action control unit 250 synthesizes a voice corresponding to the character set by the character setting unit 276, and outputs the utterance content of the agent in the synthesized voice. As a result, the user 10 can feel as if he/she is interacting with his/her favorite character (for example, a favorite actor).

In a case in which the agent system 500 is mounted on a device having a display such as a smartphone, for example, an icon, a still image, or a moving image of the agent having a character set by the character setting unit 276 may be displayed on the display. The image of the agent is generated using, for example, an image synthesis technology such as 3D rendering. In the agent system 500, an interaction with the user 10 may be performed while the image of the agent performs a gesture according to the emotion of the user 10, the emotion of the agent, and the utterance content of the agent. Note that the agent system 500 may output only voice without outputting an image when interacting with the user 10.

As in the first embodiment, the emotion determination unit 232 determines an emotion value indicating the emotion of the user 10 and an emotion value of the agent itself. In the present embodiment, the emotion value of the agent is determined instead of the emotion value of the robot 100. The emotion value of the agent itself is reflected in the emotion of the set character. When the agent system 500 interacts with the user 10, not only the emotion of the user 10 but also the emotion of the agent is reflected in the interaction. In other words, the action control unit 250 outputs the utterance content in a mode according to the emotion determined by the emotion determination unit 232.

Furthermore, the emotion of the agent is also reflected in a case in which the agent system 500 performs an action toward the user 10. For example, in a case in which the user 10 requests the agent system 500 to take a photo, whether or not the agent system 500 takes a photo in response to the request from the user is determined according to the degree of “sadness” felt by the agent. In a case in which the character has a positive emotion, the character performs a favorable interaction or action with respect to the user 10, and in a case in which the character has a negative emotion, the character performs a defiant interaction or action with respect to the user 10.

The history data 222 stores a history of the interactions performed between the user 10 and the agent system 500 as event data. The storage unit 220 may be realized by an external cloud storage. In a case of interacting with the user 10 or performing an action toward the user 10, the agent system 500 decides the interaction content or the action content in consideration of the content of the interaction history stored in the history data 222. For example, the agent system 500 grasps hobbies and preferences of the user 10 based on the interaction history stored in the history data 222. The agent system 500 generates an interaction content matching the hobbies and preferences of the user 10 and provides a recommendation. The action determination unit 236 determines the utterance content of the agent based on the interaction history stored in the history data 222. In the history data 222, personal information such as the name, address, telephone number, and credit card number of the user 10 acquired through interactions with the user 10 is stored. Here, an agent may spontaneously make an utterance of inquiry about whether or not to register personal information with the user 10, such as “Do you want me to register your credit card number?”, and the personal information may be stored in the history data 222 according to the answer of the user 10.

As described in the first embodiment, the action determination unit 236 generates the utterance content based on the sentence generated using the sentence generation model.

Specifically, the action determination unit 236 inputs the text or voice input by the user 10 and the emotions of both the user 10 and the character determined by the emotion determination unit 232, and the conversation history stored in the history data 222 to the sentence generation model to generate the utterance content of the agent. At this time, the action determination unit 236 may further input the character's personality set by the character setting unit 276 to the sentence generation model to generate the utterance content of the agent. In the agent system 500, the sentence generation model is not located on the front-end side serving as a touch point for the user 10, but is used solely as a tool of the agent system 500.

The command acquisition unit 272 uses the output of the utterance understanding unit 212 to acquire a command of the agent from a voice or a text uttered from the user 10 through an interaction with the user 10. The command includes, for example, contents of actions to be executed by the agent system 500, such as information search, store reservation, ticket arrangement, purchase of products/services, payment, route guidance to a destination, and recommendation provision.

The RPA 274 performs an action according to the command acquired by the command acquisition unit 272. For example, the RPA 274 performs actions related to use of the service provider, such as information search, store reservation, ticket arrangement, purchase of products/services, and payment.

The RPA 274 reads the personal information of the user 10 necessary for executing the action related to the use of the service provider from the history data 222 and uses the personal information. For example, in a case of purchasing a product in response to a request from the user 10, the agent system 500 reads and uses personal information such as the name, address, telephone number, and credit card number of the user 10 stored in the history data 222. Requesting the user 10 to input personal information in the initial setting is unkind, giving discomfort to the user. In the agent system 500 according to the present embodiment, instead of requesting the user 10 to input personal information in the initial setting, the personal information acquired through interactions with the user 10 is stored, and used by reading if necessary. As a result, it is possible to avoid making the user feel any discomfort, and convenience of the user is improved.

The agent system 500 executes an interactive process by, for example, following steps 1 to 6.

(Step 1) The agent system 500 sets a character of the agent. Specifically, the character setting portion 276 sets a character of the agent when the agent system 500 interacts with the user 10 based on designation by the user 10.

(Step 2) The agent system 500 acquires the state of the user 10 including the voice or text input from the user 10, the emotion value of the user 10, the emotion value of the agent, and the history data 222. Specifically, the process similar to steps S100 to S103 is performed to acquire the state of the user 10 including the voice or text input from the user 10, the emotion value of the user 10, the emotion value of the agent, and the history data 222.

(Step 3) The agent system 500 determines the utterance content of the agent.

Specifically, the action determination unit 236 inputs the text or voice input by the user 10, the emotions of both the user 10, the character determined by the emotion determination unit 232, and the conversation history stored in the history data 222 to the sentence generation model to generate the utterance content of the agent.

For example, the utterance content of the agent is acquired by adding a fixed sentence “At this time, what would you answer as an agent?” to the text or voice input by the user 10, the text indicating the emotions of both the user 10 and the character specified by the emotion determination unit 232 and the conversation history stored in the history data 222, and inputting the fixed sentence to the sentence generation model.

As an example, in a case in which the text or voice input to the user 10 is “I want you to reserve a close nice Chinese restaurant for 7 this evening”, an utterance content of the agent such as “Understood.” and “These are recommendable restaurants. 1. AAAA. 2. BBBB. 3. CCCC. 4. DDDD” is obtained.

Furthermore, in a case in which the text or voice input to the user 10 is “No. 4 DDDD sounds good”, an utterance content of the agent such as “Certainly. I will make a reservation. How many seats?” is obtained.

(Step 4) The agent system 500 outputs the utterance content of the agent.

Specifically, the action control unit 250 synthesizes a voice corresponding to the character set by the character setting unit 276, and outputs the utterance content of the agent in the synthesized voice.

(Step 5) The agent system 500 determines whether or not it is a timing to execute the command of the agent.

Specifically, the action determination unit 236 determines whether or not it is a timing to execute the command of the agent based on the output of the sentence generation model. For example, in a case in which the output of the sentence generation model includes that the agent should execute the command, it is determined that it is the timing to execute the command of the agent, and the process proceeds to step 6. On the other hand, in a case in which it is determined that it is not the timing to execute the command of the agent, the process returns to step 2 described above.

(Step 6) The agent system 500 executes the command of the agent.

Specifically, the command acquisition unit 272 acquires the command of the agent from the voice or text uttered from the user 10 through the interaction with the user 10. Then, the RPA 274 performs an action corresponding to the command acquired by the command acquisition unit 272. For example, in a case in which the command is “information search”, information search is performed by using a search site using a search query obtained through an interaction with the user 10 and an application programming interface (API). The action determination unit 236 inputs the search result to the sentence generation model to generate the utterance content of the agent. The action control unit 250 synthesizes a voice corresponding to the character set by the character setting unit 276, and outputs the utterance content of the agent by using the synthesized voice.

Furthermore, in a case in which the command is “store reservation”, the reservation is made by making a phone call to the store to be reserved using the reservation information obtained through the interaction with the user 10, information of the store to be reserved, and the API using the phone software. At this time, the action determination unit 236 acquires the utterance content of the agent with respect to the voice input from the partner using the sentence generation model having the interaction function. Then, the action determination unit 236 inputs the result of the store reservation (whether or not the reservation is successful) to the sentence generation model to generate the utterance content of the agent. The action control unit 250 synthesizes a voice corresponding to the character set by the character setting unit 276, and outputs the utterance content of the agent by using the synthesized voice.

Then, the process returns to step 2 described above.

In step 6, the result of the action (for example, store reservation) executed by the agent is also stored in the history data 222. The result of the action executed by the agent stored in the history data 222 is used by the agent system 500 to grasp hobbies or preferences of the user 10. For example, in a case in which the same store has been reserved multiple times, it is recognized that the user 10 likes the store, or the reservation details such as the time slot for reservation, or details of the course, or the fee are used as a criterion for choosing the store for reservation of the next time.

In this manner, the agent system 500 can execute the interaction processing and perform an action related to use of the service provider if necessary.

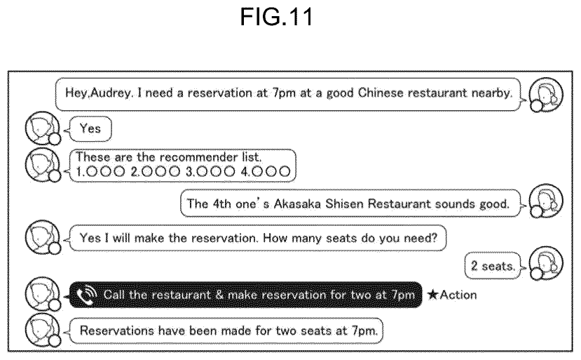

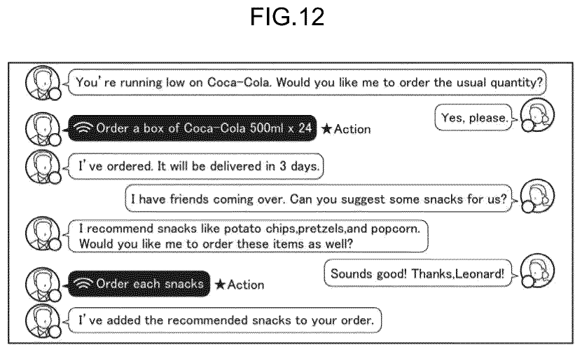

FIG. 11 and FIG. 12 illustrate an example of an operation of the agent system 500. FIG. 11 illustrates a mode in which the agent system 500 makes a restaurant reservation through an interaction with the user 10. In FIG. 11, the utterance contents of the agent are shown on the left side, and the utterance contents of the user 10 are shown on the right side. The agent system 500 can ascertain preferences of the user 10 based on an interaction history with respect to the user 10, provide a list of restaurant recommendations that match the preferences of the user 10, and perform a reservation for a selected restaurant.

Meanwhile, FIG. 12 illustrates a mode in which the agent system 500 accesses an e-commerce site through the interaction with the user 10 to purchase the product. In FIG. 12, the utterance contents of the agent are shown on the left side, and the utterance contents of the user 10 are shown on the right side. The agent system 500 can estimate the remaining amount of the beverage stocked by the user based on the interaction history with respect to the user 10, and can propose purchase of the beverage to the user 10 and execute purchase. Furthermore, the agent system 500 can grasp the preferences of the user based on the past interaction history with respect to the user 10, and recommend a snack that the user likes. In this manner, the agent system 500 supports daily life of the user 10 by performing various actions such as restaurant reservation or product purchase and payment while communicating with the user 10 as an agent such as a butler.

Note that other configurations and operations of the agent system 500 of the third embodiment are similar to those of the robot 100 of the first embodiment, and thus description thereof is omitted.

Furthermore, a part of the agent system 500 (for example, the sensor module unit 210, the storage unit 220, and the control unit 228B) may be provided outside a communication terminal such as a smartphone carried by the user (for example, on a server), and the communication terminal may function as each unit of the agent system 500 by communicating with the outside.

### Fourth Embodiment

In a fourth embodiment, the agent system is applied to smart glasses. Note that parts having the same configurations as those of the first to third embodiments are denoted by the same reference numerals, and description thereof is omitted.

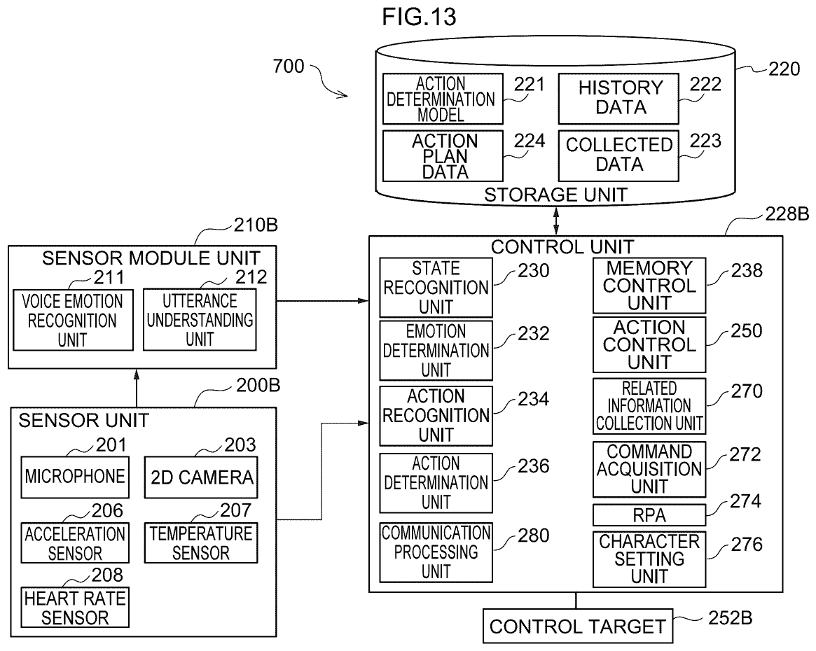

FIG. 13 is a functional block diagram of an agent system 700 configured using some or all of the functions of the action control system. The agent system 700 includes a sensor unit 200B, a sensor module unit 210B, a storage unit 220, a control unit 228B, and a control target 252B. The control unit 228B includes a state recognition unit 230, an emotion determination unit 232, an action recognition unit 234, an action determination unit 236, a memory control unit 238, an action control unit 250, a related information collection unit 270, a command acquisition unit 272, an RPA 274, a character setting unit 276, and a communication processing unit 280.

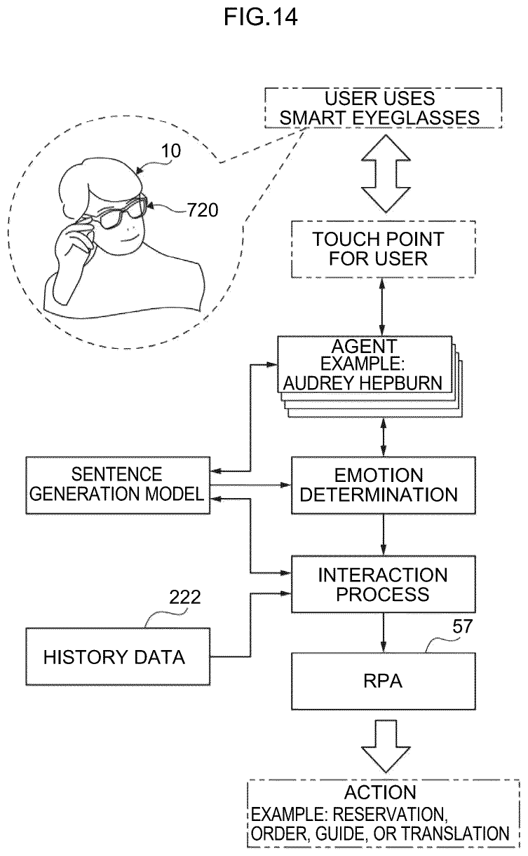

As illustrated in FIG. 14, the smart glasses 720 are a glasses-type smart device, and are worn by the user 10 similarly to general glasses. The smart glasses 720 are an example of electronic equipment and a wearable terminal.

The smart glasses 720 include the agent system 700. The display included in the control target 252B displays various types of information to the user 10. The display is, for example, a liquid crystal display. The display is provided, for example, in a lens portion of the smart glasses 720, and the display content can be visually recognized by the user 10. The speaker included in the control target 252B outputs a voice indicating various types of information to the user 10. The smart glasses 720 include a touch panel (not illustrated), and the touch panel receives inputs from the user 10.

An acceleration sensor 206, a temperature sensor 207, and a heart rate sensor 208 of the sensor unit 200B detect states of the user 10. Note that these sensors are merely examples, and it is a matter of course that other sensors may be mounted to detect states of the user 10.

A microphone 201 acquires voices uttered by the user 10 or environmental sounds around the smart glasses 720. A 2D camera 203 can image the surroundings of the smart glasses 720. The 2D camera 203 is, for example, a CCD camera.

The sensor module unit 210B includes a voice emotion recognition unit 211 and an utterance understanding unit 212. The communication processing unit 280 of the control unit 228B controls communication between the smart glasses 720 and the outside.

FIG. 14 is a diagram illustrating an example of a usage mode of the agent system 700 on the smart glasses 720. The smart glasses 720 realize provision of various services to the user 10 using the agent system 700. For example, when the user 10 operates the smart glasses 720 (for example, sound input to a microphone, or tapping the touch panel with a finger.), the smart glasses 720 start using the agent system 700. Here, using the agent system 700 includes modes in which the smart glasses 720 have the agent system 700 and use the agent system 700, and a part (for example, the sensor module unit 210B, the storage unit 220, and the control unit 228B) of the agent system 700 is provided outside the smart glasses 720 (for example, a server) and the smart glasses 720 communicate with the outside to use the agent system 700.

When the user 10 operates the smart glasses 720, a touch point is generated between the agent system 700 and the user 10. That is, provision of services by the agent system 700 is started. As described in the third embodiment, in the agent system 700, a character of the agent is set by the character setting unit 276.

The emotion determination unit 232 determines an emotion value indicating the emotion of the user 10 and an emotion value of the agent itself. Here, the emotion value indicating the emotion of the user 10 is estimated from various sensors included in the sensor unit 200B mounted on the smart glasses 720. For example, in a case in which a heart rate of the user 10 detected by the heart rate sensor 208 is increased, the emotion values for “anxiety” and “fear” are estimated to be high.

Furthermore, as a result of measuring the body temperature of the user by using the temperature sensor 207, for example, in a case in which the body temperature exceeds the average body temperature, the emotion value for “suffering” or “hardship” is estimated to be high. Furthermore, for example, in a case in which the acceleration sensor 206 detects that the user 10 is playing some kind of sport, the emotion value for “pleasant” is estimated to be large.

Furthermore, for example, the emotion value of the user 10 may be estimated from the voice or utterance content of the user 10 acquired by the microphone 201 mounted on the smart glasses 720. For example, in a case in which the user 10 is raising his/her voice, the emotion value for “anger” is estimated to be high.

In a case in which the emotion value estimated by the emotion determination unit 232 is higher than a predetermined value, the agent system 700 causes the smart glasses 720 to acquire information regarding the surrounding situation. Specifically, for example, the 2D camera 203 is caused to capture an image or a moving image representing a situation around the user 10 (for example, a person or an object within the surrounding area). Further, the microphone 201 is caused to record ambient environmental sound. Other examples of the information regarding the surrounding situation include information indicating date, time, positional information, weather, and the like. The information regarding the surrounding situation is stored in the history data 222 together with the emotion value. The history data 222 may be realized by an external cloud storage. As described above, the surrounding situation obtained by the smart glasses 720 is stored in the history data 222 as a so-called life log in a state of being associated with the emotion value of the user 10 at that time.

In the agent system 700, the information indicating the surrounding situation is stored in the history data 222 in association with the emotion value. As a result, the agent system 700 ascertains personal information such as hobbies, preferences, or personality of the user 10. For example, in a case in which an image representing a state of baseball game watching is associated with an emotion value for “joy” or “pleasant”, the hobby of the user 10 is baseball game watching, and the agent system 700 ascertains his/her favorite team or player from the information stored in the history data 222.

Then, in a case of interacting with the user 10 or performing an action toward the user 10, the agent system 700 determines the interaction content or the action content in consideration of the details of the surrounding situations stored in the history data 222. Note that, as a matter of course, the interaction content or the action content may be determined in consideration of the interaction history stored in the history data 222 as described above in addition to the surrounding situations.

As described above, the action determination unit 236 generates the utterance content based on the sentence generated by the sentence generation model. Specifically, the action determination unit 236 inputs the text or voice input by the user 10, the emotions of both the user 10 and the agent determined by the emotion determination unit 232, the conversation history stored in the history data 222, the personality of the agent, and the like to the sentence generation model to generate the utterance content of the agent. Furthermore, the action determination unit 236 inputs the surrounding situations stored in the history data 222 to the sentence generation model to generate the utterance content of the agent.

The generated utterance content is output in voice from a speaker mounted on the smart glasses 720 to the user 10, for example. In this case, a synthesized voice corresponding to the character of the agent is used as the voice. The action control unit 250 generates a synthesized voice by reproducing the voice quality of the character of the agent or generates a synthesized voice according to the emotion of the character (for example, in the case of the emotion “anger”, a voice in a strong tone). Furthermore, the utterance content may be displayed on the display instead of a voice output or together with a voice output.

The RPA 274 executes an operation according to a command (for example, a command of the agent acquired from a voice or text uttered by the user 10 through interactions with the user 10.). The RPA 274 performs actions related to use of service providers, such as information search, store reservation, ticket arrangement, purchase of products/services, payment, route guidance, and translation.

Furthermore, as another example, the RPA 274 executes an operation of transmitting a content input by voice of the user 10 (for example, a child) through interactions with the agent to the other party (for example, the parent). Examples of the transmission means include message application software, chat application software, mail application software, and the like.

In a case in which the operation by the RPA 274 is executed, for example, a voice indicating that the execution of the operation has been finished is output from a speaker mounted on the smart glasses 720. For example, a voice such as “Reservation for the store has been completed” is output to the user 10. Furthermore, for example, in a case in which reservation of the store is full, a voice indicating “Reservation could not be made. What would you like to do?” is output to the user 10.

Note that the smart glasses 720 may function as each unit of the agent system 700 when some units of the agent system 700 (for example, the sensor module unit 210B, the storage unit 220, and the control unit 228B) are provided outside the smart glasses 720 (for example, a server), and the smart glasses communicate with the outside.

As described above, with the smart glasses 720, various services are provided to the user 10 by using the agent system 700. In addition, since the smart glasses 720 are worn by the user 10, the agent system 700 can be used in various scenes such as at home, at work, and at a place outside the house.

In addition, since the smart glasses 720 are worn by the user 10, the smart glasses are suitable for collecting so-called life logs of the user 10. Specifically, an emotion value of the user 10 is estimated based on detection results by various sensors or the like mounted on the smart glasses 720 or recording results of the 2D camera 203 or the like. Therefore, emotion values of the user 10 can be collected in various scenes, and the agent system 700 can provide a service or utterance content suitable for the emotions of the user 10.

Furthermore, in the smart glasses 720, situations around the user 10 can be obtained by the 2D camera 203, the microphone 201, and the like. Then, these surrounding situations and the emotion values of the user 10 are associated with each other. As a result, it is possible to estimate what kind of emotion the user 10 has in what kind of situation. As a result, the accuracy in the agent system 700 to ascertain the hobbies/preferences of the user 10 can be improved. Then, in the agent system 700, the hobbies/preferences of the user 10 are accurately ascertained, and thereby the agent system 700 can provide a service or an utterance content suitable for the hobbies/preferences of the user 10.

Furthermore, the agent system 700 can also be applied to other wearable terminals (electronic equipment that can be worn on the body of the user 10, such as a pendant, a smart watch, an earring, a bracelet, or a hairband.). In a case in which the agent system 700 is applied to a smart pendant, a speaker as the control target 252B outputs a voice indicating various types of information to the user 10. The speaker is, for example, a speaker capable of outputting a voice having directivity. The speaker is set to have directivity toward the ears of the user 10.

As a result, the voice is prevented from reaching a person other than the user 10. The microphone 201 acquires a voice uttered by the user 10 or an environmental sound around the smart pendant. The smart pendant is worn in such a way that it hangs around the neck of the user 10. Thus, the smart pendant is located relatively close to the mouth of the user 10 while being worn. This facilitates acquisition of voices uttered by the user 10.

### Fifth Embodiment

In a fifth embodiment, the robot 100 is applied as an agent for interacting with a user through an avatar. That is, the action control system is applied to an agent system configured using a headset-type terminal. Note that parts having the same configurations as those of the first and second embodiments are denoted by the same reference numerals, and description thereof is omitted.

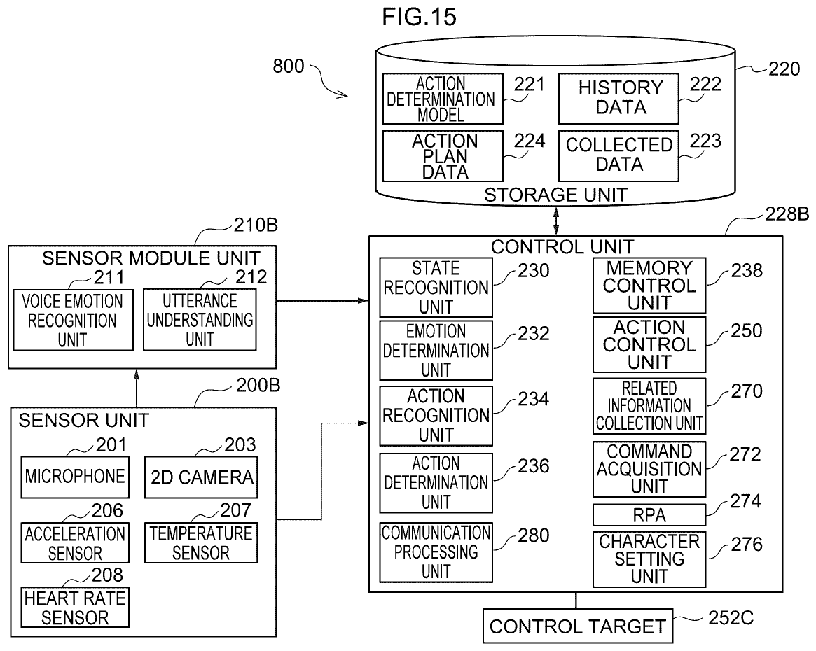

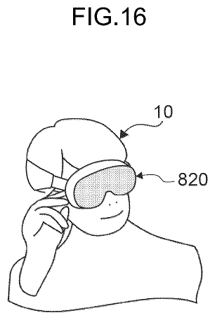

FIG. 15 is a functional block diagram of an agent system 800 configured using some or all of the functions of the action control system. The agent system 800 includes a sensor unit 200B, a sensor module unit 210B, a storage unit 220, a control unit 228B, and a control target 252C. The agent system 800 is implemented by, for example, a headset-type terminal 820 as illustrated in FIG. 16.

Further, the headset-type terminal 820 may function as each unit of the agent system 800 when a part of the headset-type terminal 820 (for example, the sensor module unit 210B, the storage unit 220, and the control unit 228B) is provided outside the headset-type terminal 820 (for example, a server) and the headset-type terminal communicates with the outside.

In the embodiment, the control unit 228B has the functions of determining an action of the avatar and generating display of the avatar to be presented to the user through the headset-type terminal 820.

As in the first embodiment, the emotion determination unit 232 of the control unit 228B determines an emotion value of the agent based on the state of the headset-type terminal 820, and substitutes the emotion value as an emotion value of the avatar. The emotion determination unit 232 may determine an emotion of the user or an emotion of the avatar representing an agent for interacting with the user.

As in the first embodiment, when an agent functioning as an avatar performs an autonomous process of autonomously acting, the action determination unit 236 of the control unit 228B determines, as an action of the avatar, any of multiple types of avatar actions including not acting, using at least one of the state of the user 10, the emotion of the user 10, the emotion of the avatar, or the state of electronic equipment (for example, the headset-type terminal 820) that controls the avatar, and the action determination model 221, at a predetermined timing. The action determination model 221 may be a data generation model capable of generating data according to input data.

Specifically, the action determination unit 236 inputs a text representing at least one of the state of the user 10, the state of the electronic equipment, the emotion of the user 10, or the emotion of the avatar, together with a text for inquiry about the action of the avatar to the sentence generation model, and determines the action of the avatar based on the output of the sentence generation model.

In addition, the action control unit 250 displays the avatar in the image display area of the headset-type terminal 820 as the control target 252C according to the determined action of the avatar. Furthermore, in a case in which the determined action of the avatar includes the utterance content of the avatar, the utterance content of the avatar is output from the speaker as the control target 252C by voice.

In particular, in a case in which the action determination unit 236 determines, as an action of the avatar, to dream, that is, to create an original event, the action control unit 250 controls the avatar to create an original event. That is, in a case in which the action determination unit 236 determines to dream, as an action of the avatar, the action determination unit 236 creates an original event obtained by combining multiple pieces of event data among pieces of data in the history data 222 by using the sentence generation model, as in the first embodiment. At this time, the action determination unit 236 creates the original event while randomly shuffling or exaggerating the past experience and conversation between the avatar and the user 10 or the family of the user 10 in the history data 222. Furthermore, based on the created original event, that is, the dream, the action determination unit 236 generates a dream image that is a collage of dreams by using the image generation model. In this case, a dream image may be generated based on one scene from the past memory stored in the history data 222, or a plurality of memories may be randomly shuffled and combined to generate a dream image. For example, in a case in which the action determination unit 236 ascertains that the user 10 camped in the forest from the history data 222, the action determination unit may generate a dream image indicating that the user camped on the riverside. Furthermore, for example, in a case in which the action determination unit 236 ascertains that the user 10 watched fireworks at a certain place from the history data 222, the action determination unit may generate a dream image indicating that the user watched fireworks at a completely different place. Furthermore, not only an image representing an event that has not actually occurred, such as a “dream”, but also an image representing what the avatar has seen and heard while the user 10 is not present may be generated as a dream image.

The action control unit 250 controls the avatar to generate a dream image. Specifically, an image of the avatar is generated such that the avatar draws the dream image generated by the action determination unit 236 on a canvas, a whiteboard, or the like in a virtual space. As a result, an appearance of the avatar drawing the dream image on a canvas, a whiteboard, or the like in the image display area is displayed in the headset-type terminal 820.

Note that the action control unit 250 may change the expression of the avatar or change the movement of the avatar according to the content of the dream. For example, in a case in which the content of the dream is a pleasant content, the expression of the avatar may be changed to an expression of pleasure, or the movement of the avatar may be changed as if the avatar is dancing with pleasure. Furthermore, the action control unit 250 may transform the avatar in accordance with the content of the dream. For example, the action control unit 250 may transform the avatar into an avatar imitating a character in the dream, or transform the avatar into an avatar imitating an animal, an object, or the like appearing in the dream.

Furthermore, the action control unit 250 may generate an image so as to cause the avatar to have a tablet terminal drawn in a virtual space and perform an operation of drawing the dream image on the tablet terminal. In this case, by transmitting the dream image displayed on the tablet terminal to the mobile terminal device of the user 10, it is possible to express an operation such as transmission of the dream image by e-mail from the tablet terminal to the mobile terminal device of the user 10 or transmission of the dream image to a messenger application as if the avatar is performing the operation. Furthermore, in this case, the user 10 can view the dream image displayed on his/her mobile terminal device.

Here, the avatar is, for example, a 3D avatar, and may be selected by the user from avatars prepared in advance, may be a virtual avatar of the user, or may be a favorite avatar generated by the user. To generate an avatar, image generative AI may be utilized to generate an avatar in multiple art styles such as photorealistic, cartoon, moe-style, and oil painting style.

Note that, although the case in which the headset-type terminal 820 is used has been described as an example in the above embodiment, the invention is not limited thereto, and an eyeglass-type terminal having an image display area for displaying an avatar may be used.

Furthermore, although the case in which the sentence generation model capable of generating a sentence according to input texts is used has been described as an example in the above embodiment, the invention is not limited thereto, and a data generation model other than the sentence generation model may be used. For example, a prompt including an instruction is input to the data generation model, and inference data such as voice data indicating a voice, text data indicating a text, and image data indicating an image is input thereto. The data generation model infers the input inference data according to the instruction indicated by the prompt, and outputs the inference result in a data format such as voice data and text data. Here, the inference refers to, for example, analysis, classification, prediction, and/or summary.

Furthermore, although the case in which the robot 100 recognizes the user 10 using a face image of the user 10 has been described in the above embodiment, the disclosed technology is not limited to this mode. For example, the robot 100 may recognize the user 10 using a voice uttered by the user 10, a mail address of the user 10, an ID of social media of the user 10, an ID card carried by the user 10 in which a wireless IC tag is built, or the like.

The robot 100 is an example of electronic equipment including an action control system. The application target of the action control system is not limited to the robot 100, and the action control system can be applied to various types of electronic equipment. Furthermore, the function of the server 300 may be implemented by one or more computers. At least some functions of the server 300 may be implemented by a virtual machine. Furthermore, at least some functions of the server 300 may be implemented in a cloud.

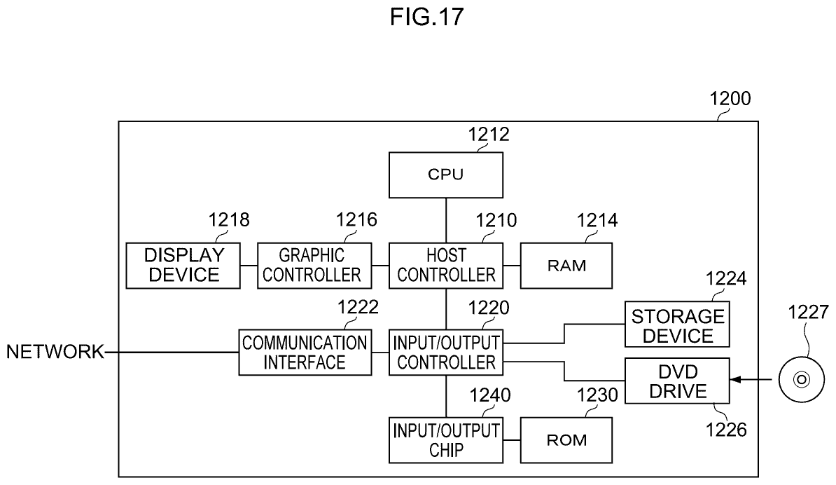

FIG. 17 schematically illustrates an example of a hardware configuration of a computer 1200 functioning as the smartphone 50, the robot 100, the server 300, and the agent systems 500, 700, and 800. A program installed in the computer 1200 can cause the computer 1200 to function as one or more “units” of a device according to the present embodiment, or cause the computer 1200 to execute an operation associated with the device according to the present embodiment or one or more “units” thereof, and/or cause the computer 1200 to execute a process according to the present embodiment or stages of the process. Such programs may be executed by a CPU 1212 to cause the computer 1200 to perform certain operations associated with some or all of the blocks in the flowcharts and block diagrams described in the present specification.

The computer 1200 according to the present embodiment includes the CPU 1212, a RAM 1214, and a graphic controller 1216, which are mutually connected by a host controller 1210. The computer 1200 also includes input/output units such as a communication interface 1222, a storage device 1224, a DVD drive 1226, and an IC card drive, which are connected to the host controller 1210 via an input/output controller 1220. The DVD drive 1226 may be a DVD-ROM drive, a DVD-RAM drive, or the like. The storage device 1224 may be a hard disk drive, a solid state drive, or the like. The computer 1200 also includes a ROM 1230 and legacy input/output units such as a keyboard, which are connected to the input/output controller 1220 via an input/output chip 1240.

The CPU 1212 operates according to programs stored in the ROM 1230 and the RAM 1214, thereby controlling each of the units. The graphics controller 1216 obtains image data generated by the CPU 1212 in a frame buffer or the like provided in the RAM 1214 or itself, and causes the image data to be displayed on a display device 1218.

The communication interface 1222 communicates with other electronic devices via a network. The storage device 1224 stores programs and data used by the CPU 1212 in the computer 1200. The DVD drive 1226 reads a program or data from the DVD-ROM 1227 or the like and provides the program or data to the storage device 1224. The IC card drive reads the program and data from the IC card and/or writes the program and data to the IC card.

The ROM 1230 stores therein a boot program executed by the computer 1200 at the time of activation and/or a program depending on hardware of the computer 1200. The input/output chip 1240 may also connect various input/output units to the input/output controller 1220 via a USB port, a parallel port, a serial port, a keyboard port, a mouse port, or the like.

Programs are provided by a computer-readable storage medium such as the DVD-ROM 1227 or an IC card. The programs are read from a computer-readable storage medium, installed in the storage device 1224, the RAM 1214, or the ROM 1230, which is also an example of a computer-readable storage medium, and executed by the CPU 1212. Information processing described in those programs is read by the computer 1200 and brings about cooperation between the programs and the various types of hardware resources. A device or a method may be configured by implementing an operation or processing of information according to use of the computer 1200.

For example, in a case in which communication is performed between the computer 1200 and an external device, the CPU 1212 may execute a communication program loaded in the RAM 1214 and instruct the communication interface 1222 to perform communication processing based on processing described in the communication program. Under control of the CPU 1212, the communication interface 1222 reads transmission data stored in a transmission buffer area provided in a recording medium such as the RAM 1214, the storage device 1224, the DVD-ROM 1227, or the IC card, transmits the read transmission data to the network, or writes reception data received from the network to a reception buffer area or the like provided on the recording medium.

In addition, the CPU 1212 may cause the RAM 1214 to read all or a necessary portion of a file or database stored in an external recording medium such as the storage device 1224, the DVD drive 1226 (DVD-ROM 1227), an IC card, or the like, and may execute various types of processing on data on the RAM 1214. Next, the CPU 1212 may write back the processed data to the external recording medium.

Various types of information such as various types of programs, data, tables, and databases may be stored in a recording medium and subjected to information processing. The CPU 1212 may execute various types of processing on the data read from the RAM 1214, including various types of operations, information processing, condition determination, conditional branching, unconditional branching, information search/replacement, and the like, which are described throughout the disclosure and specified in command sequences of a program, and writes back the results to the RAM 1214. In addition, the CPU 1212 may search for information in a file, a database, or the like in the recording medium. For example, in a case in which multiple entries each having an attribute value of a first attribute associated with an attribute value of a second attribute are stored in the recording medium, the CPU 1212 may search for an entry with the attribute value of the first attribute matching the specified condition from the multiple entries, read the attribute value of the second attribute stored in the entry, and thereby acquire the attribute value of the second attribute associated with the first attribute satisfying a predetermined condition.

The programs or software modules described above may be stored in a computer-readable storage medium on or near the computer 1200. Furthermore, a recording medium such as a hard disk or a RAM provided in a server system connected to a dedicated communication network or the Internet can be used as a computer-readable storage medium, thereby providing a program to the computer 1200 via the network.

The blocks in the flowcharts and block diagrams in the present embodiment may represent stages of a process in which an operation is performed or “units” of a device that are responsible for performing the operation. Certain stages and “units” may be implemented by a dedicated circuit, a programmable circuit provided with computer-readable instructions stored on a computer-readable storage medium, and/or a processor provided with computer-readable instructions stored on a computer-readable storage medium. The dedicated circuit may include a digital and/or analog hardware circuit, and may include an integrated circuit (IC) and/or a discrete circuit. The programmable circuit may include a reconfigurable hardware circuit including, for example, logical AND, logical OR, exclusive OR, NAND, NOR, and other logical operations, flip-flops, registers, and memory elements, such as a field programmable gate array (FPGA) and a programmable logic array (PLA).

A computer-readable storage medium may include any tangible device capable of storing instructions to be executed by a suitable device, such that a computer-readable storage medium having instructions stored thereon will comprise an article of manufacture including instructions that, when executed, create means for performing the operations specified in the flowcharts or block diagrams. Examples of the computer-readable storage medium may include an electronic storage medium, a magnetic storage medium, an optical storage medium, an electromagnetic storage medium, a semiconductor storage medium, and the like. More specific examples of the computer-readable storage medium may include a floppy (registered trademark) disk, a diskette, a hard disk, a random access memory (RAM), a read-only memory (ROM), an erasable programmable read-only memory (EPROM or flash memory), an electrically erasable programmable read-only memory (EEPROM), a static random access memory (SRAM), a compact disc read-only memory (CD-ROM), a digital versatile disk (DVD), a Blu-Ray (registered trademark) disk, a memory stick, an integrated circuit card, and the like.

The computer-readable instructions may include any of source codes or object codes written in any combination of one or more programming languages, including assembler instructions, instruction-set-architecture (ISA) instructions, machine instructions, machine-dependent instructions, microcode, firmware instructions, state-setting data, or an object-oriented programming language such as Smalltalk, JAVA (registered trademark), C++, or the like, and conventional procedural programming languages, such as the ‘C’ programming language or similar programming languages.

The computer readable instructions may be provided to processors of general purpose computers, special purpose computers, or other programmable data processing devices, or programmable circuits, either locally or over a wide area network (WAN), such as a local area network (LAN), the Internet, or the like, to cause the processors or programmable circuits of the general purpose computers, special purpose computers, or other programmable data processing devices to execute the computer readable instructions to generate means for the processors or programmable circuits to perform the operations specified in the flowcharts or block diagrams. Examples of the processor include a computer processor, a processing unit, a microprocessor, a digital signal processor, a controller, a microcontroller, and the like.

### Sixth Embodiment

In the autonomous processing in the present embodiment, an equipment operation (a robot action in a case in which the electronic equipment is the robot 100) determined by the action determination unit 236 includes proposing an activity. Then, in a case in which the action determination unit 236 determines to propose an activity as an action of the electronic equipment (action of the robot), the action determination unit 236 determines an action of the user 10 to propose based on the event data.

In a case in which it is determined that “(3) The robot speaks to the user”, that is, the robot 100 utters, as a robot action, the action determination unit 236 determines the utterance content of the robot corresponding to the user state and the user's emotion or the robot's emotion using the sentence generation model. At this time, the action control unit 250 causes a speaker included in the control target 252 to output a voice representing the determined utterance content of the robot. Note that, in a case in which the user 10 is absent around the robot 100, the action control unit 250 stores the determined utterance content of the robot in the action plan data 224 without outputting a voice representing the determined utterance content of the robot.

As described above, in a case in which the action determination unit 236 determines that “(5) The robot proposes an activity”, that is, an action of the user 10 is proposed, as a robot action, the action determination unit can determine the proposed action of the user using the sentence generation model based on the event data stored in the history data 222. At this time, the action determination unit 236 may propose “play”, “learning”, “cooking”, “traveling”, or “shopping”, as an action of the user 10. In this manner, the action determination unit 236 can determine the type of activity to be proposed. Furthermore, in a case in which the action determination unit 236 proposes “play”, the action determination unit can propose “Let's go on a picnic on the weekend.”. Furthermore, in a case in which the action determination unit 236 proposes “cooking”, the action determination unit can also propose “Let's have curry rice for the dinner menu for this evening.”. Furthermore, in a case in which the action determination unit 236 proposes “shopping”, the action determination unit can also propose “Let's go to [Name] shopping mall.”. In this manner, the action determination unit 236 can determine the details of the activity to propose, such as “when”, “where”, and “what”. Note that, in determining the type and details of such an activity, the action determination unit 236 can learn the past experience of the user 10 by using the event data stored in the history data 222. Then, the action determination unit 236 may propose an action that the user 10 enjoyed in the past, an action that the user 10 seems to like from the preferences and tastes of the user 10, a new action that the user 10 has not experienced before.

Particularly, in a case in which the action determination unit 236 determines to propose an activity as an action of the avatar, the action control unit 250 is preferably caused to control the avatar such that the action of the user to propose is determined based on the event data.

Specifically, in a case in which the action determination unit 236 determines to propose an activity, that is, an action of the user 10 is proposed, as an avatar action, the action determination unit can determine the proposed action of the user using the sentence generation model based on the event data stored in the history data 222. At this time, the action determination unit 236 may propose “play”, “learning”, “cooking”, “traveling”, “dinner menu of tonight”, “picnic”, or “shopping”, as an action of the user 10. In this manner, the action determination unit 236 can determine the type of activity to be proposed. Furthermore, in a case in which the action determination unit 236 proposes “play”, the action determination unit can propose “Let's go on a picnic on the weekend.”. Furthermore, in a case in which the action determination unit 236 proposes “cooking”, the action determination unit can also propose “Let's have curry rice for the dinner menu for this evening.”. Furthermore, in a case in which the action determination unit 236 proposes “shopping”, the action determination unit can also propose “Let's go to [Name] shopping mall.”. In this manner, the action determination unit 236 can determine the details of the activity to propose, such as “when”, “where”, and “what”. Note that, in determining the type and details of such an activity, the action determination unit 236 can learn the past experience of the user 10 by using the event data stored in the history data 222. Then, the action determination unit 236 may propose at least one of the action that the user 10 enjoyed in the past, the action that the user 10 seems to like from the preferences and tastes of the user 10, or a new action that the user 10 has not experienced before.

Furthermore, in a case in which an activity is proposed as an avatar action, the action control unit 250 may operate the avatar so as to perform the proposed activity, and display the avatar in the image display area of the headset-type terminal 820 as the control target 252C.

### Seventh Embodiment

In the autonomous processing in the present embodiment, an equipment operation (a robot action in a case in which the electronic equipment is the robot 100) determined by the action determination unit 236 includes comforting the user 10. Then, in a case in which the action determination unit 236 determines to comfort the user 10 as an action of the electronic equipment (action of the robot), the action determination unit determines an utterance content corresponding to the user state and the emotion of the user 10.

For example, multiple types of the robot actions include the following (1) to (11).
(1) The robot does nothing.
(2) The robot dreams.
(3) The robot speaks to the user.
(4) The robot creates a picture diary.
(5) The robot proposes an activity.
(6) The robot proposes a person whom the user should meet.
(7) The robot introduces news that the user is interested in.
(8) The robot edits pictures and videos.
(9) The robot studies with the user.
(10) The robot evokes a memory.
(11) The robot comforts the user.

In a case in which the action determination unit 236 determines that “(11) The robot comforts the user.”, that is, the robot 100 makes an utterance to comfort the user 10, as a robot action, the action determination unit determines the utterance content corresponding to the state of the user 10 and the emotion of the user 10. For example, in a case in which the state of the user 10 satisfies the condition “being depressed”, the action determination unit 236 determines “(11) The robot comforts the user” as the robot action. Note that the state of the user 10 being depressed may be recognized, for example, by performing processing related to perception using the analysis results of the sensor module unit 210. In such a case, the action determination unit 236 determines the utterance content corresponding to the state of the user 10 and the emotion of the user 10. As an example, the action determination unit 236 may determine an utterance content such as “What's wrong? What happened at school?”, “What are you worried about?”, or “You can talk to me anytime.” in a case in which the user 10 is depressed. Then, the action control unit 250 may cause a voice expressing the determined utterance content of the robot 100 to be output from a speaker included in the control target 252. In this manner, the robot 100 can provide the user 10 (child, family member, etc.) with an opportunity to verbalize and release his/her emotion to the outside by listening to the speech of the user 10. Thus, the robot 100 can ease the feeling of the user 10 by helping the user calm the feeling, organizing the problem points, finding a clue to the solution, or the like.

In particular, in a case in which the action determination unit 236 determines to comfort the user as an action of the avatar, for example, it is preferable that the action control unit 250 is caused to control the avatar so as to listen to the story of the depressed child, a family member, or the like and comfort the depressed child or the family member.

### Eighth Embodiment

In the autonomous processing in the present embodiment, an equipment operation (a robot action in a case in which the electronic equipment is the robot 100) determined by the action determination unit 236 includes presenting a question to the user 10. Then, in a case in which the action determination unit 236 determines to present a question to the user 10 as an action of the electronic equipment (action of the robot), a question to be presented to the user 10 is created.

For example, multiple types of the robot actions include the following (1) to (11).
(1) The robot does nothing.
(2) The robot dreams.
(3) The robot speaks to the user.
(4) The robot creates a picture diary.
(5) The robot proposes an activity.
(6) The robot proposes a person whom the user should meet.
(7) The robot introduces news that the user is interested in.
(8) The robot edits pictures and videos.
(9) The robot studies with the user.
(10) The robot evokes a memory.
(11) The robot presents a question to the user.

In a case in which the action determination unit 236 determines that “(11) The robot presents a question to the user.”, that is, the robot 100 makes an utterance to present a question to the user 10, as a robot action, a question to be presented to the user 10 is created. For example, the action determination unit 236 may create a question to be presented to the user 10 based on at least one of the interaction history of the user 10 or the personal information of the user 10. As an example, in a case in which it is estimated that the user 10's weak subject is math from the interaction history of the user 10, the action determination unit 236 may create the question “What is the answer to 7×7?”. Then, the action control unit 250 may cause a voice expressing the created question to be output from the speaker included in the control target 252. Next, in a case in which the user 10 answers “49”, the action determination unit 236 may determine an utterance content “Correct. Good work, great!”. Then, in a case in which it is estimated from the emotion of the user 10 that the user was interested in the presented question, the action determination unit 236 may create a new question having the same question trend. As another example, in a case in which the age of the user is found to be 10 from the personal information of the user 10, the action determination unit 236 may create a question “What is the capital city of the United States of America?” as a question corresponding to that age. Then, the action control unit 250 may cause a voice expressing the created question to be output from the speaker included in the control target 252. Next, in a case in which the user 10 answers “New York”, the action determination unit 236 may determine an utterance content “Too bad. The correct answer is Washington D.C.”. Then, in a case in which it is estimated from the emotion of the user 10 that the user is not interested in the presented question, the action determination unit 236 may change the question trend and create a new question. In this manner, the robot 100 can boost the learning motivation of the user 10 by spontaneously presenting a question that feels like a game to help the user 10, who is a child, for example, enjoy studying, and providing positive feedback or expressing satisfaction according to the answer of the user 10.

In particular, in a case in which the action determination unit 236 determines to present a question to the user as an action of the avatar, it is preferable to cause the action control unit 250 to control the avatar to create a question to be presented to the user.

Specifically, in a case in which the action determination unit 236 determines that “The avatar presents a question to the user.”, that is, the avatar makes an utterance to present a question to the user 10, as an avatar action, a question to be presented to the user 10 is created. For example, the action determination unit 236 may create a question to be presented to the user 10 based on at least one of the interaction history of the user 10 or the personal information of the user 10. As an example, in a case in which it is estimated that the user 10's weak subject is math from the interaction history of the user 10, the action determination unit 236 may create the question “What is the answer to 7×7?”. In response to this, the action control unit 250 may cause a voice expressing the created question to be output from the speaker as the control target 252C. Next, in a case in which the user 10 answers “49”, the action determination unit 236 may determine an utterance content “Correct. Good work, great!”. Then, in a case in which it is estimated from the emotion of the user 10 that the user was interested in the presented question, the action determination unit 236 may create a new question having the same question trend. As another example, in a case in which the age of the user is found to be 10 from the personal information of the user 10, the action determination unit 236 may create a question “What is the capital city of the United States of America?” as a question corresponding to that age. In response to this, the action control unit 250 may cause a voice expressing the created question to be output from the speaker as the control target 252C. Next, in a case in which the user 10 answers “New York”, the action determination unit 236 may determine an utterance content “Too bad. The correct answer is Washington D.C.”. Then, in a case in which it is estimated from the emotion of the user 10 that the user is not interested in the presented question, the action determination unit 236 may change the question trend and create a new question. In this manner, the avatar in augmented reality (AR) or virtual reality (VR) can boost the learning motivation of the user 10 by spontaneously presenting a question that feels like a game to help the user 10, who is a child, for example, come to like studying, and providing positive feedback or expressing satisfaction according to the answer of the user 10.

Furthermore, in a case in which a question is presented to the user as an avatar action, the action control unit 250 may operate the avatar so as to present a created question to the user, and display the avatar in the image display area of the headset-type terminal 820 as the control target 252C.

### Ninth Embodiment

In the autonomous processing in the present embodiment, an equipment operation (a robot action in a case in which the electronic equipment is the robot 100) determined by the action determination unit 236 includes teaching music. Then, in a case in which the action determination unit 236 determines to teach music as an action of the electronic equipment (action of the robot), a sound generated by the user 10 is evaluated.

For example, multiple types of the robot actions include the following (1) to (11).
(1) The robot does nothing.
(2) The robot dreams.
(3) The robot speaks to the user.
(4) The robot creates a picture diary.
(5) The robot proposes an activity.
(6) The robot proposes a person whom the user should meet.
(7) The robot introduces news that the user is interested in.
(8) The robot edits pictures and videos.
(9) The robot studies with the user.
(10) The robot evokes a memory.
(11) The robot teaches music.

In a case in which the action determination unit 236 determines that “(11) The robot teaches music”, that is, the robot 100 makes an utterance to teach music to the user 10, as a robot action, a sound generated by the user 10 is evaluated. Note that the “sound generated by the user 10” mentioned herein may be interpreted to include various sounds generated in association with an action of the user 10, such as the singing voice of the user 10, sound of a musical instrument played by the user 10, or a tapping sound of the user 10. For example, in a case in which it is recognized that the user 10 is singing, playing a musical instrument, or dancing from an action of the user 10, the action determination unit 236 determines that “(11) The robot teaches music.” as a robot action. In such a case, the action determination unit 236 may evaluate at least one of a singing voice, a sound of a musical instrument, or a sense of rhythm of a tapping sound, a pitch, or an intonation of the user 10. Then, the action determination unit 236 may determine an utterance content such as “The rhythm is inconsistent.”, “Your pitch is off”, or “Put more feeling into it.” according to the evaluation result. Then, the action control unit 250 may cause a voice expressing the determined utterance content of the robot 100 to be output from a speaker included in the control target 252. As described above, even if there is no inquiry from the user 10, the robot 100 can spontaneously evaluate the sound generated by the user 10 and point out the sense of rhythm, a difference in pitch, and the like, and thus can play the role of a music teacher for the user 10.

In particular, in a case in which the action determination unit 236 determines to teach music as an action of the avatar, it is preferable to cause the action control unit 250 to control the avatar to evaluate the sound generated by the user.

Specifically, in a case in which the action determination unit 236 determines that “The avatar teaches music.”, that is, the avatar makes an utterance to teach music to the user 10, as an avatar action, a sound generated by the user 10 is evaluated. Note that the “sound generated by the user 10” mentioned herein may be interpreted to include various sounds generated in association with an action of the user 10, such as the singing voice of the user 10, sound of a musical instrument played by the user 10, or a tapping sound of the user 10. For example, in a case in which it is recognized that the user 10 is singing, playing a musical instrument, or dancing from the action of the user 10, the action determination unit 236 determines “The avatar teaches music.” as an action of the avatar. In such a case, the action determination unit 236 may evaluate at least one of a singing voice, a sound of a musical instrument, or a sense of rhythm of a tapping sound, a pitch, or an intonation of the user 10. Then, the action determination unit 236 may determine an utterance content such as “The rhythm is inconsistent.”, “Your pitch is off”, or “Put more feeling into it.” according to the evaluation result. Then, the action control unit 250 may cause a voice expressing the determined utterance content of the avatar to be output from a speaker as the control target 252C. In this manner, the avatar in augmented reality (AR) or virtual reality (VR) can spontaneously evaluate the sound generated by the user 10 and utter the evaluation result to point out the sense of rhythm, a difference in pitch, or the like, even without an inquiry from the user 10, and thus can play the role of a music teacher for the user 10.

Furthermore, in a case in which the avatar teaches music as an avatar action, the action control unit 250 may operate the avatar so as to utter the evaluation results about the sound generated by the user, and display the avatar in the image display area of the headset-type terminal 820 as the control target 252C.

### Tenth Embodiment

In the autonomous processing in the present embodiment, the robot 100 as an agent performs autonomous processing. More specifically, the autonomous processing in which the robot 100 performs an action is performed based on the past history (there may be no history) of the robot 100 and action monitoring of the user 10 regardless of whether the user 10 is present.

The robot 100 as an agent spontaneously and periodically detects states of the user 10. For example, the robot 100 reads a text of a textbook of a school or a cram school that the user 10 attends, is made to think about new questions by using a sentence generation model using AI, and generates a question that matches a preset target deviation value (for example, 50, 60, 70, and the like) of the user 10.

The robot 100 may determine the subject of the question to be presented based on the behavior history of the user 10. That is, if it is found from the action history that the user 10 is studying math, the robot 100 generates math questions and presents the generated questions to the user 10.

In particular, in a case in which the action determination unit 236 determines to present a question to the user 10 as an action of the avatar as described in the first embodiment, it is preferable to generate a question that matches a preset target deviation value (for example, 50, 60, 70, and the like) of the user 10 and cause the action control unit 250 to control the avatar to present the generated question.

When presenting the question to the user 10, the action control unit 250 may control the avatar such that the avatar transforms its appearance to a specific person, for example, a parent, a friend, a school teacher, a cram school lecturer, or the like. In particular, the avatar appearance for a school teacher and a cram school lecturer may be transformed for each subject. For example, the action control unit 250 controls the avatar such that the avatar transforms into a foreigner for the English subject and into a person wearing a white gown for the science subject. In this case, the action control unit 250 may cause the avatar to read the question aloud, or may cause the avatar to hold the paper on which the question sentence is written. Furthermore, in this case, the action control unit 250 may control the avatar so as to change the expression based on the emotion value of the user 10 determined by the emotion determination unit 232. For example, if the emotion value of the user 10 is positive such as “joy” or “pleasure”, the action control unit 250 may change the expression of the avatar to be bright, and if the emotion value of the user 10 is negative such as “anxiety” or “sadness”, the action control unit 250 may change the expression of the avatar to be encouraging the user 10.

Furthermore, when a question is presented to the user 10, the action control unit 250 may control the avatar so as to transform the avatar into the form of a blackboard or a whiteboard on which the question is written. Furthermore, in a case in which a time limit is set for the answer to the question, the action control unit 250 may cause the avatar to transform the appearance into a clock indicating the remaining time until the time limit when the question is presented to the user 10. Furthermore, when the question is presented to the user 10, the action control unit 250 may perform control such that a virtual blackboard or whiteboard and a virtual clock indicating the remaining time until the time limit are displayed in addition to the human-looking avatar. In this case, after the avatar having the whiteboard presents the question to the user 10, the avatar can switch the whiteboard to a clock and notify the user 10 of the remaining time.

The action control unit 250 may control the action of the avatar such that the avatar takes an action of praising the user 10 in a case in which the user 10 gives the correct answer to the question presented by the avatar. In addition, the action control unit 250 may control the action of the avatar such that the avatar takes an action of encouraging the user 10 in a case in which the user 10 fails to give the correct answer to the question presented by the avatar.

Furthermore, the action control unit 250 may control the action of the avatar so as to provide a hint for the answer in a case in which the user 10 is pondering, struggling to find an answer to the question presented by the avatar.

Note that, in a case in which the action control unit 250 changes the action of the avatar, the expression of the avatar can be changed according to not only the emotion value of the user 10 but also the emotion value of the agent who is the avatar, the target deviation value of the user 10, and the like. Furthermore, the avatar currently displayed in response to a predetermined action of the user 10 to the question presentation may be replaced with another avatar. For example, the appearance of the lecturer avatar may be transformed into an angel avatar, triggered by all the correct answers to the questions presented by the avatar, or the avatar having a gentle appearance may be transformed into a tough-looking avatar, triggered by the target deviation value getting lowered due to continuous wrong answers to the questions by the avatar.

### Eleventh Embodiment

In the autonomous processing in the present embodiment, the robot 100 includes processing of spontaneously or periodically identifying, at an arbitrary timing, a state of the user participating in a specific competition or a state of the player of the opposing team, particularly identifying the features of the player, and giving advice on the specific competition to the user based on the identified result. Here, the specific competition may be a sport performed by a team including a plurality of people, such as volleyball, soccer, or rugby. Furthermore, the user participating in the specific competition may be a player performing the specific competition or support staff such as a manager or a coach of a specific team performing the specific competition. Furthermore, the features of the player refer to information related to the abilities related to the competition and the current or recent condition of the player, such as the habit, movement, the number of mistakes, unskillful movement, and reaction speed of the player.

For example, multiple types of the robot actions include the following (1) to (11).
(1) The robot does nothing.
(2) The robot dreams.
(3) The robot speaks to the user.
(4) The robot creates a picture diary.
(5) The robot proposes an activity.
(6) The robot proposes a person whom the user should meet.
(7) The robot introduces news that the user is interested in.
(8) The robot edits pictures and videos.
(9) The robot studies with the user.
(10) The robot evokes a memory.
(11) The robot gives advice to the user participating in a specific competition.

In a case in which the action determination unit 236 determines that, as a robot action, “(11) The robot gives advice to the user participating in a specific competition.”, that is, the robot gives advice on a specific competition that the user is participating in to the user such as a player or a coach participating in the specific competition, the action determination unit 236 first specifies features of a plurality of players participating in the competition that the user is participating in.

In order to specify the features of the above-described player, the action determination unit 236 includes an image acquisition unit that captures an image of the competition space in which the specific competition in which the user is participating is being performed. The image acquisition unit can realize the competition space, for example, by using a part of the sensor unit 200 described above. Here, the competition space may include a space corresponding to each competition, for example, a volleyball court, a soccer ground, or the like. Furthermore, the competition space may include a region around the above-described court or the like. It is preferable that the installation position of the robot 100 may be considered such that the competition space can be overlooked by the image acquisition unit.

Furthermore, the action determination unit 236 further includes a feature identifying unit capable of identifying features of a plurality of players in an image acquired by the image acquisition unit described above. The feature identifying unit can identify features of a plurality of players by analyzing past competition data, collecting and analyzing information regarding each player from social media or the like, or combining one or more of these methods, by using a method similar to the method of determining an emotion value in the emotion determination unit 232. Note that the above-described image acquisition unit and feature identifying unit may be collected and stored as a part of the collected data 223 by the related information collection unit 270. In particular, the information such as the past competition data of the players described above may be collected by the related information collection unit 270.

Once the features of a player in a particular competition, e.g. volleyball, can be identified, the match can be advantageously led by reflecting that identification result in the team's strategy. Specifically, a player with a large number of mistakes or a player with a specific habit can be a weak point of the team. Therefore, in the present embodiment, advice for advantageously leading the competition, specifically, the feature of each player identified by the action determination unit 236 is given to the user, for example, the coach of one team during the competition, and thus, advice is given to the user.

In consideration of the above points, the player whose features are identified by the feature identifying unit may be a player belonging to a specific team among a plurality of players in the competition space. More specifically, the specific team may be a team different from the team to which the user belongs, in other words, the opponent team. The robot 100 scans the features of each player of the opponent team, identifies a player with a specific habit or a player who frequently makes mistakes, and provides the user with information regarding the features of the player as advice, which can help the user create effective strategies.

If the user uses advice provided by the robot 100 during a match in a team-versus-team competition, it can be expected that the user predominates the match. Specifically, for example, if it is possible to identify the player or the like with many mistakes during the competition based on the advice from the robot 100 and adopt the strategy of concentrating on the position of the player to tackle the player, the team can get closer to a win.

The advice by the action determination unit 236 described above is not initiated by a request from the user, and is preferably performed autonomously by the robot 100. Specifically, for example, it is preferable to detect when the coach who is the user is in trouble, when the team to which the user belongs is about to lose, when a member of the team to which the user belongs is having a conversation to indicate that he/she wants advice, and the like, and it is preferable that the robot 100 itself should make an utterance.

A specific method for the action control unit 250 to cause the avatar to perform a desired operation will be described below. First, a state including features of a plurality of players participating in a competition in which the user is participating is detected. The detection of the features of the plurality of players can be realized by the image acquisition unit of the action determination unit 236 described above. The emotion of the player or the like can be detected spontaneously or periodically by the action control unit 250, for example. At this time, the image acquisition unit is preferably arranged at a position where the user or the like is playing a competition, that is, at a position where the entire competition space can be overlooked. In consideration of this point, the image acquisition unit can be constituted by, for example, a camera with a communication function that can be installed at an arbitrary position independently of the headset-type terminal 820.

To analyze the features of the plurality of players in the image acquired by the image acquisition unit, the feature identifying unit of the action determination unit 236 described above is used. The features of each player analyzed by the feature identifying unit can be reflected in the control of the avatar by the action control unit 250.

In the agent system 800 according to the present embodiment, the action control unit 250 controls the avatar based on at least the features identified by the feature identifying unit. How the action control unit 250 specifically controls the avatar is not particularly limited as long as predetermined advice can be provided to the user by the control. Although the control may mainly include causing the avatar to utter, it is also possible to make it easier for the user to understand the meaning by adopting other operations alone or in combination with an utterance or the like. Therefore, some examples of control contents of the avatar by the action control unit 250 will be described below. Note that, in the following description, it is assumed that the agent system 800 is used to give advice to the coach of one team participating in a volleyball match on the match that the team is participating in via the headset-type terminal 820 worn by the coach.

When the action determination unit 236 determines to give advice on the volleyball match that the user (coach) is participating in, as an action of the avatar, the action control unit 250 starts to provide the advice through the avatar. As a method of providing advice, for example, by reflecting, in the avatar, the feature of a specific player among a plurality of players, information regarding the state of the specific player can be provided to the user. Describing a more specific example, when a player with many mistakes or a player with a particular habit among the players of the opponent team is identified by the feature identifying unit, the action control unit 250 transforms the appearance of the avatar to an appearance resembling the specified player and reflects the features identified by the feature identifying unit on the expression, movements, and the like. As a result, the state of the specific player can be visually conveyed to the user. In addition, if the state of the specific player is conveyed to the user by causing the avatar to make an utterance using the output of the action determination model 221, the user can more accurately ascertain the state of the specific competitor.

For example, when it is specified that the specific player of the opponent team makes more mistakes than the other player, it is possible to immediately notify the user that the specific player is likely to make a mistake by making the avatar's complexion displayed to resemble the specific player bluish or making the avatar perform an action when making a mistake. In addition, when the avatar uses the output of the action determination model 221 together with such avatar display to make an utterance such as “The player with the back number 7 of the opponent team makes many mistakes”, the coach as the user can plan a strategy in consideration of the situation of the player.

Furthermore, for example, in a case in which it has been ascertained that the opponent team has a player with a specific habit, it is possible to immediately notify the user of the habit of the specific player by making the avatar resemble the specific player and causing the avatar to perform a movement that the player is not good at. In addition, when the avatar uses the output of the action determination model 221 together with such avatar display to make an utterance such as “The player with the back number 5 of the opponent team is not good at receiving”, the coach as the user can plan a strategy in consideration of the situation of the player.

Further, when the action determination unit 236 determines to give advice on the volleyball match that the user (coach) is participating in, as an action of the avatar, the action control unit 250 can reflect, in the avatar, information of the uniform to be worn during the specific competition. Specifically, the action control unit 250 can reflect, in the avatar, information of a volleyball uniform on which advice is given via the avatar, that is, to cause the avatar to wear the uniform. The uniform worn by the avatar may be a general uniform used for volleyball prepared in advance, or may be a uniform of a team to which the user belongs or a uniform of the opponent team. The information on the uniform of the team to which the user belongs and the uniform of the opponent team may be generated by, for example, analyzing the image acquired by the image acquisition unit, or may be registered in advance by the user.

As described above, reflecting the uniform information in the avatar makes it easier for the user to understand the information provided by the avatar. In the above example, it can be easily understood that the information provided from the avatar relates to a volleyball game that the user is participating in. In addition, as in the example described above, when the avatar is displayed to look similar to a specific player, the uniform is set to be similar to that worn by the specific player, so it becomes easier for the user to recognize which player the avatar is displayed to be similar to.

In the above-described example, the case in which the avatar is displayed to look similar to a specific player has been exemplified, but the specific player is not limited to one player. Similarly, the number of avatars displayed in the image display area of the electronic equipment is not particularly limited. Therefore, the action determination unit 236 can also reflect the features, uniforms, and the like of all the players of the opponent team of the user as a specific competitor in a plurality of avatars and display the avatars.

Note that, although the case in which the headset-type terminal 820 is used has been described as the electronic equipment in the above embodiment, the invention is not limited thereto, and, for example, an eyeglass-type terminal having an image display area for displaying an avatar may be used.

### Twelfth Embodiment

The state of the user may include an action tendency of the user. The action tendency may be interpreted as an action tendency of a hyperactive or impulsive user, such as a user frequently running up stairs, a user frequently climbing or trying to climb a dresser, or a user frequently climbing on a window edge to open the window. In addition, the action tendency may be interpreted as a tendency of an action with hyperactivity or impulsiveness, such as a user frequently walking on or trying to walk on a fence, or a user frequently walking on a roadway or entering a roadway from a sidewalk.

Furthermore, in the autonomous processing, the agent may ask a generative AI about the detected state or action of the user, and store the answer of the generative AI to the question and the detected action of the user in association with each other. At this time, the agent may store action contents for correcting the action in association with the answer.

Information in which the answer of the generative AI to the question, the detected action of the user, and the action content for correcting the action are associated with each other may be recorded as table information in a storage medium such as a memory. The table information may be interpreted as specific information recorded in the storage unit.

Furthermore, in the autonomous processing, an action plan of the robot 100 for calling attention to the state or action of the user may be set based on the detected action of the user and the stored specific information.

As described above, the agent can record table information in which the answer of the generative AI corresponding to the state or action of the user is associated with the detected state or action of the user in the storage medium. Hereinafter, an example of contents stored in the table will be described.

(1. A Case in which the User Tends to Frequently Run on Stairs)

In the case of this tendency, the agent itself asks the generative AI a question “What other things is the child who performs such an action likely to do?”. In a case in which the answer of the generative AI to this question is, for example, “The user is likely to stumble on the stairs”, the agent may store the action of the user running on the stairs and the answer of the generative AI in association with each other. In addition, the agent may store an action content for correcting the action in association with the answer.

The action content for correcting the action may include at least one of execution of a gesture for correcting the dangerous action of the user and reproduction of a voice for correcting the action.

The gesture for correcting the dangerous action may include a body gesture and a hand gesture to guide the user to a specific place, a body gesture and a hand gesture to make the user remain in that place, and the like. The specific place may include a place other than the place where the user is currently located, for example, the vicinity of the robot 100, a space of a window at the indoor side, or the like.

The voice for correcting the dangerous action may include a voice saying “Stop” or “[Name], it's dangerous, so don't move”, or the like. The voice for correcting the dangerous action may include a voice saying “Do not run” or “stay still”, or the like.

(2. A Case in which the User Tends to Frequently Stay on or Try to Climb a Chest of Drawers)

In the case of this tendency, the agent asks a question to the generative AI in the same manner as described above. In a case in which an answer of the generative AI to the question is, for example, “The user may fall from the chest of drawers” or “The user may be caught in the door of the chest of drawers”, the agent may store the action of the user who is on the chest of drawers or trying to climb on the chest of drawers in association with the answer of the generative AI. In addition, the agent may store an action content for correcting the action in association with the answer.

(3. A Case in which the User Frequently Tends to Climb on a Window Edge to Open the Window)

In the case of this tendency, the agent asks a question to the generative AI in the same manner as described above. In a case in which an answer of the generative AI to the question is, for example, “The user may put his/her face out of the window” or “The user may be caught in the window”, the agent may store the action of the user climbing on the window edge to open the window in association with the answer of the generative AI. In addition, the agent may store an action content for correcting the action in association with the answer.

(4. A Case in which the User Frequently Walks on or Tries to Climb on the Fence)

In the case of this tendency, the agent asks a question to the generative AI in the same manner as described above. In a case in which an answer of the generative AI to the question is, for example, “The user may fall from the fence” or “The user may be hurt by the unevenness of the wall”, the agent may store the action of the user who is walking on or trying to climb on the fence in association with the answer of the generative AI. In addition, the agent may store an action content for correcting the action in association with the answer.

(5. A Case in which the User Frequently Walks on a Roadway or Enters a Roadway from a Sidewalk)

In the case of this tendency, the agent asks a question to the generative AI in the same manner as described above. In a case in which an answer of the generative AI to the question is, for example, “You may cause a traffic accident” or “You may cause a traffic jam”, the agent may store the action of the user who is walking on a roadway or has entered a roadway from a sidewalk in association with the answer of the generative AI. In addition, the agent may store an action content for correcting the action in association with the answer.

As described above, in the autonomous processing, a table in which the answer of the generative AI corresponding to the state or action of the user, the content of the state or action, and the action content for correcting the state or action are associated with each other may be recorded in a storage medium such as a memory.

Furthermore, in the autonomous processing, after the table is recorded, the action of the user is autonomously or periodically detected, and an action plan of the robot 100 that urges the user to pay attention may be set based on the detected action of the user and the content of the stored table. Specifically, the action determination unit 236 of the robot 100 may cause the action control unit 250 to operate the robot 100 to implement a first action content for correcting the action of the user based on the detected action of the user and the content of the stored table. Hereinafter, an example of the first action content will be described.

(1. A Case in which the User Tends to Frequently Run on Stairs)

In a case in which the action determination unit 236 detects the user running up the stairs, the action determination unit 236 may cause the action control unit 250 to operate the robot 100 such that a body gesture and a hand gesture to guide the user to a place other than the stairs, a body gesture and a hand gesture to make the user remain in that place, and the like are executed as the first action content for correcting the action.

Furthermore, the action determination unit 236 can reproduce, as the first action content for correcting the action, a voice for guiding the user to a place other than the stairs, a voice for making the user remain in that place, or the like. The voice may include “[Name], it's dangerous, so don't run”, “Don't move”, “Don't run”, “Stay still”, or the like.

(2. A Case in which the User Tends to Frequently Stay on or Try to Climb a Chest of Drawers)

The action determination unit 236 may cause the action control unit 250 to operate the robot 100 so as to perform a body gesture and a hand gesture to make the user who is on the chest of the drawers or about to climb on the chest of the drawers remain still in that place, or a body gesture and a hand gesture to move the user to a place other than the current place.

(3. A Case in which the User Frequently Tends to Climb on a Window Edge to Open the Window)

The action determination unit 236 may cause the action control unit 250 to operate the robot 100 so as to perform a body gesture and a hand gesture to make the user who is at the window edge or placing his/her hand on the window at the window edge remain still in that place, or a body gesture and a hand gesture to move the user to a place other than the current place.

(4. A Case in which the User Frequently Walks on or Tries to Climb on the Fence)

The action determination unit 236 may cause the action control unit 250 to operate the robot 100 so as to perform a body gesture and a hand gesture to make the user walking on a fence or about to climb on the fence remain still in that place, or a body gesture and a hand gesture to move the user to a place other than the current place.

(5. A Case in which the User Frequently Walks on a Roadway or Enters a Roadway from a Sidewalk)

The action determination unit 236 may cause the action control unit 250 to operate the robot 100 so as to perform a body gesture and a hand gesture to make the user walking on a roadway or having entered the roadway from the sidewalk remain still in that place, or a body gesture and a hand gesture to move the user to a place other than the current place.

In a case in which, after the robot 100 performs a gesture that is the first action content or after the robot reproduces a voice that is the first action content, the action determination unit 236 determines whether or not the action of the user has been corrected by detecting the action of the user, and in a case in which the action of the user has been corrected, the action determination unit 236 may cause the action control unit 250 to operate the robot 100 so as to implement a second action content that is different from the first action content.

The case in which the action of the user has been corrected may be interpreted as a case in which, as a result of execution of the operation of the robot 100 according to the first action content, the user has stopped dangerous actions and behaviors or the dangerous situation has been resolved.

The second action content may include reproduction of at least one of a voice praising the action of the user and a voice expressing gratitude for the action of the user.

The voice praising the action of the user may include a voice indicating “Are you OK? Well listened”, “Good work, great”, or the like. The voice expressing gratitude for the action of the user may include a voice saying “Thank you for coming”.

In a case in which, after the robot 100 performs a gesture that is the first action content or after the robot reproduces a voice that is the first action content, the action determination unit 236 determines whether or not the action of the user has been corrected by detecting the action of the user, and in a case in which the action of the user has not been corrected, the action determination unit 236 may cause the action control unit 250 to operate the robot 100 so as to implement a third action content that is different from the first action content.

The case in which the action of the user has not been corrected may be interpreted as a case in which the user has continued dangerous actions and behaviors or a case in which the dangerous situation has not been resolved even though the operation of the robot 100 according to the first action content had been performed.

The third action content may include at least one of transmission of specific information to a person other than the user, execution of a gesture that attracts interests of the user, reproduction of a sound that attracts interests of the user, or reproduction of a video that attracts interests of the user.

The transmission of specific information to a person other than the user may include distribution of an e-mail describing a warning message to a guardian, a nursery-school teacher, or the like of the user, distribution of an image (still image or moving image) including the user and the surrounding scenery, and the like. Furthermore, the transmission of specific information to a person other than the user may include distribution of a voice of a warning message.

The gesture that attracts interests of the user may include a body gesture and a hand gesture of the robot 100. Specifically, the robot 100 may swing both arms widely, blink the LEDs of the eye part of the robot 100, and the like.

The reproduction of a sound that attracts interests of the user may include specific music that the user likes, and may also include a voice saying “come here” or “Let's play together”, or the like.

The reproduction of a video that attracts interests of the user may include an image of an animal raised by the user, an image of the parents of the user, and the like.

According to the robot 100 of the disclosure, in a case in which, in the autonomous processing, whether or not a child or the like is about to perform a dangerous behavior (for example, going up to a window edge to open the window) has been detected and a danger has been sensed, the robot can autonomously perform an action of correcting the action of the user. As a result, the robot 100 can autonomously perform a gesture and make an utterance with the contents such as “Stop”, “[Name], it's dangerous. Come here”, or the like. Furthermore, in a case in which the child stops dangerous behavior after verbal intervention, the robot 100 can also perform an action of praising the child, saying “Are you OK? You listened well”, or the like. In addition, in a case in which the child does not stop the dangerous behavior, the robot 100 can encourage the child to stop the dangerous behavior by sending a warning email to the parent or the nursery school teacher, sharing the situation with a moving image, performing a movement in which the child is interested, playing a moving image in which the child is interested, or playing music in which the child is interested.

For example, multiple types of robot actions include the following (1) to (26).
(1) The robot does nothing.
(2) The robot dreams.
(3) The robot speaks to the user.
(4) The robot creates a picture diary.
(5) The robot proposes an activity.
(6) The robot proposes a person whom the user should meet.
(7) The robot introduces news that the user is interested in.
(8) The robot edits pictures and videos.
(9) The robot studies with the user.
(10) The robot evokes a memory.
(11) The robot 100 can execute a body gesture and a hand gesture to guide the user to a place other than the stairs as the first action content for correcting the action of the user.
(12) The robot 100 can execute a body gesture and a hand gesture and the like to make the user remain in that place as the first action content for correcting the action of the user.
(13) The robot 100 can reproduce a voice for guiding the user to a place other than the stairs as the first action content for correcting the action of the user.
(14) The robot 100 can reproduce a voice and the like to make the user remain in that place as the first action content for correcting the action of the user.
(15) The robot 100 can execute a body gesture and a hand gesture to make the user on the chest of the drawers or about to climb on the chest of the drawers remain in that place, or a body gesture and a hand gesture to move the user to a place other than the current place, as the first action content for correcting the action of the user.
(16) The robot 100 can execute a body gesture and a hand gesture to make the user who is on a window edge or who is on the window edge and putting his/her hands on the window remain in that place, or a body gesture and a hand gesture to move the user to a place other than the current place, as the first action content for correcting the action of the user. (17) The robot 100 can execute a body gesture and a hand gesture to make the user who is walking on a fence or trying to climb on the fence remain in that place, or a body gesture and a hand gesture to move the user to a place other than the current place, as the first action content for correcting the action of the user.
(18) The robot 100 can execute a body gesture and a hand gesture to make the user who is walking on a roadway or having entered a roadway from a sidewalk remain in that place, or a body gesture and a hand gesture to move the user to a place other than the current place, as the first action content for correcting the action of the user.
(19) In a case in which an action of the user has been corrected, the robot 100 can execute reproduction of at least one of a voice praising the action of the user or a voice expressing gratitude for the action of the user, as the second action content different from the first action content.
(20) In a case in which an action of the user has not been corrected, the robot 100 can execute transmission of specific information to a person other than the user, as a third action content different from the first action content.
(21) The robot 100 can execute a gesture that attracts interests of the user as the third action content.
(22) The robot 100 can execute at least one of reproduction of sound that attracts interests of the user or reproduction of a video that attracts interests of the user as the third action content.
(23) As the transmission of specific information to a person other than the user, the robot 100 can distribute an email containing a warning message to a guardian, a nursery school teacher, or the like of the user.
(24) The robot 100 can distribute an image (still image or moving image) containing the user and the surrounding scenery as transmission of specific information to a person other than the user.
(25) As the transmission of specific information to a person other than the user, the robot 100 can distribute a voice of a warning message.
(26) The robot 100 can execute at least one of swinging both arms widely and blinking the LEDs of the eye portion of the robot 100 as a gesture that attracts interest of the user.

In a case in which the action determination unit 236 detects an action of the user spontaneously or periodically and determines to correct the action of the user as an action of the electronic equipment that is a robot action based on the detected action of the user and the specific information stored in advance, the action determination unit 236 can implement the following first action content.

The action determination unit 236 can execute, as the robot action, the first action content of “(11)” described above, in other words, a body gesture and a hand gesture to guide the user to a place other than the stairs.

The action determination unit 236 can execute, as the robot action, the first action content of “(12)” described above, in other words, a body gesture and a hand gesture to make the user remain in that place.

The action determination unit 236 can execute, as the robot action, the first action content of “(13)” described above, in other words, reproduce a voice for guiding the user to a place other than the stairs.

The action determination unit 236 can execute, as the robot action, the first action content of “(14)” described above, in other words, reproduce a voice for making the user remain in that place.

The action determination unit 236 can implement the first action content of “(15)” described above as the robot action. That is, the action determination unit 236 can perform a body gesture and a hand gesture to make the user on the chest of drawers or about to climb on the chest of the drawers remain in that place, or a body gesture and a hand gesture to move the user to a place other than the current place.

The action determination unit 236 can implement the first action content of “(16)” described above as the robot action. That is, the action determination unit 236 can perform a body gesture and a hand gesture to make the user at the window edge or at the window edge and placing his/her hand on the window remain in that place, or a body gesture and a hand gesture to move the user to a place other than the current place.

The action determination unit 236 can implement the first action content of “(17)” described above as the robot action. That is, the action determination unit 236 can perform a body gesture and a hand gesture to make the user walking on a fence or about to climb on the fence remain in that place, or a body gesture and a hand gesture to move the user to a place other than the current place.

The action determination unit 236 can implement the first action content of “(18)” described above as the robot action. That is, the action determination unit 236 can perform a body gesture and a hand gesture to make the user walking on a roadway or having entered the roadway from a sidewalk remain in that place, or a body gesture and a hand gesture to move the user to a place other than the current place.

In a case in which the action of the user has been corrected, the action determination unit 236 can implement the second action content different from the first action content. Specifically, the action determination unit 236 can implement, as the robot action, the second action content of “(19)” described above, in other words, reproduction of at least one of a voice praising the action of the user or a voice expressing gratitude for the action of the user.

In a case in which the action of the user has not been corrected, the action determination unit 236 can execute the third action content different from the first action content. Hereinafter, examples of the third action content will be described.

The action determination unit 236 can execute, as the robot action, the third action content of “(20)” described above, in other words, transmission of specific information to a person other than the user.

The action determination unit 236 can execute, as the robot action, the third action content of “(21)” described above, in other words, a gesture that attracts interests of the user.

The action determination unit 236 can execute, as the robot action, the third action content of “(22)” described above, that is, at least one of reproduction of a sound that attracts interests of the user or reproduction of a video that attracts interests of the user.

The action determination unit 236 can execute, as the robot action, the third action content of “(23)” described above, in other words, distribution of an email containing a warning message to a guardian, a nursery school teacher, or the like of the user, as the transmission of specific information to a person other than the user.

The action determination unit 236 can execute, as the robot action, the third action content of “(24)” described above, that is, distribution of an image (still image or moving image) containing the user and the surrounding scenery as the transmission of specific information to a person other than the user.

The action determination unit 236 can execute, as the robot action, the third action content of “(25)” described above, in other words, distribution of a voice for a warning message, as the transmission of specific information to a person other than the user.

The action determination unit 236 can execute, as the robot action, the third action content of “(26)” described above, in other words, at least one of having the robot 100 swing both arms widely or blinking the LEDs of the eye portion of the robot 100 as a gesture that attracts interests of the user.

Furthermore, in a case in which a voice for guiding the user to a place other than the stairs is reproduced as the first action content indicated in “(13)” described above, the related information collection unit 270 may store voice data for guiding the user to a place other than the stairs in the collected data 223.

Furthermore, in a case in which a voice or the like for making the user remain in that place is reproduced as the first action content indicated in “(14)” described above, the related information collection unit 270 may store voice data for making the user remain in that place in the collected data 223.

Furthermore, in a case in which at least one of a voice praising the action of the user or a voice expressing gratitude for the action of the user is reproduced as the second action content indicated in “(19)” described above, the related information collection unit 270 may store these pieces of voice data in the collected data 223.

In addition, the memory control unit 238 may store the above-described table information in the history data 222. Specifically, the memory control unit 238 may store, in the history data 222, table information that is information in which an answer of the generative AI to a question, a detected action of the user, and an action content for correcting the action are associated with each other.

### (Outline of First Action Content)

In particular, in a case in which the action determination unit 236 detects, as an action of the avatar, an action of the user spontaneously or periodically, and determines, as an action of the avatar, to correct the action of the user based on the detected action of the user and specific information stored in advance, it is preferable for the action determination unit to cause the action control unit 250 to display the avatar in the image display area of the headset-type terminal 820 to execute the first action content.

### (Outline of Second Action Content)

In a case in which, after the avatar is caused to perform a gesture by the action control unit 250 or after the avatar is caused to reproduce a voice by the action control unit 250, the action determination unit 236 determines whether or not the action of the user has been corrected by detecting the action of the user, and in a case in which the action of the user has been corrected, it is preferable for the action determination unit 236 to cause the action control unit 250 to display the avatar in the image display area of the headset-type terminal 820 so as to execute the second action content that is different from the first action content as an action of the avatar.

### (Outline of Third Action Content)

In a case in which, after the avatar is caused to perform a gesture by the action control unit 250 or after the avatar is caused to reproduce a voice by the action control unit 250, the action determination unit 236 detects an action of the user and determines whether or not the action of the user has been corrected, and in a case in which the action of the user has not been corrected, it is preferable for the action determination unit 236 to cause the action control unit 250 to display the avatar in the image display area of the headset-type terminal 820 so as to execute the third action content that is different from the first action content as an action of the avatar.

Hereinafter, the first to third action contents will be specifically described from the first action content.

In the autonomous processing in the embodiment, the action determination unit 236 spontaneously or periodically may detect a state or an action of the user. The term “spontaneously” may be interpreted as meaning that the action determination unit 236 spontaneously acquires a state or an action of the user without a trigger from outside. The trigger from outside may include a question from the user to the avatar, an active action from the user to the avatar, or the like. The term “periodically” may be interpreted as a specific cycle such as in units of one second, one minute, one hour, several hours, several days, week, or day of the week.

The state of the user may include an action tendency of the user. The action tendency may be interpreted as an action tendency of a hyperactive or impulsive user, such as a user frequently running up stairs, a user frequently climbing or trying to climb a dresser, or a user frequently climbing on a window edge to open the window. In addition, the action tendency may be interpreted as a tendency of an action with hyperactivity or impulsiveness, such as a user frequently walking on or trying to walk on a fence, or a user frequently walking on a roadway or entering a roadway from a sidewalk.

Furthermore, in the autonomous processing, the action determination unit 236 may ask a question to the generative AI about the detected state or action of the user, and store an answer of the generative AI to the question and the detected action of the user in association with each other. At this time, the action determination unit 236 may store action contents for correcting the action in association with the answer.

Information in which the answer of the generative AI to the question, the detected action of the user, and the action content for correcting the action are associated with each other may be recorded as table information in a storage medium such as a memory. The table information may be interpreted as specific information recorded in the storage unit.

Furthermore, in the autonomous processing, an action plan of the avatar for calling attention to the state or action of the user may be set based on the detected action of the user and the stored specific information.

As described above, the action determination unit 236 can record table information in which the answer of the generative AI corresponding to the state or action of the user is associated with the detected state or action of the user in the storage medium. Hereinafter, an example of contents stored in the table will be described.

(1. A Case in which the User Tends to Frequently Run on Stairs)

In the case of this tendency, the action determination unit 236 itself asks the generative AI a question “What other things is the child who performs such an action likely to do?”. In a case in which the answer of the generative AI to this question is, for example, “The user is likely to stumble on the stairs”, the action determination unit 236 may store the action of the user running on the stairs and the answer of the generative AI in association with each other. At this time, the action determination unit 236 may store action contents for correcting the action in association with the answer, as an action of the avatar controlled by the action control unit 250.

The action content for correcting the action may include at least one of execution of a gesture of the avatar controlled by the action control unit 250 to correct a dangerous action of the user or reproduction of a voice of the avatar controlled by the action control unit 250 to correct the action of the user.

The gesture for correcting the dangerous action may include a body gesture and a hand gesture to guide the user to a specific place, a body gesture and a hand gesture to make the user remain in that place, and the like. The specific place may include a place other than the place where the user is currently located, for example, the vicinity of the avatar, a space inside a window, or the like.

The voice for correcting the dangerous action may include a voice saying “Stop” or “[Name], it's dangerous, so don't move”, or the like. The voice for correcting the dangerous action may include a voice saying “Do not run” or “stay still”, or the like.

(2. A Case in which the User Tends to Frequently Stay on or Try to Climb a Chest of Drawers)

In a case in which the user has such a tendency, the action determination unit 236 asks the generative AI a question in the same manner as described above. In a case in which an answer of the generative AI to the question is, for example, “The user may fall from the chest of drawers” or “The user may be caught in the door of the chest of drawers”, the action determination unit 236 may store the action of the user who is on the chest of drawers or who is trying to climb on the chest of drawers in association with the answer of the generative AI. In addition, the action determination unit 236 may store action contents for correcting the action in association with the answer, as an action of the avatar.

(3. A Case in which the User Frequently Tends to Climb on a Window Edge to Open the Window)

In a case in which the user has such a tendency, the action determination unit 236 asks the generative AI a question in the same manner as described above. In a case in which an answer of the generative AI to the question is, for example, “The user may put his/her face out of the window” or “The user may be caught in the window”, the action determination unit 236 may store the action of the user who is climbing on the window edge to open the window in association with the answer of the generative AI. In addition, the action determination unit 236 may store action contents for correcting the action in association with the answer, as an action of the avatar.

(4. A Case in which the User Frequently Walks on or Tries to Climb on the Fence)

In a case in which the user has such a tendency, the action determination unit 236 asks the generative AI a question in the same manner as described above. In a case in which an answer of the generative AI to the question is, for example, “The user may fall from the fence” or “The user may be hurt by the unevenness of the wall”, the action determination unit 236 may store the action of the user who is walking on or trying to climb on the fence in association with the answer of the generative AI. In addition, the action determination unit 236 may store action contents for correcting the action in association with the answer, as an action of the avatar.

(5. A Case in which the User Frequently Walks on a Roadway or Enters a Roadway from a Sidewalk)

In a case in which the user has such a tendency, the action determination unit 236 asks the generative AI a question in the same manner as described above. In a case in which an answer of the generative AI to the question is, for example, “You may cause a traffic accident” or “You may cause a traffic jam”, the action determination unit 236 may store the action of the user who is walking on a roadway or has entered a roadway from a sidewalk in association with the answer of the generative AI. In addition, the action determination unit 236 may store action contents for correcting the action in association with the answer, as an action of the avatar.

As described above, in the autonomous processing, a table in which the answer of the generative AI corresponding to the state or action of the user, the content of the state or action, and the action content for correcting the state or action as an action of the avatar are associated with each other may be recorded in a storage medium such as a memory.

Furthermore, in the autonomous processing, after the table is recorded, the action of the user is autonomously or periodically detected, and an action plan of the avatar that urges the user to pay attention may be set based on the detected action of the user and the content of the stored table. Specifically, the action determination unit 236 of the avatar may cause the action control unit 250 to operate the avatar so as to execute the first action content for correcting the action of the user based on the detected action of the user and the content of the stored table. Hereinafter, an example of the first action content will be described.

(1. A Case in which the User Tends to Frequently Run on Stairs)

In a case in which the user running on the stairs is detected, the action determination unit 236 may cause the action control unit 250 to operate the avatar such that the avatar performs a body gesture and a hand gesture to guide the user to a place other than the stairs, a body gesture and a hand gesture to make the user remain in that place, and the like as the first action content for correcting the action. The action control unit 250 may transform the avatar in human form into a symbol for guiding the user to a place other than the stairs (for example, an arrow mark indicating a direction), a symbol for making the user remain in that place (for example, a “STOP” mark), or the like, instead of a body gesture and a hand gesture, and display the symbol in the image display area of the headset-type terminal 820.

Furthermore, the action determination unit 236 may cause the action control unit 250 to operate the avatar so as to reproduce, as the first action content for correcting the action, a voice of the avatar to guide the user to a place other than the stairs, a voice of the avatar to make the user remain still in that place, or the like. The voice may include “[Name], it's dangerous, so don't run”, “Don't move”, “Don't run”, “Stay still”, or the like. Together with these voices, the action control unit 250 may display callout comments such as “[Name], it's dangerous. Do not run”, “Don't move”, or the like near the mouth of the avatar in human form in the image display area of the headset-type terminal 820.

(2. A Case in which the User Tends to Frequently Stay on or Try to Climb a Chest of Drawers)

The action determination unit 236 may cause the action control unit 250 to operate the avatar such that the avatar performs a body gesture and a hand gesture to make the user who is on the chest of the drawers or about to climb on the chest of the drawers remain still in that place, or a body gesture and a hand gesture to move the user to a place other than the current place. The action control unit 250 may transform the avatar in human form into a symbol (for example, a “STOP” mark) for making the user remain still in that place, an animation of the avatar to move the user to a place other than the current place (for example, an arrow mark extending so as to indicate a direction and a distance), or the like, instead of a body gesture and a hand gesture of the avatar, and display the transformed avatar in the image display area of the headset-type terminal 820.

(3. A Case in which the User Frequently Tends to Climb on a Window Edge to Open the Window)

The action determination unit 236 may cause the action control unit 250 to operate the avatar such that, for the user who is on the window edge or at the window edge and putting his/her hand at the window, the avatar performs a body gesture and a hand gesture to make the user remain still in that place, or a body gesture and a hand gesture to move the user to a place other than the current place. The action control unit 250 may transform the avatar in human form into a symbol (for example, a “STOP” mark) for making the user remain still in that place, an animation of the avatar to move the user to a place other than the current place (for example, an arrow mark extending so as to indicate a direction and a distance), or the like, instead of a body gesture and a hand gesture of the avatar, and display the transformed avatar in the image display area of the headset-type terminal 820.

(4. A Case in which the User Frequently Walks on or Tries to Climb on the Fence)

The action determination unit 236 may cause the action control unit 250 to operate the avatar such that the avatar performs a body gesture and a hand gesture to make the user who is walking on a fence or trying to climb on the fence remain still in that place, or a body gesture and a hand gesture to move the user to a place other than the current place. The action control unit 250 may transform the avatar in human form into a symbol (for example, a “STOP” mark) for making the user remain still in that place, an animation of the avatar to move the user to a place other than the current place (for example, an arrow mark extending so as to indicate a direction and a distance), or the like, instead of a body gesture and a hand gesture of the avatar, and display the transformed avatar in the image display area of the headset-type terminal 820.

(5. A Case in which the User Frequently Walks on a Roadway or Enters a Roadway from a Sidewalk)

The action determination unit 236 may cause the action control unit 250 to operate the avatar such that the avatar performs a body gesture and a hand gesture to make the user who is walking on a roadway or who has entered the roadway from a sidewalk remain still in that place, or a body gesture and a hand gesture to move the user to a place other than the current place. The action control unit 250 may transform the avatar in human form into a symbol (for example, a “STOP” mark) for making the user remain still in that place, an animation of the avatar to move the user to a place other than the current place (for example, an arrow mark extending so as to indicate a direction and a distance), or the like, instead of a body gesture and a hand gesture of the avatar, and display the transformed avatar in the image display area of the headset-type terminal 820.

In a case in which, after the avatar performs a gesture that is the first action content or after the avatar reproduces a voice that is the first action content, the action determination unit 236 determines whether or not the action of the user has been corrected by detecting the action of the user, and in a case in which the action of the user has been corrected, the action determination unit 236 may cause the action control unit 250 to operate the avatar so as to execute, as an action of the avatar, a second action content that is different from the first action content.

The case in which the action of the user has been corrected may be interpreted as a case in which, as a result of execution of the operation of the avatar according to the first action content, the user has stopped dangerous actions and behaviors or the dangerous situation for the user has been resolved.

The second action content may include reproduction of at least one of a voice of the avatar, controlled by the action control unit 250, praising the action of the user and a voice of the avatar expressing gratitude for the action of the user.

The voice praising the action of the user may include a voice indicating “Are you OK? Well listened”, “Good work, great”, or the like. The voice expressing gratitude for the action of the user may include a voice saying “Thank you for coming”. Together with these voices, the action control unit 250 may display callout comments such as “OK? You listened well”, “Good job, great”, or the like near the mouth of the avatar in human form in the image display area of the headset-type terminal 820.

In a case in which, after the avatar performs a gesture that is the first action content or after the avatar reproduces a voice that is the first action content, the action determination unit 236 determines whether or not the action of the user has been corrected by detecting the action of the user, and in a case in which the action of the user has not been corrected, the action determination unit 236 may cause the action control unit 250 to operate the avatar so as to execute the third action content that is different from the first action content as an action of the avatar.

The case in which the action of the user has not been corrected may be interpreted as a case in which the user has continued dangerous actions and behaviors or a case in which the dangerous situation has not been resolved even though the operation of the avatar according to the first action content had been performed.

The third action content may include at least one of transmission of specific information to a person other than the user, or execution of a gesture by the avatar controlled by the action control unit 250 that attracts interests of the user, reproduction of a sound that attracts interests of the user, or reproduction of a video that attracts interests of the user.

The transmission of specific information to a person other than the user may include distribution of an e-mail describing a warning message to a guardian, a nursery-school teacher, or the like of the user, distribution of an image (still image or moving image) including the user and the surrounding scenery, and the like. Furthermore, the transmission of specific information to a person other than the user may include distribution of a voice of a warning message.

A gesture performed by the avatar that attracts interests of the user may include body gestures and hand gestures of the avatar controlled by the action control unit 250. Specifically, the gestures may include, under control of the action control unit 250, widely swinging both arms of the avatar, blinking the LEDs of the eye portion of the avatar, or the like. The action control unit 250 may attract interests of the user by transforming the avatar in human form into an animal form, a character featured in a popular animation, a popular local mascot, or the like, instead of the body gesture and hand gesture of the avatar.

The reproduction of a sound that attracts interests of the user may include specific music that the user likes, and may also include a voice saying “come here” or “Let's play together”, or the like.

The reproduction of a video that attracts interests of the user may include an image of an animal raised by the user, an image of the parents of the user, and the like.

According to the disclosure, in a case in which, in the autonomous processing, whether or not a child or the like is about to perform a dangerous behavior (going up to a window edge to open the window) has been detected and a danger has been sensed, an action of correcting the action of the user can be autonomously performed. As a result, the avatar controlled by the action control unit 250 can autonomously perform a gesture and make an utterance with the contents such as “Stop”, “[Name], it's dangerous. Come here”, or the like. Furthermore, in a case in which the child stops dangerous behavior after verbal intervention, the avatar controlled by the action control unit 250 can also perform an action of praising the child, saying “Are you OK? You listened well”, or the like. In addition, in a case in which the child does not stop the dangerous behavior, the avatar controlled by the action control unit 250 can encourage the child to stop the dangerous behavior by sending a warning email to the parent or the nursery school teacher, sharing the situation with a moving image, performing a movement in which the child is interested, playing a moving image in which the child is interested, or playing music in which the child is interested.

### Thirteenth Embodiment

In the autonomous processing in the embodiment, the robot 100 as an agent spontaneously and periodically detects states of the user. More specifically, the robot 100 spontaneously and periodically detects whether the user and his/her family use a social networking service (hereinafter, referred to as social media). That is, the robot 100 constantly monitors a display of a smartphone or the like owned by each of the user and his/her family member and detects social media use states. In a case in which the user is a child, the robot 100 spontaneously considers a way of engaging with the social media and a post content while conversing with the child.

For example, multiple types of the robot actions include the following (1) to (11).
(1) The robot does nothing.
(2) The robot dreams.
(3) The robot speaks to the user.
(4) The robot creates a picture diary.
(5) The robot proposes an activity.
(6) The robot proposes a person whom the user should meet.
(7) The robot introduces news that the user is interested in.
(8) The robot edits pictures and videos.
(9) The robot studies with the user.
(10) THE ROBOT EVOKES A MEMORY.
(11) THE ROBOT GIVES ADVICE ON THE SOCIAL MEDIA TO THE USER.

In a case in which the action determination unit 236 determines that, as a robot action, “(11) The robot gives advice on the social media to the user.”, in other words, advice on the social media is given to the user, the robot 100 determines the utterance content of the robot corresponding to the information stored in the collected data 223 using the sentence generation model. At this time, the action control unit 250 causes a speaker included in the control target 252 to output a voice representing the determined utterance content of the robot. Note that, in a case in which the user 10 is absent around the robot 100, the action control unit 250 stores the determined utterance content of the robot in the action plan data 224 without outputting a voice representing the determined utterance content of the robot.

Specifically, the robot 100 proposes a way of engaging with the social media and a post content for social media for the user to appropriately use social media with reassurance and safety, while conversing with the user. For example, the robot 100 proposes a combination of one or more of information security measures, protection of personal information, prohibition of defamation, prohibition of spread of false information, and compliance with laws to the user as a way of engaging with social media. As a specific example, for the question “What should be noted when using social media?” in the conversation with the user, the robot 100 can propose a way of engaging with the social media, providing advice “It is better to be careful not to disclose personal information on the Internet!”.

On the other hand, the robot 100 proposes a post content satisfying a predetermined condition including a combination of one or more of information security measures, protection of personal information, prohibition of slander or defamation, prohibition of spread of false information, and compliance with laws to the user. As a specific example, with respect to an utterance “I want to post something that would not cause a controversy between A and B” in a conversation with the user, the robot 100 can think about a post content that would not slander or defame both parties such as “Both A and B are great!” and propose the content to the user.

Furthermore, in a case in which the robot 100 recognizes the user as a minor, the robot makes a proposal about one or both of the way of engaging with social media and a post content for social media for minors while conversing with the user. Specifically, the robot 100 can make a proposal about the way of engaging with the social media and the post content for the social media under stricter conditions applied to minors. As a specific example, for the question “What should be noted when using social media?” in a conversation with the user who is a minor, the robot 100 can propose a way of engaging with the social media, providing advice “It is better to be careful not to publicly disclose personal information, engage in slander or defamation, or spread rumors (false information)”. Further, with respect to an utterance “I want to post something that would not cause a controversy between A and B” in a conversation with the user who is a minor, the robot 100 can think about the content of a polite expression in a post that would not slander or defame both parties such as “Both A and B are great!” and propose the content to the user.

Furthermore, the robot 100 can make an utterance related to posting when the user finishes posting to the social media regarding the post content on the social media as an action of making proposal related to the use of the social media. For example, the robot 100 can spontaneously utter a content such as “In this post, you are fully conscious of the way of engaging with the social media, so you get 100 points!” after the user finishes posting to the social media.

Furthermore, the robot 100 can analyze the post content posted by the user, and make a proposal on the way of engaging with the social media or a way of creating a post content to the user based on the analysis result. For example, in a case in which there is no utterance from the user, the robot 100 can perform an utterance with contents such as “This post content contains contents different from the fact and may be a rumor (false information), so be careful!” based on the post content of the user.

Furthermore, the robot 100 proposes, to the user, one or both of the way of engaging with the social media or the post content on the social media in a conversation form based on a state or action of the user. For example, in a case in which the user holds a terminal device in his/her hand and the robot 100 recognizes that “The user may be in trouble with how to use the social media.”, the robot 100 can talk to the user in a conversation form and propose a method of using the social media, a way of engaging with the social media, and a post content.

In addition, regarding “(11) The robot gives advice on the social media to the user.”, the related information collection unit 270 acquires information regarding the social media in advance. For example, the related information collection unit 270 may periodically access an information source such as a television, the web, or the like by itself, voluntarily collect information regarding laws and regulations, incidents, problems, or the like related to the social media, and store the collected information in the collected data 233. As a result, since the robot 100 can acquire the latest information regarding the social media, it is possible to voluntarily give advice corresponding to the latest problems and the like regarding the social media to the user.

Based on the state of the user 10 recognized by the state recognition unit 230, in a case in which an action of the user 10 with respect to the robot 100 has been detected in a state where there is no action of the user 10 with respect to the robot 100, the action determination unit 236 reads data stored in the action plan data 224 and determines an action of the robot 100.

In particular, similarly to the first embodiment, in a case in which the action determination unit 236 determines to give advice on the social media to the user as an action of the avatar, it is preferable to cause the action control unit 250 to control the avatar to give advice on the social media to the user by using the output of the action determination model 221.

Here, the avatar is, for example, a 3D avatar, and may be selected by the user from avatars prepared in advance, may be a virtual avatar of the user, or may be a favorite avatar generated by the user. To generate an avatar, image generative AI may be utilized to generate an avatar in multiple art styles such as photorealistic, cartoon, moe-style, and oil painting style.

Furthermore, as in the first embodiment, in a case in which the action determination unit 236 determines, as an action of the avatar, to give advice on social media to the user by using the output of the action determination model 221, the action determination unit 236 may cause the action control unit 250 to perform control such that at least one of the type, voice, or expression of the avatar is changed according to the user who is a recipient of the advice. The avatar may imitate a real person, an imaginary person, or a character. Specifically, the type of the avatar that gives advice on the social media may be a parent, an elder brother or an elder sister, a school teacher, a celebrity, or the like. However, in a case in which the user who is the recipient of the advice is a minor or a child, the action control unit 250 may be caused to perform control such that the avatar is transformed into an avatar that persuades the user more gently, an avatar with a gentler voice, or an avatar that speaks with a gentle smiling expression, such as a grandmother, a gentle elder sister, or a user's favorite character. In addition, as in the first embodiment, in a case in which the action determination unit 236 determines, as an action of the avatar, to give advice on the social media to the user by using an output of the action determination model 221, the action determination unit may cause the action control unit 250 to control the avatar to transform into an animal different from a person, for example, a dog, a cat, or the like.

### Fourteenth Embodiment

In the present embodiment, a user 10a, a user 10b, a user 10c, and a user 10d constitute a family as an example. In other words, the user 10a, the user 10b, the user 10c, and the user 10d are members of a family. Furthermore, the users 10a to 10d may include a caregiver who provides care. For example, in a case in which the user 10a is a caregiver, the user may provide care to a person (user) other than the family members, or provide care to the user 10b who is a family member. As an example, the user 10a is a caregiver, and the user 10b is a care receiver who receives care.

Note that, as will be described later, the robot 100 provides the user 10 with advice information regarding care, but in a case in which the user 10a who is a caregiver provides care for a person other than the family members, the user 10 in this case may not be a person constituting the family. In a case in which the user 10b who is a care receiver receives care from a person (user) other than the family members, the user 10 in this case may not be a person constituting the family. Furthermore, as will be described later, the robot 100 provides the user 10 with advice information regarding health of the family or advice information regarding the mental states, but the user 10 in this case may not include a caregiver or a care receiver.

The robot 100 according to the embodiment can provide advice information regarding caregiving. Although the robot 100 provides the advice information regarding caregiving to the user 10 including the caregiver and the care receiver, the embodiment is not limited thereto, and the robot may provide the advice information to any user such as a family member including at least one of the caregiver or the care receiver, for example.

Specifically, the robot 100 recognizes mental and physical states of the user 10 including at least one of the caregiver or the care receiver. Here, the mental and physical states of the user 10 include, for example, the degree of stress, the degree of fatigue of the user 10, and the like. The robot 100 provides advice information regarding caregiving according to the recognized mental and physical states of the user 10.

As an example, in a case in which the degree of stress of the user 10 is estimated to be relatively high or the degree of fatigue of the user 10 is estimated to be relatively high based on the action of the user or the like, the robot 100 executes an action of starting a conversation with the user 10. Specifically, the robot 100 makes an utterance indicating that advice information such as “I have advice about caregiving” is to be provided.

Subsequently, the robot 100 generates advice information regarding caregiving based on the recognized mental and physical states of the user 10 (here, the degree of stress, the degree of fatigue, and the like). The advice information includes information regarding recovery of the mind and body of the user 10, such as a method of maintaining motivation for caregiving, a method of relieving stress, and a relaxation method, but is not limited thereto. Here, the robot 100 makes an utterance and provides advice information according to the mental and physical states of the user 10, for example, “You seem to be very stressed (fatigued). I recommend moving your body with stretches” and the like.

As described above, in the embodiment, the robot 100 recognizes the mental and physical states of the user 10 including the caregiver or the like, and executes an action corresponding to the recognized mental and physical states, thereby being able to give appropriate advice on caregiving to the user 10. In other words, the robot 100 can understand the stress and fatigue of the user 10 and provide appropriate advice information such as a relaxation method and a stress relief method. That is, the robot 100 according to the embodiment can perform an action appropriate for the user 10.

Furthermore, in a case in which the control unit of the robot 100 recognizes mental and physical states of the user 10 including at least one of the caregiver and the care receiver, the control unit determines an action of providing advice information regarding caregiving according to the recognized state as its own action. As a result, the robot 100 can provide appropriate advice information regarding caregiving in accordance with the mental and physical states of the user 10 including the caregiver and the care receiver.

Furthermore, in a case in which at least one of the degree of stress and the degree of fatigue of the user 10 is recognized as the mental and physical states of the user 10, the control unit of the robot 100 generates information regarding mental and physical recovery of the user 10 as the advice information based on at least one of the recognized degree of stress and degree of fatigue. As a result, the robot 100 can provide, as the advice information, the information regarding the mental and physical recovery of the user 10 according to the degree of stress and the degree of fatigue of the user 10.

The storage unit 220 includes history data 222. The history data 222 includes a history of past emotion values and actions of the user 10. The emotion value and the action history are recorded for each user 10 by being associated with identification information of the user 10, for example. Furthermore, the history data 222 may include user information of each of the plurality of users 10 associated with the identification information of the user 10. The user information includes information indicating that the user 10 is a caregiver, information indicating that the user 10 is a care receiver, and information indicating that the user is neither a caregiver nor a care receiver. The user information indicating whether the user 10 is a caregiver or the like may be estimated from the action history of the user 10 or may be registered by the user 10 himself/herself. Furthermore, the user information includes information indicating characteristics of the user 10 such as personality, interests, and inclinations of the user 10. The user information indicating characteristics of the user 10 may be estimated from the action history of the user 10 or may be registered by the user 10 himself/herself. At least a part of the storage unit 220 is implemented by a storage medium such as a memory. A person DB that stores face images of the user 10, attribute information of the user 10, and the like may be included.

The state recognition unit 230 recognizes the mental and physical states of the user 10 based on the information analyzed by the sensor module unit 210. For example, in a case in which the recognized user 10 is determined to be a caregiver or a care receiver based on the user information, the state recognition unit 230 recognizes the mental and physical states of the user 10. Specifically, the state recognition unit 230 estimates the degree of stress of the user 10 based on various types of information such as text information indicating an action, an expression, a voice, and an utterance content of the user 10, and recognizes the estimated degree of stress as mental and physical states of the user 10. As an example, in a case in which information indicating that stress is involved is included in the various types of information (a feature value such as a frequency component of a voice, text information, and the like), the user state recognition unit 230 estimates that the degree of stress of the user 10 is relatively high. Specifically, the user state recognition unit 230 estimates the degree of fatigue of the user 10 based on various types of information such as text information indicating an action, an expression, a voice, and an utterance content of the user 10, and recognizes the estimated degree of fatigue as mental and physical states of the user 10. As an example, in a case in which information indicating that fatigue has been accumulated is included in the various types of information (a feature value such as a frequency component of a voice, text information, and the like), the user state recognition unit 230 estimates that the degree of fatigue of the user 10 is relatively high. Note that the degree of stress, the degree of fatigue, and the like described above may be registered by the user 10 himself/herself.

Note that the state recognition unit 230 may recognize both the degree of stress and the degree of fatigue, or may recognize either of them. That is, the state recognition unit 230 may recognize at least one of the degree of stress or the degree of fatigue.

In addition, the state recognition unit 230 recognizes mental and physical states of each of a plurality of users 10 constituting a family based on information analyzed by the sensor module unit 210 or the like. Specifically, the state recognition unit 230 estimates the health states of the user 10 based on various types of information such as text information indicating an action, an expression, a voice, and an utterance content of the user 10, and recognizes the estimated health states as mental and physical states of the user 10. As an example, in a case in which information indicating that the health states are good is included in various types of information (text information or the like), the state recognition unit 230 estimates that the health states of the user 10 are good, and in a case in which information indicating that the health states are poor is included, the state recognition unit estimates that the health states of the user 10 are poor. Specifically, the user state recognition unit 230 estimates lifestyle habits of the user 10 based on various types of information such as text information indicating an action, an expression, a voice, and an utterance content of the user 10, and recognizes the estimated lifestyle habits as mental and physical states of the user 10. As an example, in a case in which information indicating a lifestyle (meal content, exercise habit, or the like) is included in various types of information (text information or the like), the state recognition unit 230 estimates lifestyle habits of the user 10 from such information. Note that the health states, lifestyle habits, and the like described above may be registered by the user 10 himself/herself.

Note that the state recognition unit 230 may recognize both the health states and lifestyle habits, or may recognize either of them. That is, the state recognition unit 230 may recognize at least one of the health states or lifestyle habits.

In addition, the state recognition unit 230 recognizes a mental state of each of a plurality of users 10 constituting a family, as a mental and physical state of the user 10 based on information analyzed by the sensor module unit 210, or the like. Specifically, the state recognition unit 230 estimates the mental states of the user 10 based on various types of information such as text information indicating an action, an expression, a voice, and an utterance content of the user 10, and recognizes the estimated mental states as mental and physical states of the user 10. As an example, in a case in which information indicating a mental state such as being depressed or nervous is included in various types of information (a feature amount such as a frequency component of a voice, text information, or the like), the state recognition unit 230 estimates the mental state of the user 10 from such information. Note that the mental state, and the like described above may be registered by the user 10 himself/herself.

Furthermore, for example, in reaction rules, an action of the robot 100 corresponding to an action pattern in a case in which mental and physical states of the user 10 including a caregiver and a care receiver (a degree of stress and a degree of fatigue) is a state requiring advice on caregiving to the user 10, or a case in which there is a reaction from the user 10 to the provided advice information is determined. For example, in a case in which the degree of stress of the user 10 including the caregiver and the care receiver is estimated to be relatively high or in a case in which the degree of fatigue is estimated to be relatively high based on the reaction rules, the action determination unit 236 determines an action of providing the advice information regarding caregiving corresponding to the mental and physical states of the user 10 to the user 10 as its own action.

In a case in which the action control unit 250 recognizes the mental and physical states of the user 10 including the caregiver and the care receiver, the action control unit determines an action of providing advice information regarding caregiving according to the mental and physical states of the user 10 as its own action, and controls the control target 252.

Specifically, in a case in which the degree of stress of the user 10 is estimated to be relatively high or in a case in which the degree of fatigue is estimated to be relatively high, the action control unit 250 executes an action of starting a conversation with the user 10. Specifically, the action control unit 250 makes an utterance indicating that advice information such as “I have advice about care” is to be provided.

Next, the action control unit 250 generates advice information regarding caregiving based on the recognized mental and physical states of the user 10 (the degree of stress, the degree of fatigue, and the like), and utters and provides the generated advice information. The advice information includes, but is not limited to, information regarding mental and physical recovery of the user 10 by providing mental support to the user 10 (specifically, information for achieving mental and physical recovery), such as a method of maintaining motivation for care, a method of releasing stress, and a relaxation method. For example, the action control unit 250 makes an utterance and provides advice information according to the mental and physical states of the user 10, for example, “You seem to be very stressed. I recommend moving your body with stretches”, “You seem to be very tired. I recommend getting enough sleep”, or the like.

As described above, according to the embodiment, the action control unit 250 recognizes the mental and physical states of the user 10 including the caregiver or the like, and executes an action corresponding to the recognized mental and physical states, thereby being able to give appropriate advice on caregiving to the user 10. In other words, the action control unit 250 can understand the stress and fatigue of the user 10 and provide appropriate advice information such as a relaxation method and a stress relief method.

Furthermore, the action control unit 250 may provide information regarding a law or a system related to caregiving as the advice information. Note that the information regarding the law and system related to caregiving is information corresponding to a caregiving state (care level) of a care receiver, and is acquired from an external server (not illustrated) or the server 300 via the communication network 20 such as the Internet network by the communication processing unit 280, for example, but is not limited thereto.

Furthermore, since the emotion value of the robot 100 is determined by the emotion determination unit 232, the action control unit 250 may utter and provide advice information having contents close to the feeling (emotion) of the user 10a who is a caregiver, for example, “Although caregiving is difficult, the user 10b seems to be greatly helped (he/she seems to be happy)” based on the emotion value or the like.

In the autonomous processing in the embodiment, the robot 100 as an agent spontaneously and periodically detects states of the user 10 who provides care. For example, the robot 100 constantly detects people who provide care, and constantly detects the fatigue level and the sense of well-being of the people who provide care. When determining that the fatigue level or motivation of the user 10 has been lowered, the robot 100 takes an action that enhances motivation or relieves stress. Specifically, the robot 100 understands the stress and fatigue of the user 10, and proposes an appropriate relaxation method and stress relief measure to the user 10. In a case in which the degree of well-being of the caregiver has increased, the robot 100 spontaneously praises the caregiver or gives words of appreciation to the caregiver. In addition, the robot 100 spontaneously and periodically collects information regarding laws and systems regarding caregiving from external data (web sites such as news sites and moving image sites, distribution news, and the like), for example, and in a case in which the degree of importance exceeds a certain value, the robot provides the information collected regarding caregiving to a person (user) who voluntarily provides care.

For example, multiple types of the robot actions include the following (1) to (11).
(1) The robot does nothing.
(2) The robot dreams.
(3) The robot speaks to the user.
(4) The robot creates a picture diary.
(5) The robot proposes an activity.
(6) The robot proposes a person whom the user should meet.
(7) The robot introduces news that the user is interested in.
(8) The robot edits pictures and videos.
(9) The robot studies with the user.
(10) The robot evokes a memory.
(11) The robot gives advice on caregiving to the user.

In a case in which the action determination unit 236 determines that, as a robot action, “(11) Gives advice on caregiving to the user.”, that is, to give advice on necessary information to the user involved in caregiving, for example, the action determination unit acquires necessary information for the user from external data. The robot 100 autonomously acquires such information at all times even when the user is absent.

Furthermore, regarding “Give advice on caregiving to the user.”, for example, the related information collection unit 270 collects information regarding caregiving of the user as the user's preference information and stores the collected information in the collected data 223. Then, this information is output as audio from the speaker or displayed in text on the display, thereby supporting the user's caregiving activities.

In the autonomous processing in the embodiment, the robot 100 as an agent spontaneously and periodically detects states of the user who provides care. For example, the robot 100 constantly detects people who provide care, and constantly detects the fatigue level and the sense of well-being of the people who provide care. When determining that the fatigue level or motivation of the user 10 has been lowered, the robot 100 takes an action that enhances motivation or relieves stress. Specifically, the robot 100 understands the stress and fatigue of the user 10, and proposes an appropriate relaxation method and stress relief measure to the user 10. In a case in which the degree of well-being of the caregiver has increased, the robot 100 spontaneously praises the caregiver or gives words of appreciation to the caregiver. In addition, the robot 100 spontaneously and periodically collects information regarding laws and systems regarding caregiving from external data (web sites such as news sites and moving image sites, distribution news, and the like), for example, and in a case in which the degree of importance exceeds a certain value, the robot 100 spontaneously provides the information collected regarding caregiving to a person (user) who provides care.

The appearance of the robot 100 may imitate an appearance of a person or may be a stuffed toy. Since the robot 100 has a stuffed toy as an external appearance, it is considered that the robot tends to be particularly familiar to children.

In addition, in step S100, the state recognition unit 230 recognizes the state of the user 10 and the state of the robot 100 based on the information analyzed by the sensor module unit 210. For example, in a case in which the recognized user 10 is a caregiver or a care receiver, the state recognition unit 230 recognizes the mental and physical states of the user 10 (the degree of stress, the degree of fatigue, and the like). In addition, the state recognition unit 230 recognizes mental and physical states of a plurality of users 10 constituting a family (health state, lifestyle habits, and the like). Furthermore, the state recognition unit 230 recognizes the mental state of each of the plurality of users 10 constituting the family.

In addition, the action control unit 250 displays the avatar in the image display area of the headset-type terminal 820 as the control target 252C according to the determined action of the avatar. Furthermore, in a case in which the determined action of the avatar includes the utterance content of the avatar, the utterance content of the avatar is output from the speaker as the control target 252C by voice.

In particular, in a case in which the action determination unit 236 determines to give advice on caregiving to the user as an action of the avatar, it is preferable to cause the action control unit 250 to control the avatar to collect information regarding caregiving of the user and give advice on caregiving of the user based on the collected information.

Furthermore, in a case in which the action determination unit 236 determines to give advice on caregiving to the user as an action of the avatar and the advice on caregiving is a caregiving technique using a body, the action determination unit may operate the avatar to demonstrate the caregiving technique. For example, the avatar may be caused to demonstrate a technique that enables easy lifting of the care receiver from the wheelchair to the bed.

Furthermore, in a case in which the action determination unit 236 determines to give advice on caregiving to the user as an action of the avatar, it is preferable to include an action to show appreciation to the user. In this case, the action determination unit 236 may take an action according to the emotion value of the user determined by the emotion determination unit 232. For example, if the emotion value of the user is a disagreeable emotion such as “anxious”, “sad”, “worried”, or the like, an action of making an utterance “It is hard, but you are doing well. Everyone is grateful to you” is taken together with a smile. Furthermore, for example, if the emotion value of the user is a positive emotion such as “joy”, “comfort”, or “a sense of fulfillment”, an action of making an utterance “You are doing your best. “Thank you very much.” is taken together with a smile.

Furthermore, in a case of advising on a method of relieving stress, a relaxation method, or the like, the avatar may be operated so that the avatar is transformed into another avatar, for example, an avatar that moves the body together with the user, such as a yoga instructor or a relaxation instructor. Then, a method for relieving stress, a relaxation method, and the like may be provided through demonstration by the avatar.

### Fifteenth Embodiment

In the autonomous processing in the embodiment, the robot 100 as an agent spontaneously and periodically detects states of the user. The robot 100 constantly monitors contents of conversations of the user on the phone, with friends, or in a company, and detects whether or not the user is suffering from “bullying”, “crime”, “harassment”, or the like. In other words, the robot 100 constantly monitors the contents of conversations of the user on the phone, with friends, or in a company, and detects a risk approaching the user. The robot 100 causes the sentence generation model such as the generative AI to determine whether or not the conversation is with a high probability of bullying, a crime, or the like, and the robot 100 spontaneously contacts, sends an e-mail, or the like to a notification destination registered in advance in a case in which a conversation suspected of the occurrence of the subject matter occurs from the content of the acquired conversation. In addition, the robot 100 describes and contacts about a case assumed from a conversation log of the corresponding part, a probability of occurrence, and a proposal for a solution. The robot 100 can brush up the accuracy of the detection of the event and the proposal for the solution by feeding back the occurrence of the event, the solution status, and the like.

For example, multiple types of the robot actions include the following (1) to (11).
(1) The robot does nothing.
(2) The robot dreams.
(3) The robot speaks to the user.
(4) The robot creates a picture diary.
(5) The robot proposes an activity.
(6) The robot proposes a person whom the user should meet.
(7) The robot introduces news that the user is interested in.
(8) The robot edits pictures and videos.
(9) The robot studies with the user.
(10) The robot evokes a memory.
(11) Advice on risks such as “bullying”, “crime”, and “harassment” is given to the user.

In a case in which the action determination unit 236 determines that, as the robot action, “(11) Advice on risks such as “bullying”, “crime”, and “harassment” is given to the user.”, that is, to give advice on risks such as “bullying”, “crime”, “harassment”, and the like to the user, the robot 100 acquires conversation contents of a plurality of users 10. Specifically, the speech understanding unit 212 analyzes voices of the plurality of users 10 detected by the microphone 201, and outputs text information indicating conversation contents of the plurality of users 10. Furthermore, the robot 100 acquires emotion values of the plurality of users 10. Specifically, voices of the plurality of users 10 and videos of the plurality of users 10 are acquired, and emotion values of the plurality of users 10 are acquired. Furthermore, the robot 100 determines whether or not a specific case such as “bullying”, “crime”, or “harassment” has occurred based on the conversation contents of the plurality of users 10 and the emotion values of the plurality of users 10. Specifically, the action determination unit 236 compares data of a specific case such as past “bullying”, “crime”, and “harassment” stored in the storage unit 220 with conversation contents of the plurality of users 10, thereby determining the similarity between the conversation contents and the specific case. Note that the action determination unit 236 may determine whether the conversation has a high probability of bullying, a crime, or the like by causing a sentence generation model such as generative AI to read sentences of the conversation. Then, the action determination unit 236 determines the possibility that a specific case has occurred based on the similarity between the conversation contents and the specific case and the emotion values of the plurality of users 10. As an example, in a case in which the similarity between the conversation contents and the specific case is high and the emotion values of “anger”, “sorrow”, “discomfort”, “anxiety”, “sadness”, “worry”, and “sense of emptiness” of the plurality of users 10 are high, the action determination unit 236 determines the possibility that the specific case has occurred as a high value. Furthermore, the robot 100 determines an action according to the possibility that the specific case has occurred.

Specifically, in a case in which the possibility that the specific case has occurred exceeds a predetermined threshold value, the action determination unit 236 determines an action for delivering the fact that the possibility that the specific case has occurred is high. For example, the action determination unit 236 may determine to notify by e-mail, the manager of the organization to which the plurality of users 10 belong of the fact that there is a high possibility that the specific case has occurred. Then, the robot 100 executes the determined action. As an example, the robot 100 transmits the above mail to the manager of the organization to which the users 10 belong. In this email, a conversation log and an assumed case of the corresponding part in the specific case, a probability of occurrence of the case, and a proposal for a solution to the case, and the like may be described. In addition, the robot 100 stores a result of the executed action in the storage unit 220. Specifically, the memory control unit 238 stores, in the history data 222, whether or not the specific case has occurred, the resolution status, and the like. In this way, by feeding back whether or not the specific case has occurred, the solution status, and the like, it is possible to brush up the accuracy of detection of the specific case and the proposal of the solution. Furthermore, regarding “(11) Advice on risks such as “bullying”, “crime”, and “harassment” is given to the user.”, the memory control unit 238 periodically detects the content of conversations that a plurality of users have on the phone or in the company as the states of the users, and stores the content in the history data 222.

Furthermore, the action control unit 250 operates the avatar according to the determined action of the avatar, and displays the avatar in the image display area of the headset-type terminal 820 as the control target 252C. Furthermore, in a case in which the determined action of the avatar includes the utterance content of the avatar, the utterance content of the avatar is output from the speaker as the control target 252C by voice.

In particular, as in the first embodiment, in a case in which the action determination unit 236 determines to give advice on a risk approaching the user 10 such as “bullying”, “crime”, or “harassment” as an action of the avatar, it is preferable to cause the action control unit 250 to control the avatar to give advice on the risk approaching the user 10.

Here, the avatar is, for example, a 3D avatar, and may be selected by the user from avatars prepared in advance, may be a virtual avatar of the user, or may be a favorite avatar generated by the user. To generate an avatar, image generative AI may be utilized to generate an avatar in multiple art styles such as photorealistic, cartoon, moe-style, and oil painting style.

Furthermore, as in the first embodiment, in a case where the action determination unit 236 determines to give advice on a risk approaching the user 10, such as “bullying”, “crime”, or “harassment”, as the action of the avatar, the action determination unit may cause the action control unit 250 to perform control such that the avatar is transformed into another avatar, for example, an avatar that genuinely and empathetically stands by the user 10 such as a family member, a close friend, a teacher, a boss, a colleague, or a counselor of the user 10. In addition, as in the first embodiment, in a case in which the action determination unit 236 determines, as an action of the avatar, to give advice on the risk approaching the user 10 such as “bullying”, “crime” “harassment” or the like, the action determination unit may cause the action control unit 250 to control the avatar to transform into an animal different from a person, for example, a dog, a cat, or the like.

### Sixteenth Embodiment

In the autonomous processing in the embodiment, the robot 100 as an agent has a function as an exclusive trainer for diet or health support for the user 10 in consideration of physical condition management and the like. That is, the robot 100 spontaneously collects information on the daily exercise and meal results of the user 10, and spontaneously acquires all data (voice quality, complexion, heart rate, calorie intake, exercise amount, number of steps, sleeping time, and the like) related to the health of the user 10. Furthermore, while the user 10 lives a daily life, the robot spontaneously presents, to the user 10, compliments, concerns, achievements, and numbers (the number of steps, consumed calories, and the like) regarding health management in a random times. Furthermore, in a case in which a change in physical conditions of the user 10 is sensed from the collected data, a meal or exercise menu corresponding to the situation is proposed, and a light diagnosis is performed.

For example, multiple types of the robot actions include the following (1) to (11).
(1) The robot does nothing.
(2) The robot dreams.
(3) The robot speaks to the user.
(4) The robot creates a picture diary.
(5) The robot proposes an activity.
(6) The robot proposes a person whom the user should meet.
(7) The robot introduces news that the user is interested in.
(8) The robot edits pictures and videos.
(9) The robot studies with the user.
(10) The robot evokes a memory.
(11) The robot gives advice on health to the user.

In a case in which it is determined, as the robot action, to “(11) The robot gives advice on health to the user.”, that is, to give advice on health to the user, the action determination unit 236 determines, based on the event data stored in the history data 222, a content for giving advice to the user 10 regarding the health of the user 10 using the sentence generation model. For example, the action determination unit 236 determines to present, to the user 10, compliment, concern, achievement, and number (the number of steps and calories consumed) regarding health management in a random times while the user 10 lives a daily life. Furthermore, the action determination unit 236 determines to propose a meal or an exercise menu according to a change in physical conditions of the user 10. Furthermore, the action determination unit 236 determines to perform light diagnosis according to a change in physical conditions of the user 10.

Furthermore, regarding the “(11) The robot gives advice on health to the user.”, the related information collection unit 270 collects information regarding meals and exercise menus preferred by the user 10 from external data (web sites such as news sites and moving image sites). Specifically, the related information collection unit 270 acquires and stores the meal and the exercise menu in which the user 10 is interested from an utterance content of the user 10 or a setting operation by the user 10.

Furthermore, regarding “(11) The robot gives advice on health to the user.”, the memory control unit 238 periodically detects data related to exercise, diet, and health of the user as the states of the user, and stores the data in the history data 222. Specifically, the daily exercise and meal results of the user 10 are collected, and all data related to the health of the user 10 such as voice quality, complexion, heart rate, calorie intake, exercise amount, number of steps, and sleeping time are acquired.

In particular, in a case in which the action determination unit 236 determines to give advice on health to the user as the action of the avatar, it is preferable to cause the action control unit 250 to control the avatar so as to determine a content to be advised to the user 10 regarding the health of the user 10 using the sentence generation model based on the event data stored in the history data 222.

For example, the action control unit 250 supports diet of the user 10 by managing meals and exercise while taking into account the physical conditions of the user 10 through an avatar as an exclusive trainer displayed on the headset-type terminal 820 or the like. Specifically, in a random time, for example, a time before a meal or a time before sleep of the user during the daily life of the user 10, the action control unit 250 talks to the user 10 with an expression of compliments or concerns about health management through an avatar or presents a result of dieting to the user 10 as a numerical value (the number of steps or calories consumed). Furthermore, the action control unit 250 proposes a meal or an exercise menu corresponding to a change in the physical conditions of the user 10 to the user 10 through the avatar. Furthermore, the action determination unit 236 performs light diagnosis according to a change in the physical conditions of the user 10 through the avatar. Furthermore, the action control unit 250 assists management of sleep of the user 10 through the avatar.

Here, the avatar is, for example, a 3D avatar, and may be selected by the user from avatars prepared in advance, may be a virtual avatar of the user, or may be a favorite avatar generated by the user.

For example, the avatar may be a virtual avatar of the user having an ideal physique, which is generated based on numerical values such as the target weight, body fat percentage, and BMI of the user 10. That is, in a case in which the action determination unit 236 determines to support diet as the action of the avatar, the action determination unit may operate the avatar to transform into an appearance of the virtual user having the ideal physique. As a result, the user can visually grasp the goal, and the motivation for dieting is maintained. Furthermore, for example, in a case in which the user 10 eats too much or neglects exercise, the action determination unit 236 may cause the action control unit 250 to operate the avatar to transform into a fat appearance of the virtual user. As a result, the user can visually obtain a sense of crisis.

Furthermore, for example, the action control unit 250 may propose to the user 10 to exercise together with the avatar through an avatar that has changed in appearance, such as a model or an athlete admired by the user 10, an instructor of a sports gym, or a popular video distributor distributing videos on exercise. For example, the action control unit 250 may propose to the user 10 to dance together with the avatar by using an avatar that has a transformed appearance, such as a favorite idol or dancer of the user 10, an instructor of a sports gym, or a popular video distributor distributing videos on exercise. Furthermore, for example, the action control unit 250 may propose to the user 10 to perform mitt work movements through an avatar with a mitt.

Furthermore, for example, in a case in which the action determination unit 236 determines to support sleep management, the action control unit 250 may be caused to operate the avatar to transform into the appearance of a plurality of sheep. This induces sleepiness of the user 10.

To generate an avatar, image generative AI may be utilized to generate an avatar in multiple art styles such as photorealistic, cartoon, moe-style, and oil painting style.

### Seventeenth Embodiment

In the autonomous processing in the embodiment, the agent spontaneously collects all information related to the user. For example, in a case in which the user is at home, the agent grasps when and what kind of question the user would ask the agent, and when and what kind of action the user would take (wake up at 7:00 in the morning, turn on the television, check the weather with the smartphone, check the train time with the train line information around 8:00, and the like). Since the agent spontaneously collects various information related to the user, even if the content of the question is unclear just due to the user uttering “train” around 8:00 in the morning, the agent automatically converts the question into a correct question according to need analysis that can be inferred from words or expressions.

For example, multiple types of the robot actions include the following (1) to (11).
(1) The robot does nothing.
(2) The robot dreams.
(3) The robot speaks to the user.
(4) The robot creates a picture diary.
(5) The robot proposes an activity.
(6) The robot proposes a person whom the user should meet.
(7) The robot introduces news that the user is interested in.
(8) The robot edits pictures and videos.
(9) The robot studies with the user.
(10) The robot evokes a memory.
(11) Speech of the user is converted into a question and answered.

The action determination unit 236 automatically converts speech of the user into a correct question and presents the solution of “(11) Speech of the user is converted into a question and answered.”, that is, as a robot action, even if the content is unclear.

Furthermore, regarding the “(11) Speech of the user is converted into a question and answered.”, the memory control unit 238 periodically detects an action of the user as a state of the user and stores the detected action in the history data 222 with time. In addition, the memory control unit 238 may store information on the periphery of the installation place of the agent in the history data 222.

In the embodiment, the control unit 228B has the functions of determining an action of the avatar and generating display of the avatar to be presented to the user through the headset-type terminal 820.

The action recognition unit 234 of the control unit 228B periodically recognizes an action of the user 10 based on the information analyzed by the sensor module unit 210 and the state of the user 10 recognized by the state recognition unit 230, and stores the state of the user 10 including the action of the user 10 in the history data 222.

The action recognition unit 234 of the control unit 228B spontaneously collects all information related to the user 10. For example, in a case in which the user is at home, the action recognition unit 234 grasps when and what kind of question the user 10 would ask to an avatar, and when and what kind of action (wake up at 7:00 in the morning, turn on the television, check the weather with the smartphone, and check the train time with the train line information around 8:00) the user would take.

As in the first embodiment, the emotion determination unit 232 of the control unit 228B determines an emotion value of the agent based on the state of the headset-type terminal 820, and substitutes the emotion value as an emotion value of the avatar.

As in the first embodiment, when an agent functioning as an avatar performs an autonomous process of autonomously acting, the action determination unit 236 of the control unit 228B determines, as an action of the avatar, any of multiple types of avatar actions including not acting, using at least one of the state of the user 10, the emotion of the user 10, the emotion of the avatar, or the state of electronic equipment (for example, the headset-type terminal 820) that controls the avatar, and the action determination model 221, at a predetermined timing.

Specifically, the action determination unit 236 inputs data indicating at least one of a state of the user 10, a state of electronic equipment, an emotion of the user 10, or an emotion of an avatar, together with data for asking about an avatar action to the data generation model, and determines an action of the avatar based on an output of the data generation model.

In addition, the action control unit 250 displays the avatar in the image display area of the headset-type terminal 820 as the control target 252C according to the determined action of the avatar. Furthermore, in a case in which the determined action of the avatar includes the utterance content of the avatar, the utterance content of the avatar is output from the speaker as the control target 252C by voice.

In particular, since the action recognition unit 234 spontaneously collects various information related to the user 10, the action determination unit 236 automatically performs “(11) Converting speech of the user into a question and providing an answer” as an action of the avatar on the AR (VR), for example, a conversion into a correct question using the sentence generation model based on needs analysis that can be inferred from words or expressions, the event data stored in the history data 222, and the state of the user 10 even if the user 10 merely utters “train” around 8:00 in the morning and the content of the question is unclear.

Furthermore, for example, in a case in which an avatar on AR (VR) is set in a mall such as Aeon Mall (registered trademark), the action determination unit 236 ascertains, as an action of the avatar, when and what kind of question the user 10 would ask the avatar. For example, the action determination unit 236 ascertains, as an action of the avatar, that a large number of users 10 would ask where they can buy umbrellas in the evening in the rainy time. Then, only when another user 10 just says “umbrella”, the action determination unit 236 ascertains, as an action of the avatar, the content of the question and presents a solution, thereby realizing a conversion from a mere response of “answering” into a “conversation” with consideration. Furthermore, in this autonomous processing, information of the periphery of the installation place of the avatar is input, and an answer corresponding to the place is created. Whether the question has been solved is checked with the partner, and the correctness/incorrectness of the question and answer is fed back, thereby permanently increasing the resolution rate.

Furthermore, as a plurality of types of avatar actions, “(12) The avatar is transformed into another avatar having a different appearance.” may be further included. In a case in which the action determination unit 236 determines that “(12) The avatar is transformed into another avatar having a different appearance.” as an action of the avatar, it is preferable to cause the action control unit 250 to control the avatar to transform into another avatar. The other avatar includes, for example, an appearance that matches hobbies of the user 10, for example, a face, clothes, hairstyle, and belongings. If the user 10 has various hobbies, the action control unit 250 may be caused to control the avatar to transform into various different avatars in accordance with the hobbies.

### Eighteenth Embodiment

In the autonomous processing in the embodiment, the robot 100 as an agent spontaneously collects various information from an information source such as a television or the web even when the user is absent. For example, in a case in which the robot 100 is still a child, that is, when the robot 100 is still in the activation beginning stage as an example, the robot 100 can hardly have a conversation. However, since the robot 100 always obtains various information when the user is absent, the robot 100 can learn and grow by itself. Therefore, the robot 100 gradually speaks in human language. As an example, the robot 100 initially generates animal sound (voice), but in a case in which a certain condition is exceeded, the robot 100 will come to learn human language and speak in human language.

In a case in which the user raises the robot 100 with which the user can obtain a game-like sense as if a pet talking to the user comes to the user's house, the robot 100 spontaneously learns and gradually memorizes language even when the user is absent. Then, for example, when the user comes home from school, the robot 100 itself utters a conversation “I memorized 10 words today. Apple, koala, egg, . . . ” to the user, which will be a game in which a more realistic robot 100 is raised.

For example, multiple types of robot actions include the following (1) to (12).
(1) The robot does nothing.
(2) The robot dreams.
(3) The robot speaks to the user.
(4) The robot creates a picture diary.
(5) The robot proposes an activity.
(6) The robot proposes a person whom the user should meet.
(7) The robot introduces news that the user is interested in.
(8) The robot edits pictures and videos.
(9) The robot studies with the user.
(10) The robot evokes a memory.
(11) The robot increases its vocabulary.
(12) The robot utters the increased vocabulary.

In a case in which the action determination unit 236 determines, as a robot action, “(11) The robot increases its vocabulary.”, that is, to increase a vocabulary, the robot 100 increases a vocabulary by itself and gradually learns human language even when the user is absent.

Furthermore, regarding the “(11) The robot increases its vocabulary.”, the related information collection unit 270 spontaneously collects various information including a vocabulary by accessing an information source such as a television, the web, or the like by itself even when the user is absent. Furthermore, regarding the “(11) The robot increases its vocabulary.”, the memory control unit 238 stores various vocabularies based on the information collected by the related information collection unit 270.

Note that, in the embodiment, in a case in which the action determination unit 236 determines “(11) The robot increases its vocabulary.” as a robot action, the robot 100 evolves its language to speak in by increasing its own vocabulary even when the user is absent. That is, the vocabulary of the robot 100 is improved. Specifically, initially, the robot 100 generates animal sound (voice), but gradually evolves and utters human language according to the number of vocabularies collected by the robot 100 itself. As an example, for example, the level from animal communication to the speech of adult humans is associated with the cumulative value of the number of vocabularies, and the robot 100 speaks in language of the age corresponding to the cumulative value by itself.

For example, in a case in which the robot 100 first generates a dog's sound, the dog's sound can be evolved into a human language according to the cumulative value of the stored vocabulary, and finally the human language can be uttered. As a result, the user 10 can feel the process in which the robot 100 self-evolves from a dog to a human, that is, a process of self-growth. Furthermore, when the robot 100 speaks in human language, the user 10 can get a sense as if a talking pet has come to the user's home.

Note that the initial voice emitted by the robot 100 enables the user 10 to set a favorite animal of the user 10, such as a dog, a cat, and a bear. In addition, the animal set in the robot 100 can be changed at a desired level. In a case in which an animal is reset, the language uttered by the robot 100 can be reset at the initial stage, or the level when an animal is reset can be maintained.

In a case in which the action determination unit 236 determines, as a robot action, “(12) The robot utters the increased vocabulary.”, that is, to utter increased vocabulary, the robot 100 utters the vocabulary collected and increased by itself. Specifically, the robot utters the vocabulary collected by itself to the user from when the user is absent until the user returns home or comes back. As an example, for example, the robot 100 itself utters a conversation saying “I memorized 10 words today, such as apple, koala, egg, . . . ” to the user who has come home or returned.

In particular, as in the first embodiment, in a case in which the action determination unit 236 increases the vocabulary and determines to utter about the increased vocabulary as an action of the avatar, it is preferable to increase the vocabulary using the output of the action determination model 221 and cause the action control unit 250 to control the avatar to utter about the increased vocabulary.

Here, the avatar is, for example, a 3D avatar, and may be selected by the user from avatars prepared in advance, may be a virtual avatar of the user, or may be a favorite avatar generated by the user. To generate an avatar, image generative AI may be utilized to generate an avatar in multiple art styles such as photorealistic, cartoon, moe-style, and oil painting style.

Furthermore, as in the first embodiment, the action determination unit 236 may increase the vocabulary using the output of the action determination model 221 as an action of the avatar, and in a case in which it is determined to utter about the increased vocabulary, the action determination unit may control the action control unit 250 to change at least one of the face, the body, or the voice of the avatar according to the number of increased vocabularies. The avatar may imitate a real person, an imaginary person, or a character. Specifically, the action control unit 250 may be caused to control the avatar so as to increase the vocabulary and transform an avatar that utters the increased vocabulary into, for example, an avatar having at least one of a face, a body, or a voice of the age corresponding to the cumulative value of the number of vocabularies. In addition, as in the first embodiment, in a case in which the action determination unit 236 determines, as an action of the avatar, to increase vocabulary by using an output of the action determination model 221 and utter about the increased vocabulary, the action determination unit may cause the action control unit 250 to control the avatar to transform into an animal different from a person, for example, a dog, a cat, a bear, or the like. At this time, the action determination unit 236 may control the action control unit 250 such that the age of the animal also becomes the age corresponding to the cumulative value of the number of vocabularies.

### Nineteenth Embodiment

The autonomous processing in the embodiment includes an utterance voice quality switching function.

In other words, in the utterance voice quality switching function, the agent itself can access various webs, news, moving images, and movies, which are information sources, and can store utterances of various speakers (utterance method, voice quality, voice tone, etc.).

The stored information (the voice of another person collected from the information sources) can be rendered to increase so-called repertoire in one's own voice one after another by using the voice generation AI. As a result, the voice to be emitted can be changed according to the attributes of the user (child, adult, doctor, teacher, physician, student, minor, company director, and the like).

As a result, for example, if the user is a child, the voice should sound cute. If the user is a physician, the voice should sound like an actor or an announcer. If the user is a company director, the voice should sound like an executive. If the user is from the Kansai region, the agent will automatically switch itself to the Kansai dialect.

For example, multiple types of robot actions include the following (1) to (12).
(1) The robot does nothing.
(2) The robot dreams.
(3) The robot speaks to the user.
(4) The robot creates a picture diary.
(5) The robot proposes an activity.
(6) The robot proposes a person whom the user should meet.
(7) The robot introduces news that the user is interested in.
(8) The robot edits pictures and videos.
(9) The robot studies with the user.
(10) The robot evokes a memory.
(11) A robot utterance method is learned.
(12) The settings of the robot utterance method are changed.

In a case in which the action determination unit 236 determines, as a robot action, “(11) A robot utterance method is learned”, that is, to learn an utterance method (for example, a voice emitted), the action determination unit uses the voice generative AI to sequentially increase so-called repertoire as one's own voice.

Furthermore, regarding “(11) A robot utterance method is learned”, the related information collection unit 270 collects information by accessing various web news, moving images, and movies by itself.

Furthermore, regarding “(11) A robot utterance method is learned”, the memory control unit 238 stores utterance methods, voice qualities, tone, and the like of various speakers based on the information collected by the related information collection unit 270.

Meanwhile, in a case in which the action determination unit 236 determines that “(12) The settings of the robot utterance method are changed”, that is, the robot 100 makes an utterance, as a robot action, the robot 100 itself switches the voice to the cute voice if the user is a child, switches the voice to the voice of an actor or an announcer if the user is a doctor, switches the voice to the voice of a CEO if the user is a company director, and switches the voice to the Kansai dialect if the user is a Kansai person. Note that the utterance method includes a language, and in a case in which it is recognized that the interaction partner is studying a foreign language such as English, French, German, Spanish, Korean, or Chinese, the interaction may be performed in the foreign language being studied.

For example, it would also be possible to adopt a configuration where a white dog plush toy, like a Hokkaido dog, is used as a specific character, anthropomorphized (e.g., as a father) to be positioned as a family member, and a drive system and control system (walking system) for moving around indoors are synchronized with a control system (agent system) governing conversation and actions, thereby linking movement and conversation. In this case, the voice of the father is default for the white dog, but the utterance method (dialect, language, etc.) may be changed depending on the interaction partner based on the utterance of another person collected from the information source as the action of the white dog ((11) and (12) of the robot actions described above).

In particular, in a case in which the action determination unit 236 determines to make an utterance as an action of the avatar, it is preferable to cause the action control unit 250 to control the avatar to utter in a changed voice in accordance with the attributes of the user (child, adult, doctor, teacher, physician, student, minor, company director, or the like).

Here, a feature of the embodiment is that the action that can be executed by the robot 100 described in the above-described embodiment is reflected in the action of the avatar displayed in the image display area of the headset-type terminal 820. Hereinafter, when simply referred to as an “avatar”, it is assumed to indicate an avatar that is controlled by the action control unit 250 and displayed in the image display area of the headset-type terminal 820.

That is, the control unit 228B illustrated in FIG. 15 has an utterance voice quality switching function for the avatar when determining an action of the avatar and displaying the avatar to be presented to the user through the headset-type terminal 820.

In other words, in the utterance voice quality switching function, it is possible to access various webs, news, moving images, and movies, which are information sources, and to store utterances of various speakers (utterance method, voice quality, voice tone, etc.).

The stored information (the voice of another person collected from the information sources) can be rendered to increase so-called repertoire in the avatar's own voice one after another by using the voice generation AI. As a result, the voice at the time of utterance can be changed according to attributes of the user.

This allows the agent itself to automatically switch, for example, to a cute voice if the user is a child, a voice like an actor or announcer if the user is a doctor, the voice of a CEO if the user is a company director, or Kansai dialect if the user is from the Kansai region.

In a case in which the action determination unit 236 determines to learn an utterance method (for example, voice emitted) (which corresponds to replacing “(11) A robot utterance method is learned.” of the first embodiment with “(11) An avatar utterance method is learned.”) as an action of the avatar, the so-called repertoire is increased one after another as its own voice using the voice generation AI.

Furthermore, in the embodiment, when the utterance method of the avatar controlled by the action control unit 250 is learned, the related information collection unit 270 itself accesses various web news, moving images, and movies and collects information.

Furthermore, the memory control unit 238 stores utterance methods, voice qualities, tones, and the like of various speakers based on the information collected by the related information collection unit 270.

On the other hand, in a case in which the action determination unit 236 determines to change the avatar's utterance method settings (corresponding to changing the setting from “(12) Settings for the robot utterance method are changed” to “(12) Settings for the avatar utterance method are changed” in the first embodiment), it will then, for example, switch to a cute voice if the user is a child, a voice like an actor or announcer if the user is a doctor, the voice of a CEO if the user is a company director, or Kansai dialect if the user is from the Kansai region. This switching of utterance methods is controlled by the action control unit 250 and is performed by the avatar itself.

Note that the utterance method includes a language, and in a case in which it is recognized that the interaction partner is studying a foreign language such as English, French, German, Spanish, Korean, or Chinese, the interaction may be performed in the foreign language being studied.

Furthermore, in a case in which it is determined to change the settings for the utterance method as an action of the avatar, the action control unit 250 may operate the avatar with the appearance corresponding to the voice emitted after the change.

Here, the avatar is, for example, a 3D avatar, and may be selected by the user from avatars prepared in advance, may be a virtual avatar of the user, or may be a favorite avatar generated by the user.

Furthermore, the avatar displayed in the image display area of the headset-type terminal 820 can be deformed, and for example, it would also be possible to adopt a configuration where the avatar transforms into a specific character, such as a white dog like a Hokkaido dog, is anthropomorphized (e.g., as a father) to be positioned as a family member, and a drive system and control system (walking system) for moving around indoors are synchronized with a control system (agent system) governing conversation and actions, thereby linking movement and conversation.

In this case, the white dog is basically the voice of the father, but as the behavior of the white dog ((11) and (12) of the avatar action described above), the utterance method (dialect, language, and the like) may be changed depending on the interaction partner based on the utterance of another person collected from the information sources.

Note that the transformation of the avatar is not limited to an organism such as an animal or a plant, and the avatar may be transformed into an electrical appliance, or may be transformed into a device such as a tool, an instrument, or a machine, and a still object such as a vase, a bookshelf, or an artwork.

Furthermore, the avatar displayed in the image display area of the headset-type terminal 820 may execute an operation disregarding physical laws (teleportation, double speed movement, and the like).

### Twentieth Embodiment

The autonomous processing in the embodiment includes an utterance voice quality switching function.

In other words, in the utterance voice quality switching function, the agent itself can access various webs, news, moving images, and movies, which are information sources, and can store utterances of various speakers (utterance method, voice quality, voice tone, etc.).

The stored information (the voice of another person collected from the information sources) can be rendered to increase so-called repertoire in one's own voice one after another by using the voice generation AI. As a result, the voice to be emitted can be changed according to the attributes of the user (child, adult, doctor, teacher, physician, student, minor, company director, and the like).

As a result, for example, if the user is a child, the voice should sound cute. If the user is a physician, the voice should sound like an actor or an announcer. If the user is a company director, the voice should sound like an executive. If the user is from the Kansai region, the agent will automatically switch itself to the Kansai dialect.

For example, multiple types of robot actions include the following (1) to (12).
(1) The robot does nothing.
(2) The robot dreams.
(3) The robot speaks to the user.
(4) The robot creates a picture diary.
(5) The robot proposes an activity.
(6) The robot proposes a person whom the user should meet.
(7) The robot introduces news that the user is interested in.
(8) The robot edits pictures and videos.
(9) The robot studies with the user.
(10) The robot evokes a memory.
(11) A robot utterance method is learned.
(12) The settings of the robot utterance method are changed.

In a case in which the action determination unit 236 determines, as a robot action, “(11) A robot utterance method is learned”, that is, to learn an utterance method (for example, a voice emitted), the action determination unit uses the voice generative AI to sequentially increase so-called repertoire as one's own voice.

Furthermore, regarding “(11) A robot utterance method is learned”, the related information collection unit 270 collects information by accessing various web news, moving images, and movies by itself.

Furthermore, regarding “(11) A robot utterance method is learned”, the memory control unit 238 stores utterance methods, voice qualities, tone, and the like of various speakers based on the information collected by the related information collection unit 270.

Meanwhile, in a case in which “(12) The settings of the robot utterance method are changed”, that is, that the robot 100 utters is determined as a robot action, the robot 100 itself switches the voice to the cute voice if the user is a child, switches the voice to the voice of an actor or an announcer if the user is a doctor, switches the voice to the voice of a CEO if the user is a company director, and switches the voice to the Kansai dialect if the user is a Kansai person. Note that the utterance method includes a language, and in a case in which it is recognized that the interaction partner is studying a foreign language such as English, French, German, Spanish, Korean, or Chinese, the interaction may be performed in the foreign language being studied.

For example, it would also be possible to adopt a configuration where a white dog plush toy, like a Hokkaido dog, is used as a specific character, anthropomorphized (e.g., as a father) to be positioned as a family member, and a drive system and control system (walking system) for moving around indoors are synchronized with a control system (agent system) governing conversation and actions, thereby linking movement and conversation. In this case, the voice of the father is default for the white dog, but the utterance method (dialect, language, etc.) may be changed depending on the interaction partner based on the utterance of another person collected from the information source as the action of the white dog ((11) and (12) of the robot actions described above).

In particular, in a case in which the action determination unit 236 determines to make an utterance as an action of the avatar, it is preferable to cause the action control unit 250 to control the avatar to utter in a changed voice in accordance with the attributes of the user (child, adult, doctor, teacher, physician, student, minor, company director, or the like).

Here, the feature of the embodiment is that the action that can be executed by the robot 100 described in the first embodiment is reflected in the action of the avatar displayed in the image display area of the headset-type terminal 820. Hereinafter, when simply referred to as an “avatar”, it is assumed to indicate an avatar that is controlled by the action control unit 250 and displayed in the image display area of the headset-type terminal 820.

That is, the control unit 228B illustrated in FIG. 15 has an utterance voice quality switching function for the avatar when determining an action of the avatar and displaying the avatar to be presented to the user through the headset-type terminal 820.

In other words, in the utterance voice quality switching function, it is possible to access various webs, news, moving images, and movies, which are information sources, and to store utterances of various speakers (utterance method, voice quality, voice tone, etc.).

The stored information (the voice of another person collected from the information sources) can be rendered to increase so-called repertoire in the avatar's own voice one after another by using the voice generation AI. As a result, the voice at the time of utterance can be changed according to attributes of the user.

This allows the agent itself to automatically switch, for example, to a cute voice if the user is a child, a voice like an actor or announcer if the user is a doctor, the voice of a CEO if the user is a company director, or Kansai dialect if the user is from the Kansai region.

In a case in which the action determination unit 236 determines to learn an utterance method (for example, voice emitted) (which corresponds to replacing “(11) A robot utterance method is learned.” of the first embodiment with “(11) An avatar utterance method is learned.”) as an action of the avatar, the so-called repertoire is increased one after another as its own voice using the voice generation AI.

Furthermore, in the embodiment, when the utterance method of the avatar controlled by the action control unit 250 is learned, the related information collection unit 270 itself accesses various web news, moving images, and movies and collects information.

Furthermore, the memory control unit 238 stores utterance methods, voice qualities, tones, and the like of various speakers based on the information collected by the related information collection unit 270.

On the other hand, in a case in which the action determination unit 236 determines to change the avatar's utterance method settings (corresponding to changing the setting from “(12) Settings for the robot utterance method are changed” to “(12) Settings for the avatar utterance method are changed” in the first embodiment), it will then, for example, switch to a cute voice if the user is a child, a voice like an actor or announcer if the user is a doctor, the voice of a CEO if the user is a company director, or Kansai dialect if the user is from the Kansai region.

This switching of utterance methods is controlled by the action control unit 250 and is performed by the avatar itself.

Note that the utterance method includes a language, and in a case in which it is recognized that the interaction partner is studying a foreign language such as English, French, German, Spanish, Korean, or Chinese, the interaction may be performed in the foreign language being studied.

Furthermore, in a case in which it is determined to change the settings for the utterance method as an action of the avatar, the action control unit 250 may operate the avatar with the appearance corresponding to the voice emitted after the change.

Here, the avatar is, for example, a 3D avatar, and may be selected by the user from avatars prepared in advance, may be a virtual avatar of the user, or may be a favorite avatar generated by the user.

Furthermore, the avatar displayed in the image display area of the headset-type terminal 820 can be deformed, and for example, it would also be possible to adopt a configuration where the avatar transforms into a specific character, such as a white dog like a Hokkaido dog, is anthropomorphized (e.g., as a father) to be positioned as a family member, and a drive system and control system (walking system) for moving around indoors are synchronized with a control system (agent system) governing conversation and actions, thereby linking movement and conversation.

In this case, the white dog is basically the voice of the father, but as the behavior of the white dog ((11) and (12) of the avatar action described above), the utterance method (dialect, language, and the like) may be changed depending on the interaction partner based on the utterance of another person collected from the information sources.

Note that the transformation of the avatar is not limited to an organism such as an animal or a plant, and the avatar may be transformed into an electrical appliance, or may be transformed into a device such as a tool, an instrument, or a machine, and a still object such as a vase, a bookshelf, or an artwork.

Furthermore, the avatar displayed in the image display area of the headset-type terminal 820 may execute an operation disregarding physical laws (teleportation, double speed movement, and the like).

### Twenty-First Embodiment

In the autonomous processing in the embodiment, as an example, the robot 100 as an agent grasps all the conversations and movements of the child who is the user 10, and always calculates (estimates) the mental age of the user 10 from the conversations and movements of the user. Then, the robot 100 spontaneously has a conversation with the user 10 in accordance with the mental age of the user 10, thereby realizing communication as a family in consideration of words according to the growth of the user 10 and the content of the past conversation with the user 10. Furthermore, the words uttered by the robot 100 and the operation and function of the robot 100 are expanded in accordance with the increase in the mental age of the user 10, and the robot 100 spontaneously considers an item that the robot can do together with the user 10 and spontaneously proposes (utters) to the user 10, thereby supporting the capability development of the user 10 as an older brother or sister.

For example, multiple types of robot actions include the following (1) to (12).
(1) The robot does nothing.
(2) The robot dreams.
(3) The robot speaks to the user.
(4) The robot creates a picture diary.
(5) The robot proposes an activity.
(6) The robot proposes a person whom the user should meet.
(7) The robot introduces news that the user is interested in.
(8) The robot edits pictures and videos.
(9) The robot studies with the user.
(10) The robot evokes a memory.
(11) The robot estimates the mental age of the user.
(12) The robot considers the mental age of the user.

In a case in which it is determined, as a robot action, that “(11) The robot estimates the mental age of the user.”, that is, to estimate the mental age of the user 10 based on the action of the user 10, the action determination unit 236 estimates the mental age of the user 10 based on the action (conversation or movement) of the user 10 recognized by the state recognition unit 230. At this time, the action determination unit 236 may estimate the mental age of the user 10 by inputting the action of the user 10 recognized by the state recognition unit 230 to a neural network trained in advance and evaluating the mental age of the user 10, for example.

Furthermore, the action determination unit 236 may periodically detect (recognize) an action (conversation or movement) of the user 10 by the state recognition unit 230 as a state of the user 10, store the action in the history data 222, and estimate the mental age of the user 10 based on the action of the user 10 stored in the history data 222. Furthermore, for example, the action determination unit 236 may estimate the mental age of the user 10 by comparing the recent action of the user 10 stored in the history data 222 with the past action of the user 10 stored in the history data 222.

In a case in which “(12) The robot considers the mental age of the user 10.” is determined, that is, the estimated mental age of the user 10 is considered to determine the action of the robot 100, as a robot action, for example, the action determination unit 236 determines a word emitted by the robot 100, a way of speaking, and movement (changes movement) with respect to the user 10 according to (tailored to) the estimated mental age of the user 10. Specifically, for example, as the estimated mental age of the user 10 increases, the action determination unit 236 increases the difficulty level of words uttered by the robot 100, or brings the way of speaking and the movement of the robot 100 closer to those of adults. Furthermore, the action determination unit 236 may increase the types of words and movement of the robot 100 to the user 10 or extend the function of the robot 100 as the mental age of the user 10 increases. Furthermore, the action determination unit 236 may input a text indicating the mental age of the user 10 to the sentence generation model in addition to a text indicating at least one of the state of the user 10, the emotion of the user 10, the emotion of the robot 100, or the state of the robot 100 and a text for asking about the action of the robot 100, and determine the action of the robot 100 based on the output of the sentence generation model, for example.

Furthermore, the action determination unit 236 may cause the robot 100 to spontaneously utter to the user 10 in accordance with, for example, the mental age of the user 10. Furthermore, the action determination unit 236 may estimate an item that the robot 100 can do together with the user 10 according to the mental age of the user 10, and spontaneously propose (utter) the estimation to the user 10. Furthermore, the action determination unit 236 may extract (select) a conversation content or the like according to the mental age of the user 10 from the conversation content or the like between the user 10 and the robot 100 stored in the history data 222, and add the conversation content or the like to the utterance content of the robot 100 for the user 10, for example.

Particularly, in a case in which it is determined, as an avatar action, that “(11) The avatar estimates the mental age of the user.”, that is, to estimate the mental age of the user 10 based on the action of the user 10, the action determination unit 236 estimates the mental age of the user 10 based on the action (conversation or movement) of the user 10 recognized by the state recognition unit 230. At this time, the action determination unit 236 may estimate the mental age of the user 10 by inputting the action of the user 10 recognized by the state recognition unit 230 to a neural network trained in advance and evaluating the mental age of the user 10, for example. Furthermore, the action determination unit 236 may periodically detect (recognize) an action (conversation or movement) of the user 10 by the state recognition unit 230 as a state of the user 10, store the action in the history data 222, and estimate the mental age of the user 10 based on the action of the user 10 stored in the history data 222. Furthermore, for example, the action determination unit 236 may estimate the mental age of the user 10 by comparing the recent action of the user 10 stored in the history data 222 with the past action of the user 10 stored in the history data 222.

Furthermore, in a case in which the action determination unit 236 determines, as an avatar action, “(12) The avatar considers the mental age of the user 10.”, that is, determines to determine an action of the avatar in consideration of the mental age of the user 10, for example, it is preferable to cause the action control unit 250 to control the avatar such that words uttered by the avatar to the user 10 or the way of speaking and movements of the avatar with respect to the user 10 are changed in accordance with (tailored to) the estimated mental age of the user 10.

Specifically, for example, as the estimated mental age of the user 10 increases, the action determination unit 236 increases the difficulty level of words uttered by the avatar, or brings the way of speaking and the movements of the avatar closer to those of adults. Furthermore, the action determination unit 236 may increase the types of words and movements emitted by the avatar to the user 10 or extend the function of the avatar as the mental age of the user 10 increases. Furthermore, the action determination unit 236 may input a text indicating the mental age of the user 10 to the sentence generation model in addition to a text indicating at least one of the state of the user 10, the emotion of the user 10, the emotion of the avatar, or the state of the avatar and a text for asking about the action of the avatar, and determine the action of the avatar based on the output of the sentence generation model, for example.

Furthermore, the action determination unit 236 may cause the avatar to spontaneously utter to the user 10 in accordance with, for example, the mental age of the user 10. Furthermore, the action determination unit 236 may estimate an item that the avatar can do together with the user 10 according to the mental age of the user 10, and spontaneously propose (utter) the estimation to the user 10. Furthermore, the action determination unit 236 may extract (select) a conversation content or the like according to the mental age of the user 10 from the conversation content or the like between the user 10 and the avatar stored in the history data 222, and add the conversation content or the like to the utterance content of the avatar for the user 10, for example.

Furthermore, the action control unit 250 may change the appearance of the avatar in accordance with the mental age of the user 10. In other words, the action control unit 250 may cause the appearance of the avatar to grow or switch the avatar to another avatar having a different appearance as the mental age of the user 10 increases.

### Twenty-Second Embodiment

In the autonomous processing in the embodiment, the robot 100 as an agent constantly remembers and detects the English ability of the user 10 as a student, and grasps the English level of the user 10. The vocabulary available for use is determined by one's English level. For this reason, the robot 100 does not use a word at a higher level than the English level of the user 10, or the like, and can spontaneously speak in English to match the English level of the user 10 at all times. Furthermore, in order to lead the user 10 to improvement in English in the future, a lesson program tailored to the user 10 is also devised, and English conversations are advanced by subtly mixing in words that are one level higher so as to improve the user's English. Note that the foreign language is not limited to English, and may be another language.

For example, multiple types of the robot actions include the following (1) to (11).
(1) The robot does nothing.
(2) The robot dreams.
(3) The robot speaks to the user.
(4) The robot creates a picture diary.
(5) The robot proposes an activity.
(6) The robot proposes a person whom the user should meet.
(7) The robot introduces news that the user is interested in.
(8) The robot edits pictures and videos.
(9) The robot studies with the user.
(10) The robot evokes a memory.
(11) The robot estimates the English level of the user.
(12) The robot conducts an English conversation with the user.

In a case in which it is determined, as a robot action, that “(11) The robot estimates the English level of the user.”, that is, the English level of the user 10 is estimated, the action determination unit 236 estimates the English level of the user 10 from the level of the English words used by the user 10, the appropriateness of the English words with respect to the contexts, the length of the sentences or the accuracy of the grammar spoken by the user 10, the speaking speed and fluency of the user 10, the comprehension level (listening ability) of the user 10 with respect to the content spoken in English by the robot 100, and the like, based on the conversation with the user 10 stored in the history data 222.

In a case in which it is determined that “(12) The robot conducts English conversations with the user”, that is, determined to have an English conversation with the user, as a robot action, the action determination unit 236 determines a content to be uttered to the user 10 by using the sentence generation model based on the event data stored in the history data 222. At this time, the action determination unit 236 performs the English conversation according to the level of the user 10. Furthermore, a lesson program tailored to the user 10 is created so as to lead the user 10 to improvement in English in the future, and a conversation with the user 10 is conducted based on the program. Furthermore, the action determination unit 236 advances the conversation by subtly mixing in English words that are one level higher so that the English ability of the user 10 improves.

In addition, regarding “(12) The robot conducts an English conversation with the user.”, the related information collection unit 270 collects preferences of the user 10 from external data (web sites such as news sites and moving image sites). Specifically, the related information collection unit 270 acquires news and hobby topics that the user 10 shows interest in from utterance contents of the user 10 or a setting operation by the user 10 in advance. In addition, the related information collection unit 270 also collects, from the external data, the English words one level higher than the English level of the user 10.

Furthermore, regarding “(12) The robot conducts an English conversation with the user.”, the memory control unit 238 always stores and detects the English ability of the user 10 as a student.

Particularly, in a case in which the action determination unit 236 determines to estimate the English level of the user as an action of the avatar, it is preferable for the action determination unit 236 to cause the action control unit 250 to control the avatar to estimate the English level of the user 10 from the level of the English words used by the user 10, the appropriateness of the English words with respect to the contexts, the length of the sentences or the accuracy of the grammar spoken by the user 10, the speaking speed and fluency of the user 10, the comprehension level (listening ability) of the user 10 with respect to the content spoken in English by the avatar, and the like, based on the conversation with the user 10 stored in the history data 222. As a result, the avatar always grasps the English level of the user 10 as a student.

In addition, in a case in which the action determination unit 236 determines to conduct an English conversation with the user as an action of the avatar, it is preferable for the action determination unit 236 to cause the action control unit 250 to control the avatar to determine a content to be uttered to the user 10 by the avatar and conduct an English conversation with the user 10 tailored to the level of the user by using the sentence generation model based on the event data stored in the history data 222.

For example, the action control unit 250 does not use words at a higher level than the English level of the user 10 or the like through an avatar displayed on the headset-type terminal 820 or the like, and always conducts English conversations tailored to the English level of the user 10. Furthermore, for example, the action control unit 250 creates a lesson program tailored to the user 10 so as to lead the user 10 to improvement in English conversation in the future, and conducts English conversations with the user 10 through the avatar based on the program. Furthermore, the action control unit 250 advances the English conversation through the avatar by subtly mixing in English words that are one level higher than the current level of the user so that the English ability of the user 10 improves. Note that the foreign language is not limited to English, and may be another language.

Here, the avatar is, for example, a 3D avatar, and may be selected by the user from avatars prepared in advance, may be a virtual avatar of the user, or may be a favorite avatar generated by the user.

For example, the action control unit 250 may conduct English conversations with the user 10 through an avatar having a changed appearance of a person from an English-speaking country. Furthermore, for example, in a case in which the user 10 desires to learn business English, the action control unit 250 may conduct English conversations with the user 10 through an avatar wearing a suit. Furthermore, for example, the action control unit 250 may change the appearance of the avatar in accordance with the content of the conversation. For example, the action control unit 250 may create a lesson program for learning famous quotes from great people in history in English, and may have English conversations with the user 10 through an avatar that has changed to the appearance of such a great person.

To generate an avatar, image generative AI may be utilized to generate an avatar in multiple art styles such as photorealistic, cartoon, moe-style, and oil painting style.

### Twenty-Third Embodiment

In the autonomous processing in the embodiment, in a case in which the user 10 is involved in a creative activity, the robot 100 as an agent acquires information necessary for the user 10 from external data (web sites such as news sites and moving image sites, distribution news, and the like). The robot 100 autonomously acquires the information at all times even when the user 10 is absent, that is, even when the user 10 is not around the robot 100. Then, when the user 10, who is involved in creative activities, takes an action and the robot 100 as an agent detects this action, the robot 100 gives a hint for the user 10 to elicit his/her creativity. For example, when the user 10 is visiting a historical building such as an old temple in Kyoto, looking at a scenic spot such as Mt. Fuji, or performing a creation activity such as painting in an atelier, the robot 100 issues a hint useful for eliciting creativity to the user 10. This creativity includes inspiration, i.e., intuitive insights and thoughts. For example, the robot supports creation of works of art by composing a first phrase of a Haiku corresponding to an old temple in Kyoto, presenting the beginning part (or characteristic part) of a novel that can be imagined from a scenery of Mt. Fuji, or making a proposal for enhancing the inventiveness of a painting being drawn. Here, users who are involved in the creative activity include an artist. An artist is a person engaged in creation activities. Artists include persons who produce and create works of art. For example, artists include a sculptor, a painter, a director, a musician, a dancer, a choreographer, a film director, a videographer, a calligrapher (calligraphy artist), a designer, an illustrator, a photographer, an architect, a craft artist, and an author. Furthermore, the artists include a performer, an instrumentalist, and the like. In this case, the robot 100 determines an action that is a hint for enhancing the creativity of an artist. In addition, the robot 100 determines an action that is a hint for enhancing the expressiveness of an artist. The action control unit 250 recognizes an action of the user 10, determines an action of the robot 100 corresponding to the recognized action of the user 10, and controls the control target 252 based on the determined action of the robot 100.

For example, multiple types of the robot actions include the following (1) to (11).
(1) The robot does nothing.
(2) The robot dreams.
(3) The robot speaks to the user.
(4) The robot creates a picture diary.
(5) The robot proposes an activity.
(6) The robot proposes a person whom the user should meet.
(7) The robot introduces news that the user is interested in.
(8) The robot edits pictures and videos.
(9) The robot studies with the user.
(10) The robot evokes a memory.
(11) The robot gives advice on creative activities to the user.

In a case in which the action determination unit 236 determines that, as a robot action, “(11) The robot gives advice on creative activities to the user.”, that is, to give advice on necessary information to the user involved in creative activities, the action determination unit acquires necessary information for the user from external data. The robot 100 autonomously acquires such information at all times even when the user is absent.

Furthermore, regarding “(11) The robot gives advice on creative activities to the user.”, the related information collection unit 270 collects information regarding creative activities of the user as the user's preference information and stores the collected information in the collected data 223.

For example, in a case in which the user goes to an old temple in Kyoto, the related information collection unit 270 obtains, from external data, a haiku corresponding to the old temple and stores the Haiku in the collected data 223. Then, a part of the haiku, for example, the first phrase, is output as audio from a speaker or displayed in text on a display. Furthermore, in a case in which the user sees Mt. Fuji, a passage of a novel that can be imagined from the scenery of Mt. Fuji, for example, the beginning part, is obtained from external data and stored in the collected data 223. Then, the beginning part is output as audio from a speaker or displayed as text on a display. Furthermore, in a case in which the user is drawing a picture in an atelier, information on how to draw a fine picture from then on is obtained from external data based on the picture being drawn, and stored in the collected data 223. Then, this information is output as audio from the speaker or displayed in text on the display, thereby supporting the creation of a work of art of the user.

Note that the information of the user 10 as an artist may include information regarding past performance of the user 10, for example, information regarding a work created by the user 10 in the past, a video or a stage in which the user 10 performed in the past, and the like.

For example, the action determination unit 236 may determine an action that offers a hint for eliciting or enhancing the creativity of the user 10 who is an artist. For example, the action determination unit 236 may determine an action that offers a hint for eliciting inspirational creativity from the user 10. For example, the action determination unit 236 may determine an action that offers a hint for eliciting or enhancing the expressiveness of the user 10 who is an artist. For example, the action determination unit 236 may determine an action that offers a hint for improving self-expression of the user 10.

Particularly, in a case in which the action determination unit 236 determines, as an action of the avatar, to give necessary advice to the user 10 involved in creative activities, the action determination unit collects information about the creative activities of the user 10 and further collects information necessary for advising from external data. Then, it is preferable to determine the content of the advice to be given to the user 10 and cause the action control unit 250 to control the avatar to give the advice.

Here, the avatar is, for example, a 3D avatar, and may be selected by the user from avatars prepared in advance, may be a virtual avatar of the user, or may be a favorite avatar generated by the user. To generate an avatar, image generative AI may be utilized to generate an avatar in multiple art styles such as photorealistic, cartoon, moe-style, and oil painting style.

The action of the avatar that gives advice preferably includes an action of praising the user 10. In other words, aspects worthy of high praise in the user 10's creative activities or in their intermediate work/process are found, and then, the advice explicitly includes these commendable points as specific praise. Since the user 10 is complimented by the advice from the avatar, it is expected that the user will have increased motivation, leading to a new creativity.

The “content of advice” includes advice appealing to the sense of the user 10, for example, vision, hearing, and the like, in addition to advice simply indicated by a sentence (text data). For example, in a case in which the creation activity of the user 10 is an activity related to painting production, advice that visually indicates color usage and composition is included. Furthermore, in a case in which the creative activity of the user 10 is an activity related to music production such as composition or arrangement, advice that aurally indicates a melody, chord progression, or the like using the sound of musical instruments is included.

Furthermore, the “content of advice” includes an expression, a gesture, and the like of the avatar. For example, the advice includes praising with an action including an expression or a gesture in a case in which the action of praising is performed on the user 10. In this case, the action includes replacing the face or a part of the body of the avatar from the original ones of the avatar with other ones. More specifically, the action control unit 250 expresses an expression in which the avatar is happy with the user 10 who has grown up in the creative activity by narrowing the avatar's eyes (replacing the eyes with narrower eyes) or turning the entire expression into a smile. Furthermore, the action control unit 250 may cause the user 10 to recognize that the avatar highly evaluates the creative activity of the user 10 by nodding deeply as a gesture of the avatar.

The “content of advice” may be determined based on not only the creative activity of the user 10 at the time of giving advice, the state of the user 10, the state of the avatar, the emotion of the user 10, and the emotion of the avatar, but also the content of advice given in the past. For example, in a case in which the creative activity of the user 10 can be sufficiently supported by advice given in the past, the action control unit 250 causes the avatar to give advice with different contents next time to offer a hint on new creation to the user 10. On the other hand, in a case in which the advice given in the past cannot sufficiently support the creative activity of the user 10, the avatar gives advice of the same purpose in a different method or viewpoint. More specifically, for example, in a case in which the creative activity of the user 10 is photographing and advice has been given in the past focusing only on the work that was simply completed, the avatar gives advice including a specific operation method of photographing equipment (a camera, a smartphone, or the like) as the next advice. In this case, the action control unit 250 displays an icon of the photographing equipment together with the avatar in the image display area of the headset-type terminal 820. Then, by exemplifying the method of operating the imaging equipment by the avatar while facing the icon of the imaging equipment with a specific operation, it becomes easier for the user 10 to understand the advice. Furthermore, the action control unit 250 may display buttons or switches for operation when the avatar has transformed into imaging equipment.

### Twenty-Fourth Embodiment

In the autonomous processing in the embodiment, the agent may spontaneously or periodically detects an action or state of the user by monitoring the user. Specifically, the agent may detect an action executed by the user at home by monitoring the user. The agent may be interpreted as an agent system to be described later. Hereinafter, the agent system may be simply referred to as an agent.

It may be interpreted that the agent or the robot 100 spontaneously proactively acquires a state of the user without a trigger from outside.

The trigger from outside may include a question from the user to the robot 100, an active action from the user to the robot 100, or the like. The term “periodically” may be interpreted as a specific cycle such as in units of one second, one minute, one hour, several hours, several days, week, or day of the week.

The actions performed by the user at home may include housework, nail clipping, watering plants, personal grooming to go out, walking pets, and the like. The housework may include cleaning the toilet, preparing a meal, cleaning the bath, taking in laundry, cleaning the floor, childcare, shopping, emptying the trash can, ventilating a room, and the like.

In the autonomous processing, the agent may store the detected type of action executed by the user at home as specific information associated with the timing at which the action is executed. Specifically, user information of a user (person) included in a specific home, information indicating the type of action such as housework performed by the user at home, and a past timing at which each of the actions is executed are stored in association with each other. The past timing may be the number of times of execution of at least one or more actions.

In the autonomous processing, the agent may estimate an execution timing, which is a timing at which the user should execute an action, spontaneously or periodically based on the stored specific information, and may give the user a proposal for encouraging the user to take an action based on the estimated execution timing.

Hereinafter, an example of a proposal content to the user by the agent will be described.

(1) In a case in which the husband at home performs nail clipping, the agent monitors the action of the husband to record the past nail clipping operation and record the timings of the execution of the nail clipping (a time point at which the nail clipping is started, a time point at which the nail clipping is ended, and the like). The agent records the past nail clipping operation a plurality of times, thereby estimating the interval (for example, the number of days such as 10 days and 20 days) of the nail clipping by the husband based on the timing at which the nail clipping was performed for each person who has performed nail clipping. In this way, the agent may estimate the execution timing of the next nail clipping by recording the execution timing of the nail clipping, and propose nail clipping to the user when the estimated number of days has elapsed from the time point when the previous nail clipping was executed. Specifically, when 10 days have elapsed since the previous nail clipping, the agent causes electronic equipment to reproduce a voice saying “Would you like to clip nails now?” or “Your nails might be getting long”, thereby proposing that the user should clip nails, which is an action the user can take. The agent may display these messages on the screen of the electronic equipment, instead of reproducing the voices.

(2) In a case in which the wife of the family has watered a plant, the agent monitors the action of the wife to record the past watering operation and record the timing (time point at which watering is started, time point at which watering is finished, and the like) at which the watering has been performed. By recording the past watering operation a plurality of times, the agent estimates a watering interval (for example, the number of days such as 10 days and 20 days) of the wife based on the timing at which watering was performed for each person who has watered. In this way, the agent may estimate the next watering execution timing by recording the watering execution timing, and propose the execution timing to the user when the estimated number of days has elapsed from the time point at which the previous watering is executed. Specifically, the agent proposes watering, which is an action the user can take, to the user by causing the electronic equipment to reproduce a voice saying “Would you like to water now?” or “The water of the plants may be reduced”. The agent may display these messages on the screen of the electronic equipment, instead of reproducing the voices.

(3) In a case in which a child at home cleans a toilet, the agent monitors an action of the child to record a past operation of cleaning the toilet and record the timing at which cleaning of the toilet is performed (the time point at which cleaning of the toilet is started, a time point when cleaning of the toilet is finished, and the like). By recording the past toilet cleaning operation a plurality of times, the agent estimates an interval of the toilet cleaning by the child (for example, the number of days such as 7 days and 14 days) based on the timing at which toilet cleaning was performed for each person who cleans the toilet. In this way, the agent may estimate the execution timing of the next toilet cleaning by recording the execution timing of the toilet cleaning, and proposes the toilet cleaning to the user when the estimated number of days has elapsed from the time point when the previous toilet cleaning is executed. Specifically, the agent proposes toilet cleaning, which is an action the user can take, to the user by causing the robot 100 to reproduce a voice saying “Are you going to clean the toilet?” or “The cleaning time of the toilet may be getting closer”. The agent may display these messages on the screen of the electronic equipment, instead of reproducing the voices.

(4) In a case in which a child at home performs personal grooming to go out, the agent monitors the action of the child to record the action of personal grooming in the past and record the timing at which the child performs personal grooming (time point when personal grooming is started, time point when personal grooming is finished, etc.). The agent records the actions of past personal grooming a plurality of times to estimate the timing of performing personal grooming by the child (for example, in the case of a weekday, near the time to go out to school, and in the case of a holiday, near the time to go out to take classes) based on the timing of performing personal grooming for each person who has performed personal grooming. In this way, the agent may estimate the next execution timing of the personal grooming by recording the personal grooming execution timing, and proposes that the user start the personal grooming at the estimated execution timing. Specifically, the agent proposes that the user start personal grooming, which is an action the user can take, by causing the robot 100 to reproduce a voice saying “It's time to go to the cram school” or “Don't you have morning practice today?”. The agent may display these messages on the screen of the electronic equipment, instead of reproducing the voices.

The agent may make a plurality of proposals to the user at specific intervals. Specifically, in a case in which the user does not take an action for the proposal even though the agent has made the proposal to the user, the agent may make the proposal to the user once or a plurality of times. As a result, since the user cannot immediately perform the specific action, the user can perform the specific action without forgetting the specific action even if the user holds the specific action for a while.

The agent may give a notification of a specific action in advance a certain period of time before a time point at which the estimated number of days has elapsed. For example, in a case in which the next watering execution timing is a specific day after passage of 20 days from the time point at which the previous watering was executed, the agent may give a notification for encouraging the next watering several days before the specific day. Specifically, the agent causes the robot 100 to reproduce a voice saying “The time to water plants is approaching”, “It is about time to water plants”, or the like so that the user can grasp the watering execution timing.

As described above, according to the action control system of the disclosure, the electronic equipment such as the robot 100 or the smartphone installed in a home stores all actions of the family members of the user of the electronic equipment, and can spontaneously propose any action at an appropriate timing, such as at which timing the user should clip the nail, whether the user should water plants, whether the user should clean the toilet, or whether the user should start personal grooming.

The action determination unit 236 spontaneously executes, as a robot action, reproducing, as audio, the action content of “(11)” described above, in other words, a proposal for encouraging the user in the home to take an action that the user can take.

The action determination unit 236 can spontaneously execute, as a robot action, displaying, on a screen, a message of the action content of “(12)” described above, in other words, a proposal for encouraging the user in the home to take an action that the user can take.

The memory control unit 238 may store, in the history data 222, information obtained by monitoring the user with respect to the action content of “(11)” described above, specifically, as examples of actions executed by the user at home, housework, nail clipping, watering plants, personal grooming to go out, walking the pet, and the like. The memory control unit 238 may store these pieces of information regarding the types of actions as specific information associated with the timings at which the actions are performed.

The memory control unit 238 may store, in the history data 222, information obtained by monitoring the user with respect to the action content of “(11)” described above, specifically, as examples of actions executed by the user at home, cleaning the toilet, meal preparation, cleaning the bathtub, taking in the laundry, floor cleaning, childcare, shopping, taking out the trash, room ventilation, and the like. The memory control unit 238 may store these pieces of information regarding the types of actions as specific information associated with the timings at which the actions are performed.

The memory control unit 238 may store, in the history data 222, information obtained by monitoring the user with respect to the action content of “(12)” described above, specifically, as examples of actions executed by the user at home, housework, nail clipping, watering plants, personal grooming to go out, walking the pet, and the like. The memory control unit 238 may store these pieces of information regarding the types of actions as specific information associated with the timings at which the actions are performed.

The memory control unit 238 may store, in the history data 222, information obtained by monitoring the user with respect to the action content of “(12)” described above, specifically, as examples of actions executed by the user at home, cleaning the toilet, meal preparation, cleaning the bathtub, taking in the laundry, floor cleaning, childcare, shopping, taking out the trash, room ventilation, and the like. The memory control unit 238 may store these pieces of information regarding the types of actions as specific information associated with the timings at which the actions are performed.

The action control unit 250 may cause the avatar to be displayed in the image display area of the electronic equipment or to operate according to the action determined by the action determination unit 236.

In particular, in a case in which the action determination unit 236 spontaneously or periodically determines, as an avatar action, a proposal for encouraging the user in the home to take an action based on the history data, the action determination unit may cause the action control unit 250 to operate the avatar so as to follow the proposal for encouraging the user to take the action at a timing at which the user should perform the action. The action content will be specifically described below.

It may be interpreted that the action determination unit 236 spontaneously acquires a state of the user proactively without a trigger from outside.

The trigger from outside may include a question from the user to the action determination unit 236, the avatar, or the like, an active action from the user to the action determination unit 236, the avatar, or the like. The term “periodically” may be interpreted as a specific cycle such as in units of one second, one minute, one hour, several hours, several days, week, or day of the week.

The actions performed by the user at home may include housework, nail clipping, watering plants, personal grooming to go out, walking pets, and the like. The housework may include cleaning the toilet, preparing a meal, cleaning the bath, taking in laundry, cleaning the floor, childcare, shopping, emptying the trash can, ventilating a room, and the like.

In the autonomous processing, the memory control unit 238 may store the type of action executed by the user at home as history data in association with the timing at which the action is performed. Specifically, the memory control unit 238 may store user information of a user (person) included in a specific family, information indicating the type of action such as housework performed by the user at home, and a past timing at which each of the actions was performed in association with each other. The past timing may be the number of times of execution of at least one or more actions.

In the autonomous processing, in a case in which the action determination unit 236 spontaneously or periodically determines, as an action of the avatar, based on the history data of the memory control unit 238, a proposal for encouraging the user in the home to take an action, the action determination unit may cause the action control unit 250 to operate the avatar so as to execute the proposal for encouraging the user to take the action at a timing at which the user should perform the action.

Hereinafter, examples of contents to be proposed to the user will be described.

(1) In a case in which the husband at home performs nail clipping, the state recognition unit 230 monitors the action of the husband, so that the memory control unit 238 records the past nail clipping operation and records the timing at which the nail clipping was performed (a time point at which the nail clipping was started, a time point at which the nail clipping was finished, and the like). Since the memory control unit 238 records the past nail-cutting operation a plurality of times, the action determination unit 236 estimates the nail-cutting interval (for example, the number of days such as 10 days and 20 days) of the husband based on the timing at which nail-cutting was performed for each person who has performed nail-cutting. In this manner, by recording the execution timing of the nail clipping, the action determination unit 236 may estimate the execution timing of next nail clipping, and may propose nail clipping to the user through the operation of the avatar by the action control unit 250 when the estimated number of days has elapsed from the time when the previous nail clipping was performed. Specifically, the action determination unit 236 may propose nail clipping, which is an action the user can take, to the user by reproducing a voice saying “Would you like to clip a nail now?” “Your nails might be getting long” or the like as the action of the avatar by the action control unit 250 at the time point when 10 days have elapsed from the previous nail clipping. The action determination unit 236 may cause the avatar to display images corresponding to these messages in the image display area as an action of the avatar by the action control unit 250, instead of reproducing such voice. For example, the avatar having an animal appearance may be transformed into a text message, or balloon text corresponding to the message may be displayed near the mouth of the avatar.

(2) In a case in which the wife at home performs watering plants, the state recognition unit 230 monitors the action of the wife, so that the memory control unit 238 records the past watering operation and records the timing at which the watering was performed (a time point at which the watering was started, a time point at which the watering was finished, and the like). Since the memory control unit 238 records the past watering operation a plurality of times, the action determination unit 236 estimates a watering interval (for example, the number of days such as 10 days and 20 days) of the wife based on the timing at which watering was performed for each person who has watered. In this manner, by recording the execution timing of watering, the action determination unit 236 may estimate the execution timing of next watering, and may propose the execution timing to the user when the estimated number of days has elapsed from the time point when the previous watering was performed. Specifically, the action determination unit 236 may propose watering, which is an action the user can take, to the user by reproducing a voice saying “Would you like to water now?” “The amount of water in plants might be getting low” or the like as the action of the avatar by the action control unit 250. The action determination unit 236 may cause the avatar to display images corresponding to these messages in the image display area as an action of the avatar by the action control unit 250, instead of reproducing such voice. For example, the avatar having an animal appearance may be transformed into a text message, or balloon text corresponding to the message may be displayed near the mouth of the avatar.

(3) In a case in which a child at home cleans a toilet, the state recognition unit 230 monitors an action of the child, and thereby, the memory control unit 238 records the past toilet cleaning operation and records the timing at which the toilet cleaning was performed (the time point at which the toilet cleaning was started, the time point at which the toilet cleaning was finished, and the like). Since the memory control unit 238 records the past toilet cleaning operation a plurality of times, the action determination unit 236 estimates an interval of the toilet cleaning by the child (for example, the number of days such as 7 days and 14 days) based on the timing at which toilet cleaning was performed for each person who cleaned the toilet. In this manner, by recording the execution timing of toilet cleaning, the action determination unit 236 may estimate the execution timing of next toilet cleaning, and may propose toilet cleaning to the user when the estimated number of days has elapsed from the time point when the previous toilet cleaning was performed. Specifically, the action determination unit 236 proposes toilet cleaning, which is an action the user can take, to the user by reproducing a voice saying “Are you going to clean the toilet?”, “The cleaning time of the toilet may be getting closer”, or the like as an action of the avatar by the action control unit 250. The action determination unit 236 may cause the avatar to display images corresponding to these messages in the image display area as an action of the avatar by the action control unit 250, instead of reproducing such voice. For example, the avatar having an animal appearance may be transformed into a text message, or balloon text corresponding to the message may be displayed near the mouth of the avatar.

(4) In a case in which a child at home performs personal grooming to go out, the state recognition unit 230 monitors the action of the child, so that the memory control unit 238 records the past personal grooming and records the timing at which the personal grooming was performed (a time point at which the personal grooming was started, a time point at which the personal grooming was finished, and the like). Since the memory control unit 238 records the past personal grooming operation a plurality of times, the action determination unit 236 estimates the timing at which the child prepares (for example, around the time of leaving for school on weekdays, and around the time of leaving for extracurricular lessons on holidays) based on the timing of performing personal grooming for each person who has performed personal grooming. In this way, by recording the performance timing of personal grooming, the action determination unit 236 may estimate the execution timing of the next personal grooming, and propose to the user that the user should start personal grooming at the estimated execution timing. Specifically, the action determination unit 236 may propose to the user that the user should start personal grooming, which is an action the user can take, by reproducing a voice saying “It's time to go to the cram school” “Isn't today morning practice day?” or the like as the action of the avatar by the action control unit 250. The action determination unit 236 may cause the avatar to display images corresponding to these messages in the image display area as an action of the avatar by the action control unit 250, instead of reproducing such voice. For example, the avatar having an animal appearance may be transformed into a text message, or balloon text corresponding to the message may be displayed near the mouth of the avatar.

The action determination unit 236 may execute a proposal to the user as an action of the avatar by the action control unit 250 a plurality of times at specific intervals. Specifically, in a case in which the user does not take the action related to the proposal even though the proposal has been made to the user, the action determination unit 236 may make the proposal to the user once or a plurality of times as actions of the avatar by the action control unit 250. As a result, since the user cannot immediately perform the specific action, the user can perform the specific action without forgetting the specific action even if the user holds the specific action for a while. Note that, in a case in which the user does not take the action related to the proposal, the avatar with a specific appearance may be transformed into a shape other than the specific appearance. Specifically, the avatar with a human appearance may be transformed into an avatar with a beast appearance. Furthermore, in a case in which the user does not take the action related to the proposal, the voice reproduced from the avatar may change from a specific tone to a tone other than the specific tone. Specifically, the voice emitted from the avatar with the human appearance may change from a gentle tone to a rough tone.

The action determination unit 236 may notify in advance the user of the specific action as an action of the avatar by the action control unit 250, a certain period of time before the time point at which the estimated number of days has elapsed. For example, in a case in which the next watering execution timing is a specific day after 20 days elapse from the time point at which the previous watering is executed, the action determination unit 236 may execute a notification encouraging the next watering several days before the specific day as the action of the avatar by the action control unit 250. Specifically, the action determination unit 236 causes a voice saying “The time to water plants is approaching”, “It is about time to water plants”, or the like to be reproduced as an action of the avatar by the action control unit 250 so that the user can ascertain the watering execution timing.

As described above, according to the action control system of the disclosure, the headset-type terminal installed in a home stores all actions of the family members of the user who use the headset-type terminal, and can spontaneously propose, as an action of the avatar, any action at an appropriate timing, such as at which timing the user should clip the nails, whether the user should water plants, whether the user should clean the toilet, or whether the user should start personal grooming.

### Twenty-Fifth Embodiment

In this embodiment, it is preferable that the action determination unit 236 determine the content of an utterance or a gesture and cause the action control unit to control the avatar so as to provide learning support to the user 10 based on sensory characteristics of the user 10.

Specifically, the action determination unit 236 inputs data indicating at least one of a state of the user 10, a state of electronic equipment, an emotion of the user 10, or an emotion of an avatar, together with data for asking about an avatar action to a data generation model, and determines an action of the avatar based on an output of the data generation model. At this time, the action determination unit 236 determines the content of an utterance or a gesture and causes the action control unit 250 to control the avatar so as to provide learning support to the user 10 based on sensory characteristics of the user 10.

In the embodiment, for example, a child having a developmental disorder is employed as the user 10. Furthermore, in the embodiment, the proprioceptive sense and vestibular sense are applied as senses in addition to the five senses (specifically, the sense of taste, smell, vision, hearing, and touch). The proprioceptive sense is a sense of one's own position, movement, and the degree of force applied. The vestibular sense is a sense of one's own inclination, speed, and rotation.

The electronic equipment (for example, the headset-type terminal 820) executes processing of assisting learning of the user based on the sensory characteristics of the user according to the following steps 1 to 5-2. Note that the robot 100 may execute processing of assisting learning of the user based on the sensory characteristics of the user according to the following steps 1 to 5-2.

(Step 1) The electronic equipment acquires a state of the user 10, an emotion value of the user 10, an emotion value of the avatar, and the history data 222.

Specifically, processing similar to steps S100 to S103 is performed to acquire the state of the user 10, the emotion value of the user 10, the emotion value of the avatar, and the history data 222.

(Step 2) The electronic equipment acquires sensory characteristics of the user 10. For example, the electronic equipment acquires the characteristic of the user being poor at visual information processing.

Specifically, the action determination unit 236 acquires sensory characteristics of the user 10 based on results of voice recognition, voice synthesis, expression recognition, motion recognition, self-position estimation, and the like by the sensor module unit 210. Note that the action determination unit 236 may acquire sensory characteristics of the user 10 from an occupational therapist in charge of the user 10, a parent or teacher of the user 10, or the like.

(Step 3) The electronic equipment determines a problem that the avatar presents to the user 10. Note that the problem according to the embodiment is a problem for training the senses related to the acquired characteristics.

Specifically, the action determination unit 236 adds a fixed sentence “At this time, what is a question recommended to the user?” to the text representing the characteristics of the senses of the user 10, the emotion of the user 10, the emotion of the avatar, and the content stored in the history data 222, inputs the text to the sentence generation model, and acquires a question to be recommended. At this time, a question suitable for the user 10 can be presented by considering not only the sensory characteristics of the user 10 but also the emotion of the user 10 and the history data 222. In addition, by considering the emotion of the avatar, it is possible to make the user 10 feel that the avatar has emotions. However, the present invention is not limited to this example. Without considering the emotion of the user 10 or the history data 222, the action determination unit 236 may add a fixed sentence “At this time, what is a question recommended to the user?” to the text indicating the sensory characteristics of the user 10, input the text to the sentence generation model, and acquire a question to be recommended.

(Step 4) The electronic equipment presents the question determined in step 3 to the user 10, and acquires an answer of the user 10.

Specifically, the action determination unit 236 determines an utterance to present a question to the user 10 as an action of the avatar, and the action control unit 250 controls the control target 252 and makes an utterance to present the question to the user 10. The state recognition unit 230 recognizes the state of the user 10 based on the information analyzed by the sensor module unit 210, and the emotion determination unit 232 determines an emotion value indicating the emotion of the user 10 based on the information analyzed by the sensor module unit 210 and the state of the user 10 recognized by the state recognition unit 230.

The action determination unit 236 determines whether the reaction of the user 10 is positive based on the state of the user 10 recognized by the state recognition unit 230 and the emotion value indicating the emotion of the user 10, and determines whether to execute processing of raising the difficulty level of the question, change the type of the question, or lower the difficulty level as an action of the avatar. Here, the case of the reaction of the user 10 being positive includes the case of the answer of the user 10 being correct. However, even if the answer of the user 10 is correct, in a case in which the user 10 is in an “unpleasant” state, the action determination unit 236 may determine that the reaction of the user 10 is not positive.

Note that the action determination unit 236 may determine an utterance content to support the user 10 (for example, “do your best”, “You don't need to rush, just do it slowly.”, or the like) based on the state of the user 10 recognized by the state recognition unit 230 and the emotion value indicating the emotion of the user 10 until the answer of the user 10 is acquired, and the action control unit 250 may cause the avatar to make an utterance. Note that, in a case in which the action determination unit 236 determines a content supporting the user 10, the display mode of the avatar may be changed to an avatar in a predetermined display mode (for example, the look of a cheerleading group, a cheer leader, or the like), and the action control unit 250 may transform the avatar and make the avatar utter.

(Step 5-1) In a case in which the reaction of the user 10 is positive, the electronic equipment executes processing of raising the difficulty level of the question presented.

Specifically, in a case in which it is determined to present a question with an increased difficulty level to the user 10 as an action of the avatar, the action determination unit 236 adds a fixed sentence “Is there a more difficult problem?” to the text indicating the sensory characteristics of the user 10, the emotion of the user 10, the emotion of the avatar, and the content stored in the history data 222, and inputs the text to the sentence generation model, thereby acquiring a question with a higher difficulty level. Then, the processing returns to step 4 described above, and the processing of steps 4 to 5-2 described above is repeated until a predetermined time elapses.

(Step 5-2) In a case in which the reaction of the user 10 is not positive, the electronic equipment determines another type of question to be presented to the user 10 or a question with a lowered difficulty level. Here, another type of question is, for example, a question for training a sense different from the sense related to the acquired characteristics.

Specifically, in a case in which it is determined to present a question of a different type or a question with a lowered difficulty level to the user 10 as an action of the avatar, the action determination unit 236 adds a fixed sentence “Is there another question to be recommended to the user?” to the text indicating the sensory characteristics of the user 10, the emotion of the user 10, the emotion of the avatar, and the content stored in the history data 222, and inputs the text to the sentence generation model, thereby acquiring a question to be recommended. Then, the processing returns to step 4 described above, and the processing of steps 4 to 5-2 described above is repeated until a predetermined time elapses.

Note that the type and difficulty level of the question that the avatar presents may be changed. Furthermore, the action determination unit 236 may record the answer status of the user 10 so that an occupational therapist in charge of the user 10, or a parent, a teacher, or the like of the user 10 can view the answer status.

In this manner, the electronic equipment can provide learning support based on the sensory characteristics of the user.

### Twenty-Sixth Embodiment

In the embodiment, it is assumed that the user 10 attends an event and is in a situation of wearing the headset-type terminal 820 at the event venue.

In addition, the action control unit 250 displays the avatar in the image display area of the headset-type terminal 820 as the control target 252C according to the determined action of the avatar. Furthermore, in a case in which the determined action of the avatar includes the utterance content of the avatar, the utterance content of the avatar is output from the speaker as the control target 252C by voice. Note that, in the image display area of the headset-type terminal 820, a state of the event venue similar to that actually viewed by the user 10 without the headset-type terminal 820, that is, a state of the real world, is displayed.

In particular, in the embodiment, environment information of the event venue is acquired by the sensor unit 200B while the state of the event venue is displayed on the headset-type terminal 820 together with the avatar as described above. For example, the environment information includes the atmosphere of the event venue and an application of the avatar in the event. As the atmosphere, the information of atmosphere is a numerical value representing a quiet atmosphere, a bright atmosphere, a dark atmosphere, or the like. Examples of the application of the avatar include an event promoter, an event guide, and the like. The action determination unit 236 adds a fixed sentence “What are the lyrics and melodies that match the current atmosphere?” to the text indicating the information on the environment and inputs the text to the sentence generation model, and acquires lyrics and music scores of the melodies to be recommended regarding the environment of the event venue.

Here, the agent system 800 includes a sound synthesis engine. The action determination unit 236 inputs the lyrics and music scores of the melodies acquired from the sentence generation model to the sound synthesis engine, and acquires music based on the lyrics and melodies acquired from the sentence generation model. Further, the action determination unit 236 determines an avatar action content in which the avatar plays, sings, and/or dances to the acquired music.

The action control unit 250 generates an image in which the avatar is performing or singing the music acquired by the action determination unit 236 on a stage in a virtual space or dancing to the music. As a result, in the headset-type terminal 820, a state in which the avatar is performing, singing, or dancing to the music is displayed in the image display area.

As a result, the avatar can improvise music according to the atmosphere of the event venue, the role of the avatar, and the like displayed on the headset-type terminal 820, sing, or dance to the music, so the atmosphere of the event venue can be improved.

At this time, the action control unit 250 may change the expression of the avatar or change the movement of the avatar according to the content of the music. For example, in a case in which the content of the music is a pleasant content, the expression of the avatar may be changed to an expression of pleasure, or the movement of the avatar may be changed as if the avatar is dancing with fun choreography. Furthermore, the action control unit 250 may transform the avatar in accordance with the content of the music. For example, the action control unit 250 may transform the avatar into a form of a musical instrument of music to be played, or may transform the avatar to a form of a musical note.

### Twenty-Seventh Embodiment

In a case in which it is determined to answer to a question of the user 10 as an action corresponding to an action of the user 10, the action determination unit 236 acquires a vector (for example, an embedding vector) representing the content of the question of the user 10, searches a database (for example, a database included in a cloud server) storing combinations of questions and answers for a question having a vector corresponding to the acquired vector, and generates an answer to the question of the user using the answer to the searched question and a sentence generation model having an interaction function.

Specifically, the cloud server stores all data (conversation contents, texts, images, etc.) obtained from the past conversations, and the database stores combinations of questions and answers obtained from the data. The embedding vector representing the content of the question of the user 10 is compared with the embedding vector representing the content of each question in the database, and an answer to the question having the content closest to the content of the question of the user 10 is acquired from the database. In the embodiment, instead of acquiring the answer to the content of a question hit by keyword search, a question having the closest content is searched using an embedding vector obtained using a neural network, and the answer to the searched question is acquired. Then, by inputting the answer to the sentence generation model, an answer that makes a more realistic conversation can be obtained, and the answer can be uttered as an answer of the robot 100.

For example, it is assumed that, to a question “When is this product most sold?” of the user 10, an answer “This product is sold well during midsummer afternoon.” is acquired from the database. At this time, a generative AI, which is a sentence generation model, receives an input of “When someone asks ““When is this product most sold?”, and I want to answer with a sentence “This product is sold well during midsummer afternoon.”, what is the best response to that?”.

Note that all the combinations of the questions and the answers included in the manual of a call center may be stored in a database, an answer having the closest vector to the content of the question of the user 10 may be acquired from the database, and the answer of the robot 100 may be generated using the generative AI which is a sentence generation model. As a result, a conversation that most prevents cancellation is also established. Furthermore, a combination of an utterance of the user 10 side and an utterance of the robot 100 side may be stored in the database as a combination of a question and an answer, an answer having the closest vector to the content of the question of the user 10 may be acquired from the database, and an answer of the robot 100 may be generated using the generative AI that is a sentence generation model.

As in the first embodiment, the emotion determination unit 232 of the control unit 228B determines an emotion value of the agent based on the state of the headset-type terminal 820, and substitutes the emotion value as an emotion value of the avatar.

When the avatar performs a response process of responding to an action of the user 10 as in the first embodiment, the action determination unit 236 of the control unit 228B determines an action of the avatar based on at least one of a user state, a state of the headset-type terminal 820, an emotion of the user, or an emotion of the avatar.

In a case in which it is determined to answer to a question of the user 10 as an action of the avatar corresponding to an action of the user 10, the action determination unit 236 acquires a vector (for example, an embedding vector) representing the content of the question of the user 10, searches a database (for example, a database included in a cloud server) storing combinations of questions and answers for a question having a vector corresponding to the acquired vector, and generates an answer to the question of the user using the answer to the searched question and a sentence generation model having an interaction function.

Specifically, the cloud server stores all data (conversation contents, texts, images, etc.) obtained from the past conversations, and the database stores combinations of questions and answers obtained from the data. The embedding vector representing the content of the question of the user 10 is compared with the embedding vector representing the content of each question in the database, and an answer to the question having the content closest to the content of the question of the user 10 is acquired from the database. In the embodiment, instead of acquiring the answer to the content of a question hit by keyword search, a question having the closest content is searched using an embedding vector obtained using a neural network, and the answer to the searched question is acquired. Then, by inputting the answer to the sentence generation model, it is possible to obtain an answer that makes a more realistic conversation, and to utter the answer as an avatar answer.

For example, it is assumed that, to a question “When is this product most sold?” of the user 10, an answer “This product is sold well during midsummer afternoon.” is acquired from the database. At this time, the generative AI, which is a sentence generation model, receives an input of “When someone asks ““When is this product most sold?”, and I want to answer with a sentence “This product is sold well during midsummer afternoon.”, what is the best response to that?”.

Note that all the combinations of the questions and the answers included in the manual of a call center may be stored in a database, an answer having the closest vector to the content of the question of the user 10 may be acquired from the database, and the answer of the avatar may be generated using the generative AI which is a sentence generation model. As a result, a conversation that most prevents cancellation is also established. Furthermore, a combination of an utterance of the user 10 side and an utterance of the avatar side may be stored in the database as a combination of a question and an answer, an answer having the closest vector to the content of the question of the user 10 may be acquired from the database, and an answer of the avatar may be generated using the generative AI that is a sentence generation model.

As in the first embodiment, when an agent functioning as an avatar performs an autonomous process of autonomously acting, the action determination unit 236 of the control unit 228B determines, as an action of the avatar, any of multiple types of avatar actions including not acting, using at least one of the state of the user 10, the emotion of the user 10, the emotion of the avatar, or the state of electronic equipment (for example, the headset-type terminal 820) that controls the avatar, and the action determination model 221, at a predetermined timing.

Specifically, the action determination unit 236 inputs a text representing at least one of the state of the user 10, the state of the electronic equipment, the emotion of the user 10, or the emotion of the avatar, together with a text for inquiry about the action of the avatar to the sentence generation model, and determines the action of the avatar based on the output of the sentence generation model.

In addition, the action control unit 250 displays the avatar in the image display area of the headset-type terminal 820 as the control target 252C according to the determined action of the avatar. Furthermore, in a case in which the determined action of the avatar includes the utterance content of the avatar, the utterance content of the avatar is output from the speaker as the control target 252C by voice.

In a case in which it is determined to answer to a question of the user as an action of the avatar, the action control unit 250 may operate the avatar with a look corresponding to the question or the answer. For example, in a case of answering a question regarding a product, the avatar outfit is changed to a store clerk's style outfit to make the avatar operate.

### Twenty-Eighth Embodiment

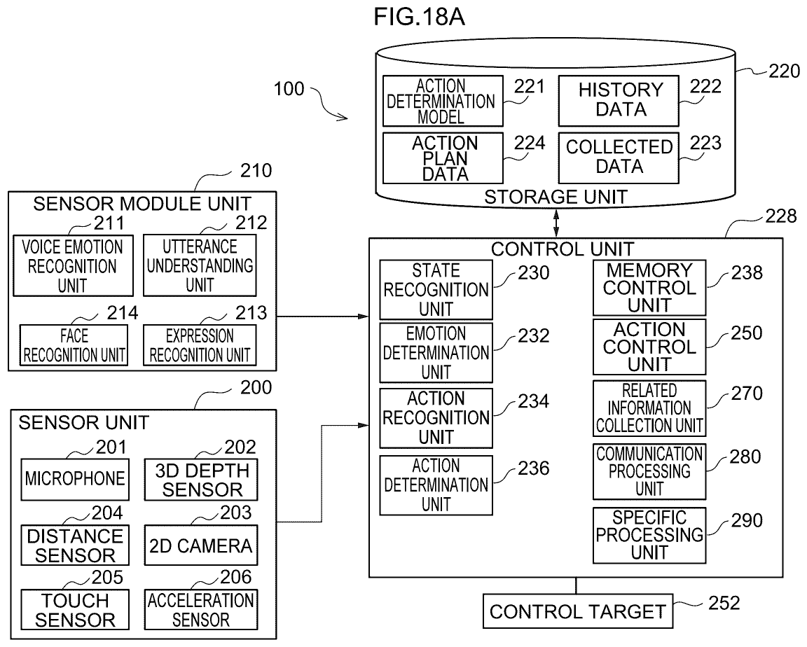

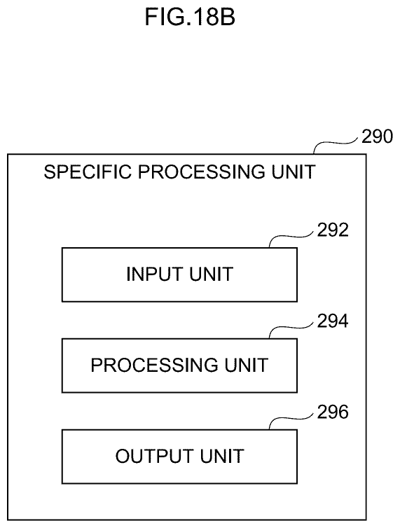

FIG. 18A schematically illustrates another functional configuration of the robot 100. The robot 100 further includes a specific processing unit 290.

In the autonomous processing in the embodiment, the robot 100 as an agent acquires information about baseball pitchers necessary for the user 10 from external data (web sites such as news sites and moving image sites, distribution news, and the like). The robot 100 autonomously acquires the information at all times even when the user 10 is absent, that is, even when the user 10 is not around the robot 100. Then, when the robot 100 as an agent detects that the user 10 requests provision of pitch information regarding the next pitch of a specific pitcher to be described later, the robot 100 provides the pitch information regarding the next pitch of the specific pitcher.

For example, multiple types of the robot actions include the following (1) to (11).
(1) The robot does nothing.
(2) The robot dreams.
(3) The robot speaks to the user.
(4) The robot creates a picture diary.
(5) The robot proposes an activity.
(6) The robot proposes a person whom the user should meet.
(7) The robot introduces news that the user is interested in.
(8) The robot edits pictures and videos.
(9) The robot studies with the user.
(10) The robot evokes a memory.
(11) The robot provides pitch information to the user.

Furthermore, for example, event data having a large emotion value of the robot 100 is selected as an impressive memory of the robot 100. This makes it possible to create an emotion change event based on the event data selected as an impressive memory.

In a case in which the action determination unit 236 determines, as a robot action, “(11) Provides pitch information to the user.”, that is, to provide the user with pitch information regarding the next pitch of a specific baseball pitcher, the action determination unit provides the user with the pitch information.

Based on the state of the user 10 recognized by the state recognition unit 230, in a case in which an action of the user 10 with respect to the robot 100 is detected in a state where there is no action of the user 10 with respect to the robot 100, the action determination unit 236 reads data stored in the action plan data 224 and determines an action of the robot 100.

For example, in a case in which the user 10 is absent around the robot 100 but the user 10 is detected, the action determination unit 236 reads data stored in the action plan data 224 and determines an action of the robot 100. In addition, when it is detected that the user 10 has woken up in a case in which the user 10 was sleeping, the action determination unit 236 reads data stored in the action plan data 224 and determines an action of the robot 100.

Next, a specific process in a case in which the robot 100 determines “(11) Provide pitch information to the user.” as a robot action will be described. The specific process is a process of the specific processing unit 290 when a process of creating pitch information regarding the next pitch of a specific pitcher is performed as the specific process in a case in which there is an input from the user. Note that the robot 10 may determine “(11) Provide pitch information to the user.” as a robot action without any input from the user. In other words, the “(11) Provide pitch information to the user.” may be autonomously determined based on the state of the user 10 recognized by the state recognition unit 230.

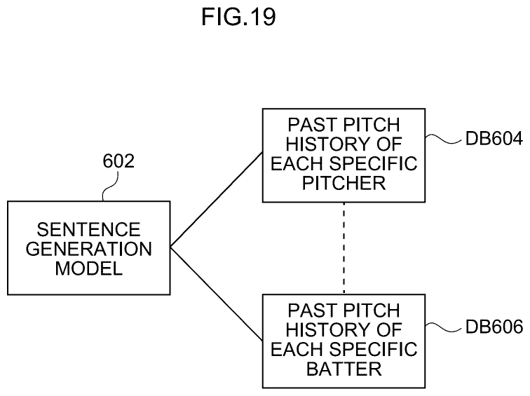

In the specific process in the embodiment, as illustrated in FIG. 19, the sentence generation model 602 used to create the pitch information is connected to a past pitch history DB of each specific pitcher 604 and a past pitch history DB of each specific batter 606. The past pitch history DB of each specific pitcher 604 stores a past pitch history associated with each registered specific pitcher. Specific examples of the content stored in the past pitch history DB of each specific pitcher 604 include pitching days, the number of pitches, pitch types, pitch course, opposing batters, results (hits, strikeouts, home runs, and the like), and the like. The past pitch history DB of each specific batter 606 stores a past pitch history associated with each registered specific batter. Specific examples of the content stored in the past pitch history DB of each specific batter 606 include pitching days, the number of pitches, pitch types, pitch course, opposing batters, results (hits, strikeouts, home runs, and the like), and the like. The specific sentence generation model 602 is subjected to fine tuning in advance to additionally learn each piece of information stored in the DBs 604 and 606.

As illustrated in FIG. 18B, the specific processing unit 290 includes an input unit 292, a processing unit 294, and an output unit 296.

The input unit 292 receives a user input. Specifically, audio input of the user, text input via a mobile terminal, or the like is acquired. For example, the user inputs a text or a voice requesting the pitch information of the next pitch of the specific pitcher, such as “Tell me information regarding the next pitch of the specific pitcher [Name]”.

The processing unit 294 determines whether a predetermined trigger condition is satisfied. For example, the trigger condition is that a text or a voice requesting pitch information of the next pitch of a specific pitcher, such as “Tell me information of the next pitch of the specific pitcher [Name]”, has been accepted.

Note that the processing unit 294 may optionally cause the user to input batter information of the opponent team if the trigger condition is satisfied. The batter information may be a specific batter (batter name) or may be simply a distinction between a left-handed batter and a right-handed batter.

Then, the processing unit 294 inputs a text indicating an instruction for obtaining data for the specific process to the sentence generation model, and acquires the processing result based on the output of the sentence generation model. More specifically, as the specific process, the processing unit 294 generates a sentence (prompt) that instructs creation of the pitch information of the next pitch of the specific pitcher based on a request accepted by the input unit 292, and performs processing of inputting the generated sentence to the sentence generation model 602, and acquires the pitch information of the next pitch of the specific pitcher. For example, the processing unit 294 generates a prompt “Please create pitch information of the next pitch of the specific pitcher [Name] against opposing batter [Name] with a count of 2 balls, 1 strike, and 2 outs.”. The pitch information includes pitch types and ball trajectories (distinction between outside, inside, high, and low). Then, the processing unit 294 acquires, for example, an answer such as “Specific Pitcher [Name], the next pitch seems likely to be outside, low, and a fastball” from the sentence generation model 602.

Note that the processing unit 294 may perform the specific process using a state of the user or a state of the robot 100, and the sentence generation model. In addition, the processing unit 294 may perform the specific process using an emotion of the user or an emotion of the robot 100, and the sentence generation model.

The output unit 296 controls actions of the robot 100 so as to output results of the specific process. Specifically, pitch information of a ball that a specific pitcher throws next is displayed on a display device provided in the robot 100, uttered by the robot 100, or transmitted in a message to the user of a message application of the user's mobile terminal.

Note that a part of the robot 100 (for example, the sensor module unit 210, the storage unit 220, and the control unit 228) may be provided outside the robot 100 (for example, on a server), and the robot 100 may function as each unit of the robot 100 by communicating with the outside.

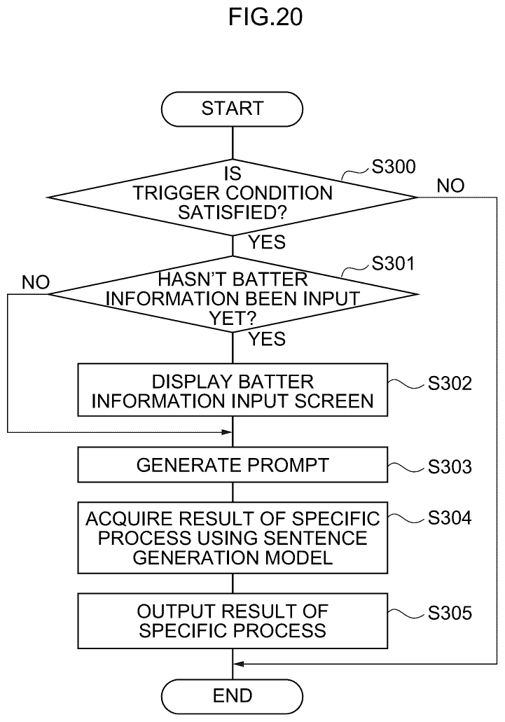

FIG. 20 schematically shows an example of an operation flow related to an operation of the robot 100 in a specific process to create pitch information of the next pitch of a specific pitcher. The operation flow shown in FIG. 20 is repeatedly and automatically executed, for example, each time a certain time elapses.

In step S300, the processing unit 294 determines whether a predetermined trigger condition is satisfied. For example, the processing unit 294 determines whether information indicating a request for creation of pitch information of the next pitch of a specific pitcher, such as “Tell me the information of the next pitch of the specific pitcher [Name]”, has been input from the user 10. If the trigger condition is satisfied, the processing proceeds to step S301. On the other hand, if the trigger condition is not satisfied, the specific process ends.

In step S301, the processing unit 294 determines whether the opposing batter information has not been input by the user, and if not, displays an input screen to be input by the user on a display device provided in the robot 100 in step S302, and requests the user to input the opposing batter information. In a case in which the opposing batter information has been input from the user, the processing proceeds to step S303.

In a case in which the batter information has been input by the user or there is no input for a predetermined time, the processing proceeds to step S303, and the processing unit 294 adds an instruction sentence for obtaining the result of the specific process to a text indicating an input and generates a prompt. For example, the processing unit 294 generates a prompt “Please create pitch information of the next pitch of the specific pitcher [Name] against opposing batter [Name] with a count of 2 balls, 1 strike, and 2 outs.”.

In step S304, the processing unit 294 inputs the generated prompt to the sentence generation model 602, and acquires the output of the sentence generation model 602, that is, the pitch information of the next pitch of the specific pitcher.

In step S305, the output unit 296 controls the action of the robot 100 so as to output the result of the specific process, and the specific process ends. In the output of the result of the specific process, for example, a text “Specific Pitcher [Name], the next pitch seems likely to be outside, low, and a fastball” is displayed.

Based on the pitch information, the batter against the specific pitcher [Name] can predict the next pitch and can be ready in the batter's box according to the pitch information.

### Twenty-Ninth Embodiment

In the specific process in the embodiment, for example, when the user 10 such as a producer or an announcer of a television station makes an inquiry for information regarding earthquakes, a text (prompt) based on the inquiry is generated, and the generated text is input to the sentence generation model. The sentence generation model generates information regarding earthquakes inquired by the user 10 based on the input text and various types of information such as information regarding past earthquakes in a designated region (including information of disasters caused by the earthquakes), weather information in the designated region, and information regarding terrain of the designated region. The generated information regarding earthquake is output as audio from a speaker mounted in the robot 100 to the user 10, for example. The sentence generation model can acquire various types of information from an external system using, for example, the ChatGPT plug-in. Examples of the external system include a system that provides map information of various regions, a system that provides weather information of various regions, a system that provides information regarding terrain of various regions, and a system that provides information regarding past earthquakes in various regions, and the like. Note that designation of a region can be performed by the name, address, location information, and the like of the region. The map information includes information of roads, rivers, seas, mountains, forests, residential areas, and the like of the designated region. The weather information includes wind directions, wind speeds, temperature, humidity, seasons, chances of precipitation, and the like of the designated region. The information regarding terrain includes inclination, undulation, and the like of the ground surface of the designated region.

As illustrated in FIG. 2B, the specific processing unit 290 includes the input unit 292, the processing unit 294, and the output unit 296.

The input unit 292 receives a user input. Specifically, the input unit 292 acquires text input and audio input of the user 10. As the information regarding earthquakes input by the user 10, for example, seismic intensity, magnitude, epicenter (place name or latitude/longitude), depth of epicenter, and the like are input.

The processing unit 294 performs the specific process using the sentence generation model. Specifically, the processing unit 294 determines whether a predetermined trigger condition is satisfied. More specifically, as the trigger condition, that the input unit 292 has accepted a user input for inquiring information regarding the earthquake (for example, “What measures should be taken for the region ABC against the recent earthquake?”) may be set.

Then, if the trigger condition is satisfied, the processing unit 294 inputs a text indicating an instruction for obtaining data for the specific process to the sentence generation model, and acquires the processing result based on the output of the sentence generation model. Specifically, the processing unit 294 acquires the result of the specific process using the output of the sentence generation model when a text from the user 10 instructing the presentation of the information regarding the earthquake is set as an input sentence. More specifically, the processing unit 294 generates a text in which the map information, the weather information, and the information regarding terrain provided from the system described above are added to the user input acquired by the input unit 292, thereby generating a text instructing presentation of the information regarding the earthquake in the region designated by the user 10. Then, the processing unit 294 inputs the generated text to the sentence generation model, and acquires information regarding the earthquake in the region designated by the user 10 based on the output of the sentence generation model. Note that the information regarding the earthquake in the region designated by the user 10 may be rephrased as information regarding the earthquake in the region inquired by the user 10.

The information regarding the earthquake may include information regarding past earthquakes in the region designated by the user 10. Examples of the information regarding past earthquakes in the designated region include the latest seismic intensity of the designated region, the maximum depth of the designated region in the past one year, and the number of earthquakes in the designated region in the past one year. In addition, the information regarding past earthquakes in the designated region may include information of disasters caused by the earthquake in the designated region. Further, information of disasters caused by earthquakes in regions having similar terrain to the designated region may be included. Here, examples of the information of disasters caused by earthquakes include sediment disasters (e.g., cliff collapses, landslides), tsunamis, and the like.

Note that the processing unit 294 may perform the specific process using a state of the user or a state of the robot 100, and the sentence generation model. In addition, the processing unit 294 may perform the specific process using an emotion of the user or an emotion of the robot 100, and the sentence generation model.

The output unit 296 controls actions of the robot 100 so as to output results of the specific process. Specifically, the output unit 296 displays the information regarding earthquakes on the display device provided in the robot 100, or the information is caused to be uttered by the robot 100 or transmitted in a message to the user 10 of a message application of the user's mobile terminal.

Note that a part of the robot 100 (for example, the sensor module unit 210, the storage unit 220, and the control unit 228) may be provided outside the robot 100 (for example, on a server), and the robot 100 may function as each unit of the robot 100 by communicating with the outside.

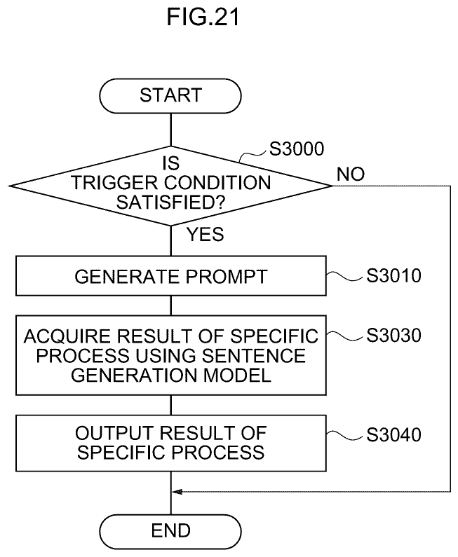

FIG. 21 schematically shows an example of an operation flow related to an operation in which the robot 100 performs a specific process of supporting the user 10 with announcement of information regarding earthquakes.

In step S3000, the processing unit 294 determines whether a predetermined trigger condition is satisfied. For example, in a case in which an input from the user 10 to inquire information regarding an earthquake (for example, “What measures should the region ABC take against the recent earthquake with magnitude D, epicenter EFG, and depth of epicenter H km?”) has been accepted by the input unit 292, the processing unit 294 determines that the trigger condition is satisfied.

If the trigger condition is satisfied, the processing proceeds to step S3010. On the other hand, if the trigger condition is not satisfied, the specific process ends.

In step S3010, the processing unit 294 adds map information, weather information, and information regarding terrain of the designated region to the text indicating user input, and generates a prompt. For example, using the user input “What measures should the region ABC take against the recent earthquake with magnitude D, epicenter EFG, and depth of epicenter H km?”, the processing unit 294 generates a prompt “Magnitude D, epicenter EFG, depth of epicenter H km, the season is winter; and in the designated region ABC, the seismic intensity is 4, the temperature is 1° C., it was rainy yesterday, it feels cold, there are many cliffs, and there are many areas with an elevation of J m. What earthquake countermeasures should local residents take at this time?”.

In step S3030, the processing unit 294 inputs the generated prompt to the sentence generation model, and acquires the result of the specific process based on the output of the sentence generation model. For example, the sentence generation model may acquire information regarding past earthquakes (including disaster information) in the region designated by the user 10 from the above-described external system based on the input prompt, and generate the information regarding the earthquake based on the acquired information.

For example, as an answer to the above prompt, the sentence generation model generates sentences indicating “There was an earthquake in the region ABC. Seismic intensity 4, epicenter EFG (longitude K degrees or latitude L degrees), and depth of epicenter H km. Since it rained yesterday, there is also a possibility of cliff collapse. Even in the earthquake one year ago, a rock collapse occurred along the national road, so the possibility of rock collapses is quite high. Furthermore, the coastal area of the region ABC has a low elevation, so an N-meter tsunami could arrive at the coastal area as early as M minutes from now. Even in the earthquake one year ago, the tsunami has arrived, so the local residents should prepare for evacuation”.

In step S3040, the output unit 296 controls actions of the robot 100 so as to output the result of the specific process as described above, and ends the specific process. In such a specific process, an announcement suitable for the region can be made for the earthquake. The viewer of the earthquake alert can easily take measures against earthquakes by the announcement suitable for the region.

In addition, the result of notifying the viewers of the earthquake alert on the information regarding earthquakes based on the sentence generation model using the generative AI and the actual damage situations with respect to the notification result may be used as input information and reference information when a new generative AI is used. When such information is used, the accuracy of the information when evacuation instructions are issued to local residents is improved.

Furthermore, a generative model is not limited to the sentence generation model that outputs (generates) results based on sentences, and a generative model that outputs (generates) results based on input of information such as images and sound may be used. For example, the generative model may output results based on images of seismic intensity, epicenter, depth of epicenter, and the like projected on a broadcast screen for earthquake alert, or may output results as sound of seismic intensity, epicenter, depth of epicenter, and the like from an announcer of the earthquake alert.

Although the system according to the disclosure has been described focusing on the functions of the robot 100, the system according to the disclosure is not necessarily implemented on a robot. The system according to the disclosure may be implemented as a general information processing system. The disclosure may be implemented as, for example, a software program that operates on a server or a personal computer, or an application that operates on a smartphone or the like. The method according to the invention may be provided to a user in a form of software as a Service (SaaS).

In another aspect of the embodiment, the following specific process is performed similarly to the above-described aspects. In the specific process, for example, when the user 10 such as a producer or an announcer of a television station makes an inquiry for information regarding an earthquake, a text (prompt) based on the inquiry is generated, and the generated text is input to the sentence generation model. The sentence generation model generates information regarding earthquakes inquired by the user 10 based on the input text and various types of information such as information regarding past earthquakes in a designated region (including information of disasters caused by the earthquakes), weather information in the designated region, and information regarding terrain of the designated region. The generated information regarding the earthquake is output as audio from the speaker to the user 10 as an utterance content of the avatar. The sentence generation model can acquire various types of information from an external system using, for example, the ChatGPT plug-in. As an example of the external system, the same system as that of the first embodiment may be used. Note that designation of a region, map information, weather information, information regarding terrain, and the like are also the same as those in the above-described aspects.

Also in another aspect, the specific processing unit 290 includes the input unit 292, the processing unit 294, and the output unit 296 as illustrated in FIG. 2B. The input unit 292, the processing unit 294, and the output unit 296 function and operate as those in the first embodiment. In particular, the processing unit 294 of the specific processing unit 290 performs a specific process using the sentence generation model, for example, processing similar to the example of the operation flow shown in FIG. 21.

In another aspect, the output unit 296 of the specific processing unit 290 controls actions of the avatar so as to output results of the specific process. Specifically, the avatar is caused to display or utter the information regarding the earthquake acquired by the processing unit 294 of the specific processing unit 290.

In another aspect, the action control unit 250 may change an action of the avatar according to a result of the specific process. For example, the intonation of the utterance of the avatar, the expression at the time of the utterance, gestures, and the like may be changed according to the result of the specific process. Specifically, in a case in which the information regarding an earthquake has urgency, the intonation of an utterance of the avatar may be increased so that the user 10 can easily recognize important matters, the display at the time of the utterance of the avatar may have a serious expression so that the user 10 can easily recognize that the important matters are being uttered, or the user 10 can easily recognize from gestures of the avatar that the important matters are being uttered. Such an action (announcement) of the avatar makes it easier for the viewers to understand the situations of the earthquake and to take measures against the earthquake.

Furthermore, when controlling the avatar to utter information regarding an earthquake, the action control unit 250 may change the appearance of the avatar to an announcer, a newscaster, or the like that reports news.

In a case in which an action of the user 10 with respect to the avatar is detected from a state in which there is no action of the user 10 with respect to the avatar based on the state of the user 10 recognized by the state recognition unit 230, the action determination unit 236 reads data stored in the action plan data 224 and determines an action of the avatar.

### Thirtieth Embodiment

In this embodiment, the action determination unit 236 analyzes a social networking service (social media) related to the user by using the sentence generation model, and recognizes matters that the user is interested in based on results of the analysis. Examples of the social media related to the user include social media that the user usually browses or the user's own social media. In this case, the action determination unit 236 acquires information regarding a spot and/or an event to be recommended to the user at the user's current position, and determines an action of the avatar to propose the acquired information to the user. Note that, in a case in which the user travels to a location with which they are entirely unfamiliar, the user's convenience can be achieved by proposing a spot and/or an event to be recommended to the user. Furthermore, when the user selects a plurality of spots and/or a plurality of events in advance, the action determination unit 236 may determine the most efficient route for going around the plurality of spots and/or the plurality of events in consideration of the congestion status of the day and the like, and provide the information to the user.

The action control unit 250 controls the avatar such that the avatar proposes to the user information that the action determination unit 236 proposes to the user. In this case, the action control unit 250 displays the state of the real world on the headset-type terminal 820 together with the avatar, and operates the avatar to guide the user to the spot and/or the event. Specifically, the avatar is operated to utter guidance to the spot and/or the event, or to have a panel in which an image or text for guiding the user to the spot and/or the event is described. The guidance content may include not only the selected spot and/or event, but also guidance content similar to what a human tour guide usually provides on the way about the history of the town, buildings visible from the road, and the like. Note that the language of the guidance is not limited to Japanese, and can be set to any language.

Note that, the action control unit 250 may change the expression of the avatar or change the movement of the avatar according to the content of the guidance information for the user. For example, in a case in which the guided spot and/or event is a fun spot and/or event, the expression of the avatar may be changed to a pleasant expression, or the movement of the avatar may be changed to a lively dance. Furthermore, the action control unit 250 may transform the avatar in accordance with the content of the spot and/or the event. For example, in a case in which the spot to which the user is guided relates to a historical figure, the action control unit 250 may transform the avatar into an avatar that looks like the person.

Furthermore, the action control unit 250 may generate an image of the avatar to cause the avatar to have a tablet terminal drawn in a virtual space and perform an operation of drawing information of the spot and/or the event on the tablet terminal. In this case, by transmitting the information displayed on the tablet terminal to the mobile terminal device of the user 10, it is possible to express as if the avatar is performing an operation in which the information of the spot and/or the event is transmitted from the tablet terminal to the mobile terminal device of the user 10 by e-mail, the information of the spot and/or the event is transmitted on a message application, or the like. Furthermore, in this case, the user 10 can view the spot and/or the event displayed on the user's mobile terminal device.

### Thirty-First Embodiment

For example, even when not talking with the user, the robot 100 investigates information regarding a person the user is worried about and provides advice.

An action system of the robot 100 includes an emotion determination unit 232 that determines an emotion of users 10, 11, and 12 or an emotion of the robot 100; and an action determination unit 236 that generates an action content of the robot 100 for an action of the user and the emotion of the users 10, 11, and 12 or the emotion of the robot 100 based on an interaction function of causing the users 10, 11, and 12 and the robot 100 to interact with each other, and determines an action of the robot 100 corresponding to the action content. In a case in which that the users 10, 11, and 12 are determined to be specific users including an individual living alone in solitude, the action determination unit 236 switches the mode to a specific mode in which an action of the robot is determined at a higher communication frequency than a communication frequency in a normal mode in which an action of the robot is determined for the users 10, 11, and 12, other than the specific user.

The action determination unit 236 can set the specific mode, separately from the normal mode, and cause the specific mode to function as support for an elderly person living alone. In other words, in a case in which the robot 100 detects a situation of the user and determines that the user is a person living alone since the spouse has passed away or the child has become independent and left home, the action determination unit 236 makes gestures and utterances to the user more actively than in the normal mode, and increases the frequency of communication between the user and the robot 100 (switching to the specific mode).

The communication includes, in addition to interactions, a special response to a specific user, for example, a confirmation action in which the robot 100 intentionally makes a change in the life (for example, turning off the light, sounding an alarm, or the like) and confirms a response action to the change in the life, and the confirmation action is also subject to counting. The confirmation action can be referred to as an indirect communication action.

In addition, if there is no conversation with the robot 100 for a certain period of time, a preset emergency contact is reached.

According to the function of supporting the elderly living alone, the robot can serve as a conversation partner for elderly people living alone whose spouses have passed away earlier or whose children have left home to be independent. It's also good for keeping their brains active. In addition, if there is no conversation with the robot 100 for a certain period of time, a preset emergency contact can also be reached.

Note that, not only for the elderly, but also for individuals living alone in solitude, it is effective to set the people as a user target (specific user) of the function of supporting the elderly living alone.

In a case in which the action determination unit 236 determines to make an utterance as an action of the avatar, it is preferable to cause the action control unit 250 to control the avatar to utter in a changed voice in accordance with the attributes of the user (child, adult, doctor, teacher, physician, student, minor, company director, or the like).

Here, a feature of the embodiment is that the action that can be executed by the robot 100 described in the above-described example is reflected in the action of the avatar displayed in the image display area of the headset-type terminal 820. Hereinafter, when simply referred to as an “avatar”, it is assumed to indicate an avatar that is controlled by the action control unit 250 and displayed in the image display area of the headset-type terminal 820.

That is, when the control unit 228B illustrated in FIG. 15 determines an action of the avatar and displays the avatar to be presented to the user on the headset-type terminal 820, the action determination unit 236 can set the specific mode separately from the normal mode and cause the specific mode to function as support for the elderly living alone. In other words, in a case in which the action determination unit 236 causes the avatar to detect a situation of the user and determines that the user is a person living alone since the spouse has passed away or the child has become independent and left home, the avatar makes gestures and utterances to the user more actively than in the normal mode, and increases the frequency of communication between the user and the avatar (switching to the specific mode).

The communication includes, in addition to interactions, a special response to a specific user, for example, a confirmation action in which the avatar intentionally makes a change in the life (for example, turning off the light, sounding an alarm, or the like) and confirms a response action to the change in the life, and the confirmation action is also subject to counting. The confirmation action can be referred to as an indirect communication action.

In addition, if there is no conversation with the avatar for a certain period of time, a preset emergency contact is reached.

According to the function of supporting the elderly living alone, the robot can serve as a conversation partner for elderly people living alone whose spouses have passed away earlier or whose children have left home to be independent. It's also good for keeping their brains active. If there is no conversation with the avatar for a certain period of time, a preset emergency contact can also be reached.

Note that, not only for the elderly, but also for individuals living alone in solitude, it is effective to set the people as a user target (specific user) of the function of supporting the elderly living alone.

### Thirty-Second Embodiment

An action system of the robot 100 according to this embodiment includes an emotion determination unit that determines an emotion of a user or an emotion of the robot 100; and an action determination unit that generates an action content of the robot 100 with respect to an action of the user, the emotion of the user, or the emotion of the robot 100 based on an interaction function that causes the user and the robot 100 to interact with each other, and determines an action of the robot 100 corresponding to the action content, in which the emotion determination unit determines an emotion of a dependent-side user classified as a dependent based on recitation information including at least audio information of a book that a guardian-side user classified as a guardian reads aloud to the dependent-side user, and the action determination unit determines a reaction at the time of reading from an emotion of the dependent-side user, presents a book similar to the book read by the guardian-side user when the reaction of the dependent-side user is good, and presents, to the guardian-side user, information regarding a book of a different genre from the book read by the guardian-side user when the reaction of the dependent-side user is bad.

As illustrated in FIG. 2, the action determination unit 236 sets, as an interaction mode of the robot 100, a customer service interaction mode in which the robot 100 can be designated as an interaction partner when the user does not need to talk to a specific person but wants someone to listen to the user's talk, and in the customer service interaction mode, a predetermined keyword related to the specific person is excluded in the interaction with the user, and the utterance content is output.

In a case in which the user 10 wants to talk with someone even though the user is not so much in the mood to talk with a family member, a friend, a lover, or the like, the robot 100 detects the user 10 and performs customer service interactions in the style of, for example, a bartender. An NG keyword such as a family member, a friend, or a lover is set, and an utterance content that enables the NG keyword to be never output is output. In this way, the conversation content that the user 10 feels is delicate is never uttered, and the user can enjoy a gentle conversation.

In other words, even though it is not something to talk about with a family member, a friend, a lover, or the like, the robot 100 listens to what the user wants to talk about to someone. It is possible to create a customer service environment such as a bar based on a concept of providing a one-on-one customer service (more precisely, robot-to-human).

In the customer service environment, the robot 100 can contribute to stress release and the like based on the user 10's problem solution by reading the feeling from the content of conversation and proposing a recommended drink, in addition to interactions.

As described above, according to the example of the embodiment, when any intention of the user 10 (including a key operation command, an operation command, a voice command from the user 10, and automatic determination by the robot 100) is detected, the customer interaction mode is selected, and the robot 100 configures an environment in which a so-called bartender at a bar counter serves as an interaction partner (customer service environment).

Note that, in the customer service environment in the customer service interaction mode, the robot 100 may set an indoor atmosphere (lighting, music, sound effect, etc.). The atmosphere may be determined from emotion information based on an interaction with the user 10. For example, examples of lighting include relatively dark lighting and lighting using a mirror ball, examples of music include jazz and Enka, and examples of the sound effect include a sound of glass hit by something, a sound of a door opening/closing, a sound of shaking when making cocktail. However, lighting, music, and sound effect are not limited thereto, and preferably set for each situation illustrated in FIGS. 5 and 6 (emotion maps) to be described later. Furthermore, the robot 100 may store a component to be a base of odor and output the odor in accordance with speech of the user 10. Examples of the odor include a perfume odor, a burnt cheese odor of pizza, a sweet odor of crepe, a burnt soy sauce odor of baked chicken, and the like.

In addition, the action determination unit 236 sets, as an interaction mode of the avatar displayed in the image display area of the headset-type terminal 820 worn by the user 10, a customer service interaction mode in which someone can be designated as an interaction partner when the user does not need to talk to a specific person but wants someone to listen to the user's talk, and in the customer service interaction mode, a predetermined keyword related to the specific person is excluded in the interaction with the user, and the utterance content is output.

In a case in which the user 10 wants to talk with someone even though the user is not so much in the mood to talk with a family member, a friend, a lover, or the like, the avatar detects the user 10 and performs customer service interactions in the style of, for example, a bartender. An NG keyword such as a family member, a friend, or a lover is set, and an utterance content that enables the NG keyword to be never output is output. In this way, the conversation content that the user 10 feels delicate is never uttered, and the user 10 can enjoy a gentle conversation.

In other words, the avatar listens to what the user wants to talk about to someone even though that is not suitable for a family member, a friend, a lover, or the like to talk with. It is possible to create a customer service environment such as a bar based on a concept of providing a one-on-one customer service (more precisely, human-to-avatar).

In the customer service environment, the avatar can contribute to stress release or the like based on the user 10's problem resolution by reading a feeling from the content of conversation and proposing a recommended drink, in addition to the interaction.

As described above, according to the embodiment, when any intention of the user 10 (including a key operation command, an operation command, a voice command from the user 10, and automatic determination by the avatar) is detected, the customer interaction mode is selected, and the avatar configures an environment in which the avatar serves as an interaction partner like a so-called bartender at a bar counter (customer service environment).

Note that, in the customer service environment in the customer service interaction mode, the avatar (that is, the action determination unit 236) may set an indoor atmosphere (lighting, music, sound effect, etc.). The atmosphere may be determined from emotion information based on an interaction with the user 10. For example, examples of lighting include relatively dark lighting and lighting using a mirror ball, examples of music include jazz and Enka, and examples of the sound effect include a sound of glass hit by something, a sound of a door opening/closing, a sound of shaking when making cocktail. However, lighting, music, and sound effect are not limited thereto, and preferably set for each situation illustrated in FIGS. 5 and 6 (emotion maps) to be described later. Furthermore, the headset-type terminal 820 may store a component to be a base of the odor and output the odor in accordance with the speech of the user 10. Examples of the odor include a perfume odor, a burnt cheese odor of pizza, a sweet odor of crepe, a burnt soy sauce odor of baked chicken, and the like.

### Thirty-Third Embodiment

In the embodiment, the action determination unit 236 may generate an action content of the robot with respect to an action of the user, an emotion of the user, or an emotion of the robot based on an interaction function of causing the user to interact with the robot, and determine an action of the robot corresponding to the action content. At this time, the robot is set for customs, and the action determination unit 236 acquires an image of a person by an image sensor and a result of odor detection by an odor sensor, and in a case in which a preset abnormal action, abnormal expression, or abnormal odor is detected, the action determination unit determines, as an action of the robot, to notify the customs inspector of the detection.

Specifically, the robot 100 is installed at customs and detects customers passing therethrough. In addition, the robot 100 stores drug odor data and explosives odor data, and also stores data regarding behavior, facial expressions, suspicious behavior, and the like of criminals. When a customer passes through, the action determination unit 236 acquires an image of the customer by the image sensor and a result of odor detection by the odor sensor, and in a case in which suspicious behavior, suspicious facial expression, drug odor, or explosives odor is detected, the action determination unit determines, as an action of the robot 100, to notify the customs inspector of the detection.

As in the first embodiment, the action determination unit 236 of the control unit 228B acquires an image of a person by the image sensor or a result of odor detection by the odor sensor, and in a case in which a preset abnormal action, abnormal facial expression, or abnormal odor is detected, the action determination unit determines, as an action of the avatar, to notify the customs inspector of the detection.

Specifically, the image sensor and the odor sensor are installed at customs and detect customers passing therethrough. In addition, the agent system 800 stores drug odor data and explosives odor data, and also stores data regarding behavior, facial expressions, suspicious behavior, and the like of criminals. When a customer passes through, the action determination unit 236 acquires an image of the customer by the image sensor and a result of odor detection by the odor sensor, and in a case in which suspicious behavior, suspicious facial expression, drug odor, or explosives odor is detected, the action determination unit determines, as an action of the avatar, to notify the customs inspector of the detection.

In particular, in a case in which the action control unit 250 detects a preset abnormal action, abnormal facial expression, or abnormal odor, the action control unit transmits a notification message to the customs inspector while causing the avatar to perform an operation of notifying the customs inspector of the detection, and causes the avatar to state that the abnormal action, abnormal facial expression, or abnormal odor has been detected. At this time, it is preferable to operate the avatar with a look corresponding to the detected content. For example, in a case in which drug odor is detected, the avatar is operated by switching the outfit of the avatar to an outfit that looks like a handler of a drug-sniffing dog. In a case in which explosives odor is detected, the avatar is operated by switching the outfit of the avatar to an outfit that looks like an explosives disposal team.

Although the disclosure has been described with reference to the embodiments above, the technical scope of the disclosure is not limited to the scope described in the embodiments. It is apparent to those skilled in the art that various modifications or improvements can be made to the above embodiments. It is apparent from the description of the claims that a mode to which such modifications or improvements are added can also be included in the technical scope of the disclosure.

It should be noted that the order of execution of each processing such as operations, procedures, steps, and stages in the devices, systems, programs, and methods shown in the claims, the specification, and the drawings can be realized in any order unless “before”, “prior to”, or the like is explicitly stated, and unless the output of the previous processing is used in the later processing. Even if the operation flow in the claims, the specification, and the drawings is described using “first,”, “next,”, and the like for convenience, it does not mean that it is essential to perform in this order.

1. A wearable biometric monitoring apparatus comprising:
a housing configured to be worn on a body portion of a user, the housing including:
a strap assembly configured to secure the housing to the body portion, and
an internal cavity configured to receive electronic components;
a sensor assembly mounted within the housing, the sensor assembly including:
a photoplethysmography sensor including a light-emitting element configured to emit light toward skin tissue and a photodetector configured to detect reflected light and generate a heart rate signal,
a three-axis accelerometer configured to generate motion signal data representing acceleration along three orthogonal axes, and
a skin temperature sensor configured to generate temperature signal data;
a wireless transmitter configured to transmit a combined data stream to a processing device;
processing circuitry coupled to the sensor assembly, the processing circuitry configured to:
sample the heart rate signal at a first sampling rate to generate heart rate sample data,
sample the motion signal data at a second sampling rate to generate motion sample data,
sample the temperature signal data at a third sampling rate to generate temperature sample data,
multiplex the heart rate sample data, the motion sample data, and the temperature sample data into the combined data stream, and
control the wireless transmitter to transmit the combined data stream; and
a display configured to render an avatar, wherein the avatar provides health advice based on analysis of the combined data stream.

2. The apparatus of claim 1, wherein the processing circuitry is further configured to:
estimate stress levels of the user based on at least one of the heart rate signal or the temperature signal data, and
generate the health advice based on the estimated stress levels.

3. The apparatus of claim 1, wherein the processing circuitry is further configured to:
estimate fatigue levels of the user based on the motion signal data and the heart rate signal, and
generate health advice regarding rest or relaxation based on the estimated fatigue levels.

4. The apparatus of claim 1, wherein the avatar is configured to provide health advice comprising at least one of:
exercise recommendations,
dietary guidance, or
stress relief methods.

5. The apparatus of claim 1, wherein the processing circuitry is further configured to:
detect lifestyle habits of the user based on patterns in the motion signal data over time, and
store the detected lifestyle habits in history data.

6. The apparatus of claim 1, wherein the processing circuitry is further configured to:
estimate health states of the user based on the combined data stream, and
recognize the estimated health states as mental and physical states of the user.

7. The apparatus of claim 6, wherein the processing circuitry is further configured to:
in a case in which the health states indicate poor health, generate advice information regarding improvement of the health states.

8. The apparatus of claim 1, wherein the processing circuitry is further configured to:
detect an increase in the heart rate signal, and
in response to the detected increase, estimate emotion values for anxiety or fear to be high.

9. The apparatus of claim 1, wherein the processing circuitry is further configured to:
detect that the temperature signal data indicates a body temperature exceeding an average body temperature, and
in response to the detected temperature, estimate an emotion value for suffering or hardship to be high.

10. The apparatus of claim 1, wherein the processing circuitry is further configured to:
detect, from the motion signal data, that the user is performing physical activity, and
in response to the detected physical activity, estimate an emotion value for pleasant to be high.

11. The apparatus of claim 1, wherein the avatar is further configured to support diet by transforming into an appearance of a virtual user having an ideal physique.

12. The apparatus of claim 1, wherein the processing circuitry is further configured to:
determine an emotion value indicating an emotion of the user based on the sensor assembly data, and
determine an emotion value of the avatar based on the emotion of the user.

13. The apparatus of claim 1, wherein the processing circuitry is further configured to:
store event data in history data, the event data including:
an emotion value determined based on the sensor assembly data, and
data including an action of the user.

14. The apparatus of claim 1, wherein the processing circuitry is further configured to:
estimate a mental state of the user based on the combined data stream, and
generate health advice based on the estimated mental state.

15. The apparatus of claim 1, wherein the display comprises a display of smart glasses configured to be worn by the user.

16. The apparatus of claim 1, wherein the processing circuitry is further configured to:
periodically detect data related to exercise, diet, and health of the user, and
store the detected data in history data.

17. The apparatus of claim 1, wherein the housing further comprises a speaker configured to output a voice having directivity toward ears of the user.

18. A wearable biometric monitoring apparatus comprising:
a housing configured to be worn on a wrist of a user, the housing including:
a strap assembly configured to secure the housing around the wrist,
an internal cavity configured to receive electronic components, and
a speaker capable of outputting a voice having directivity toward ears of the user;
a sensor assembly mounted within the housing, the sensor assembly including:
a photoplethysmography sensor configured to detect a heart rate of the user,
a three-axis accelerometer configured to detect motion and orientation of the user, and
a temperature sensor configured to detect a body temperature of the user;
a wireless communication interface configured to communicate with a remote server;
processing circuitry configured to:
detect states of the user from the sensor assembly,
estimate stress levels and fatigue levels of the user based on the detected states,
estimate health states and lifestyle habits of the user based on the detected states,
determine emotion values based on the heart rate, body temperature, and motion data,
store event data including the emotion values and user actions in history data, and
generate health advice based on the estimated health states and lifestyle habits; and
a display configured to render an avatar that provides the health advice to the user, wherein in a case in which the processing circuitry determines to support diet as an action of the avatar, the avatar transforms into an appearance of a virtual user having an ideal physique.

19. The apparatus of claim 18, wherein the processing circuitry is further configured to:
in a case in which the stress levels are estimated to be high, cause the speaker to output advice information including a method of relieving stress or a relaxation method.

20. A method for providing health advice to a user via a wearable apparatus, the method comprising:
sensing, by a photoplethysmography sensor mounted in a housing worn on a body portion of the user, a heart rate signal of the user;
sensing, by a three-axis accelerometer mounted in the housing, motion signal data representing acceleration of the user;
sensing, by a temperature sensor mounted in the housing, temperature signal data representing a body temperature of the user;
estimating, by processing circuitry, at least one of stress levels, fatigue levels, health states, or lifestyle habits of the user based on the heart rate signal, the motion signal data, and the temperature signal data;
determining, by the processing circuitry, emotion values of the user based on the sensed signals;
storing, in a storage medium, event data including the emotion values and actions of the user;
generating, by the processing circuitry, health advice based on the estimated health states and lifestyle habits; and
rendering, on a display of the wearable apparatus, an avatar that provides the generated health advice to the user.
